(19) United States
(12) Patent Application Publication
(10) Pub. No.: US 2023/0031853 A1
(43) Pub. Date: Feb. 2, 2023

(54) COMPOSITIONS AND METHODS FOR THE CYTOPLASMIC DELIVERY OF ANTIBODIES AND OTHER PROTEINS

(71) Applicant: THE TRUSTEES OF THE UNIVERSITY OF PENNSYLVANIA, Philadelphia, PA (US)

(72) Inventors: Andrew TSOURKAS, Bryn Mawr, PA (US); Hejia Henry WANG, Philadelphia, PA (US)

(73) Assignee: THE TRUSTEES OF THE UNIVERSITY OF PENNSYLVANIA, Philadelphia, PA (US)

(21) Appl. No.: 17/294,401

(22) PCT Filed: Nov. 14, 2019
(86) PCT No.: PCT/US2019/061575

§ 371 Date: May 15, 2021

Related U.S. Application Data
(60) Provisional application No. 62/768,034, filed on Nov. 15, 2018.

(51) Int. Cl. A61K 47/68; A61K 9/51; C07K 16/40; C07K 16/18; A61P 35/00; C07K 16/32; A61K 47/64; A61K 47/65

(52) U.S. Cl. CPC A61K 47/6807; A61K 9/5123; C07K 16/40; C07K 16/18; A61K 47/6849; A61P 35/00; C07K 16/32; A61K 47/6455; A61K 47/65; C07K 2317/31; C07K 2319/30; C07K 2317/622; C07K 2317/569; C07K 2317/76; C07K 2317/77; C07K 2319/33; A61K 2039/505

(57) ABSTRACT

The invention provides compositions and methods for the cytoplasmic delivery of antibodies and other proteins. Specifically, provided herein are compositions having an anionic polypeptide and a cationic transfection agent for facilitating the cytoplasmic delivery of an antibody or a protein.

### GOVERNMENT INTEREST STATEMENT

This invention was supported by Grant Number F30 CA221385-01 awarded by the National Institutes of Health (NIH). The United States government has certain rights in the invention.

### FIELD OF THE INVENTION

The invention relates to compositions and methods for the cytoplasmic delivery of antibodies and other proteins. Specifically, provided herein are compositions having an anionic polypeptide and a cationic transfection agent for facilitating the cytoplasmic delivery of an antibody or a protein.

### BACKGROUND OF THE INVENTION

Many intracellular therapeutic targets are not susceptible to small molecule drugs, because they lack natural ligands or even ligand binding sites. Currently, most approved small-molecule therapeutics target proteins such as enzymes and receptors that contain small, but critical, binding pockets amenable to modulation by small molecule drugs, but many potential therapeutic targets lack such pockets.

Moreover, even if a small molecule drug can bind a desired target, it may not effectively inhibit protein function. Small molecule drugs capable of disrupting interactions between two proteins have been particularly difficult to identify. Furthermore, even where a small molecule drug is identified, it must be able to reach its target site with good pharmacokinetic properties and minimal off-target toxicity. These stringent requirements have led to long development times and only a very small fraction of small molecule drugs have been successfully translated into the clinic, despite decades of research and countless high-throughput screens.

Therapeutic monoclonal antibodies have had considerable success as cancer therapeutics, but their inability to cross cell membranes has restricted their targets to secreted or membrane-associated antigens.

Currently, there are two broad approaches for cytoplasmic antibody delivery: 1) cell-penetrating peptide (CPP) based and 2) protein transfection-based methods. CPPs are short, poly-cationic peptides that can induce endocytic cellular uptake of not only themselves, but also cargo conjugated to them. Although CPPs are highly effective at delivering large cargos into the endosome-lysosome system, a vanishingly small fraction of cargos actually escape into the cytosol, where most therapeutically relevant targets are. Because of this endosome escape problem, CPPs have been limited to delivering enzymes and other proteins capable of greatly amplifying their effects. Since it is likely that stoichiometric amounts of inhibitory antibodies need to be delivered relative to their targets for a sustained biological effect, tremendous advances in CPP-mediated endosome escape must be made before they become viable for cytoplasmic antibody delivery.

Like nucleic acid transfection, in protein transfection, cargo proteins are encapsulated into lipid or polymer nanoparticles that can induce endocytic uptake and then destabilize the endosome membrane to allow for cytoplasmic release of cargo proteins. This takes advantage of advances in nucleic acid delivery that have led to lipid and polymer formulations much better at endosomal escape than CPPs. Although many protein transfection systems have been developed, they should be evaluated cautiously—most were developed using fluorescently labeled model proteins, which does not allow one to easily distinguish between proteins bound to the cell surface, proteins stuck in endosomes, and proteins delivered to the cytoplasm, dramatically increasing the potential for false positives. In fact, evaluation of 4 commercially available protein transfection systems for antibody delivery using a very stringent Cre-recombinase based cytoplasmic delivery reporter system revealed that none were able to deliver to >6% of cells, indicating that these systems suffer from poor efficacy, poor reproducibility, or likely both. Finally, none of the systems described thus far have managed to deliver antibodies cytoplasmically in vivo, likely due to poor serum stability. However, recent progress in delivering Cas9 proteins, which are as large as antibodies, with lipid nanoparticles for genome editing in vivo indicates that protein transfection is a viable strategy for cytoplasmic antibody delivery. Making therapeutic cytoplasmically delivered antibodies a reality, though, requires 1) a reproducible method to efficiently encapsulate a large variety of antibodies into stable lipid or polymer nanoparticle formulations, 2) stringent testing of formulations using a cell reporter assay that only detects cytoplasmic protein delivery, and 3) demonstration that cytoplasmically delivered antibodies can inhibit therapeutically relevant targets in both cell culture and model organisms.

If antibodies could efficiently be delivered into the cytosol of living cells, it would significantly increase the number of possible druggable targets. Antibodies can be developed to bind nearly any exposed protein epitope, with high specificity and affinity. There are a countless number of therapeutic possibilities that could be pursued if antibodies could effectively be delivered into cells, from inhibiting protein function, to driving proteins interactions, to targeting intracellular proteins for degradation. Not surprisingly, numerous attempts have been made to deliver antibodies into cells, but a robust and efficient approach has yet to be identified.

Accordingly, there exists a need to develop a modular approach to efficiently deliver antibodies and other proteins into the cytoplasm of living cells.

### SUMMARY OF THE INVENTION

In one aspect, provided herein are compositions comprising: an antibody or other protein; an anionic polypeptide, an anionic polymer or an anionic nucleic acid; and a cationic transfection agent, wherein the presence of said anionic polypeptide, anionic polymer or anionic nucleic acid and said cationic transfection agent in said composition facilitate cytoplasmic delivery of said antibody or other protein.

In one aspect, provided herein are compositions comprising: an antibody or other protein; an anionic polypeptide; and a cationic transfection agent, wherein the anionic polypeptide comprises a plurality of negatively charged amino acid residues, and wherein the presence of said anionic polypeptide and said cationic transfection agent in said composition facilitate cytoplasmic delivery of said antibody or other protein. In one embodiment, said antibody is operably linked to an antibody binding domain (AbBD). In some embodiments, said antibody binding domain is operably linked to a photoreactive amino acid group, for example, benzoylphenylalanine (BPA) resulting in a photoreactive antibody binding domain (pAbBD). In one example, at least 20% of residues in said anionic polypeptide are negatively charged amino acid residues (e.g., aspartic acid residues, glutamic acid residues, unnatural amino acids, or combinations thereof). In another example, the cationic transfection agent is an ionizable lipid, lipid-like, and/or polymeric particle. In another example, the particle is a nanoparticle.

In another aspect, provided herein are compositions comprising: an antibody or other protein; an anionic polypeptide; and an agent that induces protein degradation, wherein said anionic polypeptide comprises a plurality of negatively charged amino acid residues. In some embodiments, a composition described herein includes an agent that modifies the function of a target protein; an agent that induces nuclear, cytoplasmic, membrane, or membrane-associated proteins to be sorted into subcellular compartments; or a combination thereof.

In another aspect, provided herein are conjugates comprising an antibody binding domain (AbBD) operably linked, ligated or fused to an anionic polypeptide comprising a plurality of negatively charged amino acid residues. In some embodiments, the antibody binding domain is operably linked to a photoreactive amino acid group, for example, benzoylphenylalanine (BPA) resulting in a photoreactive antibody binding domain (pAbBD). In one example, at least 20% of residues in said anionic polypeptide are negatively charged amino acid residues (e.g., aspartic acid residues, glutamic acid residues, unnatural amino acids, or combinations thereof).

In one aspect, provided herein are conjugates comprising: a protein operably linked, ligated, conjugated or fused to an anionic nucleic acid. In some embodiments, the protein is a single chain protein. In certain embodiments, the protein is operably linked to the anionic nucleic acid. In some embodiments, the single chain protein is a single chain protein, as described herein. In various embodiments, the anionic nucleic acid is operably linked, ligated, conjugated or fused to an antibody. In particular embodiments, the anionic nucleic acid is operably linked, ligated, conjugated or fused to an AbBD.

In another aspect, provided herein are methods of delivering an antibody or other protein to cell cytoplasm in a subject, comprising: providing a composition described herein; and administering said composition to said subject.

In one aspect, the invention provides a method of delivering an method of delivering an antibody or other protein to cytoplasm of a cell in a subject, comprising: providing a composition described herein; and administering said composition to said subject, wherein the composition comprises: the antibody or the other protein; an anionic nucleic acid; and a cationic transfection agent. In some embodiments, the protein is a single chain protein. In some embodiments, the nucleic acid comprises a plurality of negatively charged residues, i.e., is an anionic nucleic acid. In various embodiments, the anionic nucleic acid is operably linked, ligated, conjugated or fused to an antibody. In particular embodiments, the anionic nucleic acid is operably linked, ligated, conjugated or fused to an AbBD.

In another aspect, the invention provides a method of treating a disease or disorder in a subject, comprising: delivering a composition described herein to cell cytoplasm in the subject.

In another aspect, the invention provides a method of manufacturing a composition for a cytoplasmic delivery, comprising: covalently linking, ligating, or fusing an antibody or other protein with an anionic polypeptide in order to prepare a conjugate; and mixing or complexing a cationic transfection agent with said conjugate.

In one aspect, the invention provides a method of manufacturing a composition for a cytoplasmic delivery, comprising: covalently linking, ligating, or fusing a protein to an nucleic acid in order to prepare a conjugate; and mixing or complexing a cationic transfection agent with said conjugate. In some embodiments, the nucleic acid comprises a plurality of negatively charged residues, i.e., is an anionic nucleic acid. In an embodiment, the protein is a single chain protein. In some embodiments, the single chain protein is a single chain protein, as described herein. In various embodiments, the anionic nucleic acid is operably linked, ligated, conjugated or fused to an antibody. In particular embodiments, the anionic nucleic acid is operably linked, ligated, conjugated or fused to an AbBD.

In another aspect, the antibody or protein is further labeled with an imaging agent, drug, and/or toxin.

In one aspect, the invention provides compositions comprising: a protein; an nucleic acid; and a cationic transfection agent, wherein the nucleic acid comprises a plurality of negatively charged residues, and wherein the presence of the anionic nucleic acid and the cationic transfection agent in the composition facilitate cytoplasmic delivery of said antibody or other protein. In another aspect, the protein is single chain protein. In an embodiment, the single chain protein is further labeled with an imaging agent, drug, and/or toxin. In various embodiments, the anionic nucleic acid is operably linked, ligated, conjugated or fused to an antibody. In particular embodiments, the anionic nucleic acid is operably linked, ligated, conjugated or fused to an AbBD.

In one aspect, provided herein are methods for sensitizing a tumor cell to a chemotherapeutic agent, the method comprising administering to cytoplasm of the tumor cell: (i) a conjugate comprising an antibody binding domain (AbBD) operably linked, ligated or fused to an anionic polypeptide comprising a plurality of negatively charged amino acid residues or (ii) a cell recombinantly expressing the conjugate of (i).

In another aspect, provided herein are methods for sensitizing a tumor cell to a chemotherapeutic agent, the method comprising administering to cytoplasm of the tumor cell: (i) a conjugate comprising a protein operably linked, ligated, conjugated or fused to a nucleic acid, wherein the nucleic acid comprises a plurality of negatively charged residues, i.e., is an anionic nucleic acid, and wherein presence of the anionic nucleic acid and the cationic transfection agent in the composition facilitate cytoplasmic delivery of said antibody or other protein, or (ii) a cell recombinantly expressing the conjugate of (i). In an embodiment, the protein is single chain protein. In an embodiment, the anionic nucleic acid is operably linked, ligated, conjugated or fused to an antibody. In particular embodiments, the anionic nucleic acid is operably linked, ligated, conjugated or fused to an AbBD.

In one aspect, provided herein are methods for decreasing or inhibiting growth of a tumor cell, the method comprising administering to cytoplasm of the tumor cell in a subject in need thereof a composition comprising an antibody or other protein; an anionic polypeptide; and a cationic transfection agent, wherein said anionic polypeptide comprises a plurality of negatively charged amino acid residues, and wherein the presence of said anionic polypeptide and said cationic transfection agent in said composition facilitate cytoplasmic delivery of said antibody or other protein.

In another aspect, provided herein are methods for decreasing or inhibiting growth of a tumor cell, the method comprising administering to cytoplasm of the tumor cell in a subject in need thereof a composition comprising: a protein; an anionic nucleic acid; and a cationic transfection agent, wherein presence of the anionic nucleic acid and the cationic transfection agent in the composition facilitate cytoplasmic delivery of the protein. In an embodiment, the protein is a single chain protein. In an embodiment, the protein is operably linked to the anionic nucleic acid. In various embodiments, the anionic nucleic acid is operably linked, ligated, conjugated or fused to an antibody. In particular embodiments, the anionic nucleic acid is operably linked, ligated, conjugated or fused to an AbBD.

In one aspect, provided herein are methods for inhibiting NF-kB transcription and/or reducing RelA nuclear translocation a cancer cell, the method comprising administering to cytoplasm of the cancer cell in a subject in need thereof a composition comprising an antibody or other protein; an anionic polypeptide; and a cationic transfection agent, wherein said anionic polypeptide comprises a plurality of negatively charged amino acid residues, and wherein the presence of said anionic polypeptide and said cationic transfection agent in said composition facilitate cytoplasmic delivery of said antibody or other protein.

In another aspect, provided herein are methods for inhibiting NF-kB transcription and/or reducing RelA nuclear translocation a cancer cell, the method comprising administering to cytoplasm of the cancer cell in a subject in need thereof a composition comprising a protein; an anionic nucleic acid; and a cationic transfection agent, wherein presence of the anionic nucleic acid and the cationic transfection agent in the composition facilitate cytoplasmic delivery of the single chain protein. In an embodiment, the protein is a single chain protein. In an embodiment, the protein is operably linked to the anionic nucleic acid. In some embodiments, the anionic nucleic acid is operably linked, ligated, conjugated or fused to an antibody. In particular embodiments, the anionic nucleic acid is operably linked, ligated, conjugated or fused to an AbBD.

Other features and advantages of this invention will become apparent from the following detailed description, examples, and figures. It should be understood, however, that the detailed description and the specific examples while indicating preferred embodiments are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of this disclosure, the inventions of which can be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIGS. 1A-1B respectively show a schematic depicting light activated site-specific conjugation of IgG with pAbBD and reducing SDS-PAGE gels of various human IgG subclasses alone or after photocrosslinking with a pAbBD. FIG. 1A shows irradiation with non-damaging long-wavelength UV light allows for covalent attachment of pAbBD with attached cargo (green star). The photoreactive amino acid (e.g., BPA) is represented by a yellow circle. In FIG. 1B the reducing SDS-PAGE gels of various human IgG subclasses alone or after photocrosslinking with a pAbBD, nearly 100% crosslinking is achieved.

FIGS. 2A-2C respectively show a schematic of proximity-based sortase ligation (PBSL), capture of the expressed recombinant protein and the efficiency of PBSL. FIG. 2A shows two binding partners are used to bring the sortase recognition motif (LPXTG) into close proximity with sortase, to increase the ligation efficiency with a peptide that possesses an N-terminal glycine. The peptide can be labeled with any chemical moiety, e.g., imaging agent, drug, hapten, etc. (red star). FIG. 2B shows that when SpyCatcher and SpyTag are employed as binding domains, ˜80% of the expressed recombinant protein can be captured. FIG. 2C shows the efficiency of ligation is >95% in the PBSL system and is completed in 4-6 hours.

FIGS. 3A-3B respectively show formation of IgG-ApP cationic lipid complexes and cytoplasmic delivery of the IgG-ApP cationic lipid complexes and detection by fluorescence of splitGFP complementation. FIG. 3A shows negatively charged IgG-ApP conjugates can be complexed with cationic lipids. FIG. 3B shows IgG-ApP lipid complexes are taken up into reporter cell lines expressing splitGFP(1-10) in the cytoplasm. The lipids allow escape of IgG-ApP into the cytoplasm. In the cytoplasm, splitGFP complementation occurs between splitGFP(1-10) and the splitGFP S11 peptide resulting in turn-on splitGFP fluorescence.

FIGS. 4A-4D show in vitro splitGFP complementation and fluorescence of pAbBD-S11 ((FIGS. 4A-4B) and Ritux-(pAbBD-S11)₂ (FIGS. 4C-4D). Time course of 400 pmol splitGFP(1-10) incubated with (FIG. 4A) pAbBD-S11 or (FIG. 4C) Ritux-(pAbBD-S11)₂ at 37° C. shows turn-on fluorescence that plateau within 6 hours. Fluorescence is linearly associated with the amount of (FIG. 4B) pAbBD-S11 or (FIG. 4D) Ritux-(pAbBD-S11)₂ added at all time points. As expected, Ritux-(pAbBD-S11)₂ fluorescence is approximately twice that of pAbBD-S11 since ˜2 pAbBD-S11 are crosslinked to each Rituximab molecule. Data are means±SEM of n=3 biological replicates.

FIGS. 5A-5B show splitGFP complementation by flow cytometry and fluorescence microscopy, respectively, in HEK293T splitGFP(1-10) cells after delivery of pAbBD-S11 or Ritux-(pAbBD-S11)₂ into the cytoplasm. pAbBD-S11 or Ritux-(pAbBD-S11)₂ were delivered by electroporation into the cytoplasm of HEK293T cells stably expressing splitGFP(1-10). After 6 hours, the cells were processed for (FIG. 5A) flow cytometry, which shows a 33.5-fold increase in median fluorescence with 40 μM pAbBD-S11 and 39.35-fold increase with 7.5 μM Ritux-(pAbBD-S11)₂, and (FIG. 5B) fluorescence microscopy, which shows diffuse splitGFP fluorescence. For Ritux-(pAbBD-S11)₂, there is nuclear depletion of splitGFP fluorescence because Ritux-(pAbBD-S11)₂ conjugates are too large to passively cross the nuclear pore complex.

FIG. 6 shows pAbBD-Dₓ/Eₓ-S11 SDS-PAGE. SDS-PAGE of pAbBD-S11 and pAbBD-Dₓ/Eₓ-S11 containing 10, 15, 20, 25, or 30 repeats of aspartic acid (D) or glutamic acid (E) used in delivery studies.

FIG. 7 shows pAbBD-Dₓ-S11 delivery with Lipofectamine 2000. 80,000 HEK293T splitGFP(1-10) cells were seeded onto each well of a 48 well plate in 180 μL media at 37° C. for 12-16 hours. Lipid nanoparticles were formed by incubating 2 μL Lipofectamine 2000 with the indicated protein (500 nM final concentration in well) in OptiMEM (20 μL final volume, pH 7.4) at 25° C. for 10 minutes. The lipid nanoparticles were then added to the cells and incubated for 6 hours at 37° C. before live-cell fluorescence microscopy. 50 μg/mL Hoechst 33342 was added 30 minutes prior to microscopy. Top panel is the splitGFP channel, which shows cytoplasmic delivery. Middle panel is the Hoechst channel, which shows all cell nuclei. Bottom panel is the splitGFP and Hoechst channel merged. Fluorescence microscopy shows greater cytoplasmic delivery (diffuse splitGFP fluorescence) with increasing aspartic acid (D) repeat length until a maximum at 20 repeats followed by a decrease with longer repeats.

FIG. 8 shows pAbBD-Eₓ-S11 delivery with Lipofectamine 2000. 80,000 HEK293T splitGFP(1-10) cells were seeded onto each well of a 48 well plate in 180 μL media at 37° C. for 12-16 hours. Lipid nanoparticles were formed by incubating 2 μL Lipofectamine 2000 with the indicated protein (500 nM final concentration in well) in OptiMEM (20 μl final volume, pH 7.4) at 25° C. for 10 min. The lipid nanoparticles were then added to the cells and incubated for 6 hours at 37° C. before live-cell fluorescence microscopy. 50 μg/mL Hoechst 33342 was added 30 minutes prior to microscopy. Top panel is the splitGFP channel, which shows cytoplasmic delivery. Middle panel is the Hoechst channel, which shows all cell nuclei. Bottom panel is the splitGFP and Hoechst channel merged. Fluorescence microscopy shows greater cytoplasmic delivery (diffuse splitGFP fluorescence) with increasing glutamic acid (E) repeat length.

FIG. 9 shows pAbBD-Dₓ-S11 delivery with Lipofectamine RNAiMax. 80,000 HEK293T splitGFP(1-10) cells were seeded onto each well of a 48 well plate in 180 μL media at 37° C. for 12-16 hours. Lipid nanoparticles were formed by incubating 2 μL Lipofectamine RNAiMax with the indicated protein (500 nM final concentration in well) in OptiMEM (20 μL final volume, pH 7.4) at 25° C. for 10 min. The lipid nanoparticles were then added to the cells and incubated for 6 hours at 37° C. before live-cell fluorescence microscopy. 50 μg/mL Hoechst 33342 was added 30 minutes prior to microscopy. Top panel is the splitGFP channel, which shows cytoplasmic delivery. Middle panel is the Hoechst channel, which shows all cell nuclei. Bottom panel is the splitGFP and Hoechst channel merged. Fluorescence microscopy shows greater cytoplasmic delivery (diffuse splitGFP fluorescence) with increasing aspartic acid (D) repeat length until a maximum at 25 repeats and then a small decrease at D30.

FIG. 10 shows pAbBD-D₁₀/E₁₀ delivery with Lipofectamine 2000. 80,000 HEK293T splitGFP(1-10) cells were seeded onto each well of a 48 well plate in 180 μL media at 37° C. for 12-16 hours. Lipid nanoparticles were formed by incubating the indicated amount of cationic lipid with pAbBD-D₁₀/E₁₀-S11 (500 nM final concentration in well) in OptiMEM (20 μL final volume, pH 7.4) at 25° C. for 10 min. The lipid nanoparticles were then added to the cells and incubated for 6 hours at 37° C. before determining the amount of splitGFP complementation by flow cytometry or viability by LDH assay. Negative controls undergo the same procedure, but with 500 nM pAbBD-S11 protein. The left panel shows a representative flow cytometry histogram of splitGFP fluorescence. For each cationic lipid amount used, the fold-increase in median splitGFP fluorescence as well as the percentage of splitGFP positive cells are indicated. The middle panel shows that as more cationic lipids are used during particle formation, more protein is cytoplasmically delivered as quantified by the fold-increase in median splitGFP fluorescence over the negative control. The right panel shows the relationship between the amount of cationic lipids used and viability as well as the percent of cells gated as splitGFP positive. Dashed line indicates 90% of the cell population. Data are means±SEM of n=4 biological replicates.

FIG. 11 shows pAbBD-D₁₅/E₁₅ delivery with Lipofectamine 2000. 80,000 HEK293T splitGFP(1-10) cells were seeded onto each well of a 48 well plate in 180 μL media at 37° C. for 12-16 hours. Lipid nanoparticles were formed by incubating the indicated amount of cationic lipid with pAbBD-D₁₅/E₁₅-S11 (500 nM final concentration in well) in OptiMEM (20 μL final volume, pH 7.4) at 25° C. for 10 min. The lipid nanoparticles were then added to the cells and incubated for 6 hours at 37° C. before determining the amount of splitGFP complementation by flow cytometry or viability by LDH assay. Negative controls undergo the same procedure, but with 500 nM pAbBD-S11 protein. The left panel shows a representative flow cytometry histogram of splitGFP fluorescence. For each amount of cationic lipid used, the fold-increase in median splitGFP fluorescence as well as the percentage of splitGFP positive cells are indicated. The middle panel shows that as more cationic lipids are used during particle formation, more protein is cytoplasmically delivered as quantified by the fold-increase in median splitGFP fluorescence over the negative control. The right panel shows the relationship between the amount of cationic lipids used and viability as well as the percent of cells gated as splitGFP positive. Dashed line indicates 90% of the cell population. Data are means±SEM of n=4 biological replicates.

FIG. 12 shows pAbBD-D₂₀/E₂₀ delivery with Lipofectamine 2000. 80,000 HEK293T splitGFP(1-10) cells were seeded onto each well of a 48 well plate in 180 μL media at 37° C. for 12-16 hours. Lipid nanoparticles were formed by incubating the indicated amount of cationic lipid with pAbBD-D₂₀/E₂₀-S11 (500 nM final concentration in well) in OptiMEM (20 μL final volume, pH 7.4) at 25° C. for 10 min. The lipid nanoparticles were then added to the cells and incubated for 6 hours at 37° C. before determining the amount of splitGFP complementation by flow cytometry or viability by LDH assay. Negative controls undergo the same procedure, but with 500 nM pAbBD-S11 protein. The left panel shows a representative flow cytometry histogram of splitGFP fluorescence. For each amount of cationic lipid used, the fold-increase in median splitGFP fluorescence as well as the percentage of splitGFP positive cells are indicated. The middle panel shows that as more cationic lipids are used during particle formation, more protein is cytoplasmically delivered as quantified by the fold-increase in median splitGFP fluorescence over the negative control. The right panel shows the relationship between the amount of cationic lipids used and viability as well as the percent of cells gated as splitGFP positive. Dashed line indicates 90% of the cell population. Data are means±SEM of n=4 biological replicates.

FIG. 13 shows pAbBD-D₂₅/E₂₅ delivery with Lipofectamine 2000. 80,000 HEK293T splitGFP(1-10) cells were seeded onto each well of a 48 well plate in 180 μL media at 37° C. for 12-16 hours. Lipid nanoparticles were formed by incubating the indicated amount of cationic lipid with pAbBD-D₂₅/E₂₅-S11 (500 nM final concentration in well) in OptiMEM (20 μL final volume, pH 7.4) at 25° C. for 10 min. The lipid nanoparticles were then added to the cells and incubated for 6 hours at 37° C. before determining the amount of splitGFP complementation by flow cytometry or viability by LDH assay. Negative controls undergo the same procedure, but with 500 nM pAbBD-S11 protein. The left panel shows a representative flow cytometry histogram of splitGFP fluorescence. For each amount of cationic lipid used, the fold-increase in median splitGFP fluorescence as well as the percentage of splitGFP positive cells are indicated. The middle panel shows that as more cationic lipids are used during particle formation, more protein is cytoplasmically delivered as quantified by the fold-increase in median splitGFP fluorescence over the negative control. The right panel shows the relationship between the cationic lipid amounts used and viability, as well as the percent of cells gated as splitGFP positive. Dashed line indicates 90% of the cell population. Data are means±SEM of n=4 biological replicates.

FIG. 14 shows pAbBD-D₃₀/E₃₀ delivery with Lipofectamine 2000. 80,000 HEK293T splitGFP(1-10) cells were seeded onto each well of a 48 well plate in 180 μL media at 37° C. for 12-16 hours. Lipid nanoparticles were formed by incubating the indicated amount of cationic lipid with pAbBD-D₃₀/E₃₀-S11 (500 nM final concentration in well) in OptiMEM (20 μL final volume, pH 7.4) at 25° C. for 10 min. The lipid nanoparticles were then added to the cells and incubated for 6 hours at 37° C. before determining the amount of splitGFP complementation by flow cytometry or viability by LDH assay. Negative controls undergo the same procedure, but with 500 nM pAbBD-S11 protein. The left panel shows a representative flow cytometry histogram of splitGFP fluorescence. For each cationic lipid amount used, the fold-increase in median splitGFP fluorescence, as well as the percentage of splitGFP positive cells are indicated. The middle panel shows that as more cationic lipids are used during particle formation, more protein is cytoplasmically delivered as quantified by the fold-increase in median splitGFP fluorescence over the negative control. The right panel shows the relationship between the cationic lipid amounts used and viability, as well as the percent of cells gated as splitGFP positive. Dashed line indicates 90% of the cell population. Data are means±SEM of n=4 biological replicates.

FIG. 15 shows pAbBD-Dₓ/Eₓ-S11 delivery with Lipofectamine 2000. 500 nM pAbBD-Dₓ/Eₓ-S11 with 5, 10, 15, 20, 25, or 30 repeats of aspartic acid (D) or glutamic acid (E) was delivered into HEK293T splitGFP(1-10) cells as previously described with 1 μL or 2 μL Lipofectamine 2000. Under these conditions, cell viability remained ≥90%. Flow cytometry analysis was then used to determine the fold-increase in median splitGFP fluorescence, as well as the percent of cells gated splitGFP positive. For poly-aspartic acid repeats, cytoplasmic delivery increased with repeat length until D₂₀, after which there is a significant decrease in delivery efficiency. For poly-glutamic acid repeats, cytoplasmic delivery increased with repeat length. Poly-aspartic acid and poly-glutamic acid repeats achieved similar maximal delivery efficiencies at repeat lengths of D₂₀ and E₃₀, respectively.

FIGS. 16A-16B respectively show Rituximab-(pAbBD-Dₓ/Eₓ-S11)₂ conjugate preparation with either repeats of aspartic acid or glutamic acid. Preparation of Rituximab-(pAbBD-Dₓ/Eₓ-S11)₂ conjugates in which the pAbBD has 10, 15, 20, 25, or 30 repeats of (FIG. 16A) aspartic acid (D) or (FIG. 16B) glutamic acid (E). For each protein, pAbBD was added to Ritux. at a 2:1 ratio prior to crosslinking (Pre-CL). Post-CL shows the protein following 4 hours of irradiation with 365 nm light at 4° C. Final shows the IgG-pAbBD conjugate following processing (washing with PBS to remove uncrosslinked pAbBD and concentrating) just prior to storage in −80° C.

FIG. 17 shows Ritux-(pAbBD-Dₓ/Eₓ-S11)₂ SDS-PAGE. SDS-PAGE of Ritux-(pAbBD-S11)₂ and Ritux-(pAbBD-Dₓ/Eₓ-S11)₂ containing 10, 15, 20, 25, or 30 repeats of aspartic acid (D) or glutamic acid (E) used in delivery studies.

FIG. 18 shows Rituximab-(pAbBD-Dₓ/Eₓ-S11)₂ Native Gel. Equimolar amounts of Rituximab and indicated Rituximab-(pAbBD-Dₓ/Eₓ-S11)₂ conjugates were run on a Tris-Acetate 3-8% gradient gel under non-reducing and native conditions at 150V for 2 hours. Afterwards, the gel was stained with SimplyBlue Coomassie G-250 stain. Rituximab, which has a theoretical net charge of +18 did not migrate into the gel. All Rituximab-(pAbBD-Dₓ/Eₓ-S11)₂ conjugates, however, were able to migrate down the gel. Increasing poly-aspartic acid or poly-glutamic acid repeat length increased the migration distance, suggesting a concordant decrease in the net charge of the corresponding Rituximab-(pAbBD-Dₓ/Eₓ-S11)₂ conjugate.

FIG. 19 shows Ritux-(pAbBD-Dₓ-S11)₂ delivery with Lipofectamine 2000. 80,000 HEK293T splitGFP(1-10) cells were seeded onto each well of a 48 well plate in 180 μL media at 37° C. for 12-16 hours. Lipid nanoparticles were formed by incubating 2 μL Lipofectamine 2000 with the indicated protein (500 nM final concentration in well) in OptiMEM (20 μL final volume, pH 7.4) at 25° C. for 10 min. The lipid nanoparticles were then added to the cells and incubated for 6 hours at 37° C. before live-cell fluorescence microscopy. 50 μg/mL Hoechst 33342 was added 30 minutes prior to microscopy. Top panel is the splitGFP channel, which shows cytoplasmic delivery. Middle panel is the Hoechst channel, which shows all cell nuclei. Bottom panel is the splitGFP and Hoechst channel merged. Fluorescence microscopy shows greater cytoplasmic delivery (diffuse splitGFP fluorescence with nuclear depletion; occasional puncta) with increasing aspartic acid (D) repeat length until a maximum at 25 repeats and then a small decrease at D30.

FIG. 20 shows Ritux-(pAbBD-Eₓ-S11)₂ delivery with Lipofectamine 2000. 80,000 HEK293T splitGFP(1-10) cells were seeded onto each well of a 48 well plate in 180 μL media at 37° C. for 12-16 hours. Lipid nanoparticles were formed by incubating 2 μL Lipofectamine 2000 with the indicated protein (500 nM final concentration in well) in OptiMEM (20 μL final volume, pH 7.4) at 25° C. for 10 min. The lipid nanoparticles were then added to the cells and incubated for 6 hours at 37° C. before live-cell fluorescence microscopy. 50 μg/mL Hoechst 33342 was added 30 minutes prior to microscopy. Top panel is the splitGFP channel, which shows cytoplasmic delivery. Middle panel is the Hoechst channel, which shows all cell nuclei. Bottom panel is the splitGFP and Hoechst channel merged. Fluorescence microscopy shows greater cytoplasmic delivery (diffuse splitGFP fluorescence with nuclear depletion; occasional puncta) with increasing glutamic acid (E) repeat length until a plateau beginning at 20 repeats.

FIG. 21 shows Ritux-(pAbBD-Dₓ-S11)₂ delivery with Lipofectamine RNAiMax. 80,000 HEK293T splitGFP(1-10) cells were seeded onto each well of a 48 well plate in 180 μL media at 37° C. for 12-16 hours. Lipid nanoparticles were formed by incubating 2 μL Lipofectamine RNAiMax with the indicated protein (500 nM final concentration in well) in OptiMEM (20 μL final volume, pH 7.4) at 25° C. for 10 min. The lipid nanoparticles were then added to the cells and incubated for 6 hours at 37° C. before live-cell fluorescence microscopy. 50 μg/mL Hoechst 33342 was added 30 minutes prior to microscopy. Top panel is the splitGFP channel, which shows cytoplasmic delivery. Middle panel is the Hoechst channel, which shows all cell nuclei. Bottom panel is the splitGFP and Hoechst channel merged. Fluorescence microscopy shows greater cytoplasmic delivery (diffuse splitGFP fluorescence with nuclear depletion; occasional puncta) with increasing aspartic acid (D) repeat length.

FIG. 22 shows Ritux-(pAbBD-Eₓ-S11)₂ delivery with Lipofectamine RNAiMax. 80,000 HEK293T splitGFP(1-10) cells were seeded onto each well of a 48 well plate in 180 μL media at 37° C. for 12-16 hours. Lipid nanoparticles were formed by incubating 2 μL Lipofectamine RNAiMax with the indicated protein (500 nM final concentration in well) in OptiMEM (20 μL final volume, pH 7.4) at 25° C. for 10 min. The lipid nanoparticles were then added to the cells and incubated for 6 hours at 37° C. before live-cell fluorescence microscopy. 50 μg/mL Hoechst 33342 was added 30 minutes prior to microscopy. Top panel is the splitGFP channel, which shows cytoplasmic delivery. Middle panel is the Hoechst channel, which shows all cell nuclei. Bottom panel is the splitGFP and Hoechst channel merged. Fluorescence microscopy shows greater cytoplasmic delivery (diffuse splitGFP fluorescence with nuclear depletion; occasional puncta) with increasing glutamic acid (E) repeat length.

FIG. 23 shows Ritux-(pAbBD-D₁₀/E₁₀-S11)₂ delivery with Lipofectamine 2000. 80,000 HEK293T splitGFP(1-10) cells were seeded onto each well of a 48 well plate in 180 μL media at 37° C. for 12-16 hours. Lipid nanoparticles were formed by incubating the indicated amount of cationic lipid with Ritux-(pAbBD-D₁₀/E₁₀-S11)₂ (500 nM final concentration in well) in OptiMEM (20 μL final volume, pH 7.4) at 25° C. for 10 min. The lipid nanoparticles were then added to the cells and incubated for 6 hours at 37° C. before determining the amount of splitGFP complementation by flow cytometry or viability by LDH assay. Negative controls undergo the same procedure, but with 500 nM Ritux-(pAbBD-S11)₂ protein. The left panel shows a representative flow cytometry histogram of splitGFP fluorescence. For each cationic lipid amount used, the fold-increase in median splitGFP fluorescence, as well as the percentage of splitGFP positive cells are indicated. The middle panel shows that as more cationic lipids are used during particle formation, more protein is cytoplasmically delivered as quantified by the fold-increase in median splitGFP fluorescence over the negative control. The right panel shows the relationship between the cationic lipid amounts used and viability as well as the percent of cells gated as splitGFP positive. Dashed line indicates 90% of the cell population. Data are means±SEM of n=4 biological replicates.

FIG. 24 shows Ritux-(pAbBD-D₁₅/E₁₅-S11)₂ delivery with Lipofectamine 2000. 80,000 HEK293T splitGFP(1-10) cells were seeded onto each well of a 48 well plate in 180 μL media at 37° C. for 12-16 hours. Lipid nanoparticles were formed by incubating the indicated cationic lipid amount with Ritux-(pAbBD-D₁₅/E₁₅-S11)₂ (500 nM final concentration in well) in OptiMEM (20 μL final volume, pH 7.4) at 25° C. for 10 min. The lipid nanoparticles were then added to the cells and incubated for 6 hours at 37° C. before determining the amount of splitGFP complementation by flow cytometry or viability by LDH assay. Negative controls undergo the same procedure, but with 500 nM Ritux-(pAbBD-S11)₂ protein. The left panel shows a representative flow cytometry histogram of splitGFP fluorescence. For each cationic lipid amount used, the fold-increase in median splitGFP fluorescence, as well as the percentage of splitGFP positive cells are indicated. The middle panel shows that as more cationic lipids are used during particle formation, more protein is cytoplasmically delivered as quantified by the fold-increase in median splitGFP fluorescence over the negative control. The right panel shows the relationship between the cationic lipid amounts used and viability, as well as the percent of cells gated as splitGFP positive. Dashed line indicates 90% of the cell population. Data are means±SEM of n=4 biological replicates.

FIG. 25 shows Ritux-(pAbBD-D₂₀/E₂₀-S11)₂ delivery with Lipofectamine 2000. 80,000 HEK293T splitGFP(1-10) cells were seeded onto each well of a 48 well plate in 180 μL media at 37° C. for 12-16 hours. Lipid nanoparticles were formed by incubating the indicated cationic lipid amount with Ritux-(pAbBD-D₂₀/E₂₀-S11)₂ (500 nM final concentration in well) in OptiMEM (20 μL final volume, pH 7.4) at 25° C. for 10 min. The lipid nanoparticles were then added to the cells and incubated for 6 hours at 37° C. before determining the amount of splitGFP complementation by flow cytometry or viability by LDH assay. Negative controls undergo the same procedure, but with 500 nM Ritux-(pAbBD-S11)₂ protein. The left panel shows a representative flow cytometry histogram of splitGFP fluorescence. For each cationic lipid amount used, the fold-increase in median splitGFP fluorescence as well as the percentage of splitGFP positive cells are indicated. The middle panel shows that as more cationic lipids are used during particle formation, more protein is cytoplasmically delivered as quantified by the fold-increase in median splitGFP fluorescence over the negative control. The right panel shows the relationship between the cationic lipid amounts used and viability, as well as the percent of cells gated as splitGFP positive. Dashed line indicates 90% of the cell population. Data are means±SEM of n=4 biological replicates.

FIG. 26 shows Ritux-(pAbBD-D₂₅/E₂₅-S11)₂ delivery with Lipofectamine 2000. 80,000 HEK293T splitGFP(1-10) cells were seeded onto each well of a 48 well plate in 180 μL media at 37° C. for 12-16 hours. Lipid nanoparticles were formed by incubating the indicated amount of cationic lipid with Ritux-(pAbBD-D₂₅/E₂₅-S11)₂ (500 nM final concentration in well) in OptiMEM (20 μL final volume, pH 7.4) at 25° C. for 10 min. The lipid nanoparticles were then added to the cells and incubated for 6 hours at 37° C. before determining the amount of splitGFP complementation by flow cytometry or viability by LDH assay. Negative controls undergo the same procedure, but with 500 nM Ritux-(pAbBD-S11)₂ protein. The left panel shows a representative flow cytometry histogram of splitGFP fluorescence. For each cationic lipid amount used, the fold-increase in median splitGFP fluorescence as well as the percentage of splitGFP positive cells are indicated. The middle panel shows that as more cationic lipids are used during particle formation, more protein is cytoplasmically delivered as quantified by the fold-increase in median splitGFP fluorescence over the negative control. The right panel shows the relationship between the cationic lipid amounts used and viability, as well as the percent of cells gated as splitGFP positive. Dashed line indicates 90% of the cell population. Data are means±SEM of n=4 biological replicates.

FIG. 27 shows Ritux-(pAbBD-D₃₀/E₃₀-S11)₂ delivery with Lipofectamine 2000. 80,000 HEK293T splitGFP(1-10) cells were seeded onto each well of a 48 well plate in 180 μL media at 37° C. for 12-16 hours. Lipid nanoparticles were formed by incubating the indicated amount of cationic lipid with Ritux-(pAbBD-D₃₀/E₃₀-S11)₂ (500 nM final concentration in well) in OptiMEM (20 μL final volume, pH 7.4) at 25° C. for 10 min. The lipid nanoparticles were then added to the cells and incubated for 6 hours at 37° C. before determining the amount of splitGFP complementation by flow cytometry or viability by LDH assay. Negative controls undergo the same procedure, but with 500 nM Ritux-(pAbBD-S11)₂ protein. The left panel shows a representative flow cytometry histogram of splitGFP fluorescence. For each cationic lipid amount used, the fold-increase in median splitGFP fluorescence as well as the percentage of splitGFP positive cells are indicated. The middle panel shows that as more cationic lipids are used during particle formation, more protein is cytoplasmically delivered as quantified by the fold-increase in median splitGFP fluorescence over the negative control. The right panel shows the relationship between the cationic lipid amounts used and viability as well as the percent of cells gated as splitGFP positive. Dashed line indicates 90% of the cell population. Data are means±SEM of n=4 biological replicates.

FIG. 28 shows Ritux-(pAbBD-Dₓ/Eₓ-S11)₂ delivery with Lipofectamine 2000. 500 nM Ritux-(pAbBD-Dₓ/Eₓ-S11)₂ with 5, 10, 15, 20, 25, or 30 repeats of aspartic acid (D) or glutamic acid (E) was delivered into HEK293T splitGFP(1-10) cells as previously described with 1 μL or 2 μL Lipofectamine 2000. Flow cytometry analysis was then used to determine the fold-increase in median splitGFP fluorescence, as well as the percent of cells gated splitGFP positive. For poly-aspartic acid repeats, cytoplasmic delivery increased with repeat length until D₂₅, after which there is a slight decrease at D₃₀. For poly-glutamic acid repeats, cytoplasmic delivery increased with repeat length until it hits a plateau at D₂₀. Poly-aspartic acid and poly-glutamic acid repeats achieved similar maximal delivery efficiencies at repeat lengths of D₂₅ and E₂₅, respectively.

FIG. 29 shows Ritux-(pAbBD-D₁₀/E₁₀-S11)₂ delivery with Lipofectamine RNAiMax. 80,000 HEK293T splitGFP(1-10) cells were seeded onto each well of a 48 well plate in 180 μL media at 37° C. for 12-16 hours. Lipid nanoparticles were formed by incubating the indicated amount of cationic lipid with Ritux-(pAbBD-D₁₀/E₁₀-S11)₂ (500 nM final concentration in well) in OptiMEM (20 μL final volume, pH 7.4) at 25° C. for 10 min. The lipid nanoparticles were then added to the cells and incubated for 6 hours at 37° C. before determining the amount of splitGFP complementation by flow cytometry or viability by LDH assay. Negative controls undergo the same procedure, but with 500 nM Ritux-(pAbBD-S11)₂ protein. The left panel shows a representative flow cytometry histogram of splitGFP fluorescence. For each amount of cationic lipid used, the fold-increase in median splitGFP fluorescence as well as the percentage of splitGFP positive cells are indicated. The middle panel shows that as more cationic lipids are used during particle formation, more protein is cytoplasmically delivered as quantified by the fold-increase in median splitGFP fluorescence over the negative control. The right panel shows the relationship between the amount of cationic lipids used and viability as well as the percent of cells gated as splitGFP positive. Dashed line indicates 90% of the cell population. Data are means±SEM of n=4 biological replicates.

FIG. 30 shows Ritux-(pAbBD-D₁₅/E₁₅-S11)₂ delivery with Lipofectamine RNAiMax. 80,000 HEK293T splitGFP(1-10) cells were seeded onto each well of a 48 well plate in 180 μL media at 37° C. for 12-16 hours. Lipid nanoparticles were formed by incubating the indicated amount of cationic lipid with Ritux-(pAbBD-D₁₅/E₁₅-S11)₂ (500 nM final concentration in well) in OptiMEM (20 μL final volume, pH 7.4) at 25° C. for 10 min. The lipid nanoparticles were then added to the cells and incubated for 6 hours at 37° C. before determining the amount of splitGFP complementation by flow cytometry or viability by LDH assay. Negative controls undergo the same procedure, but with 500 nM Ritux-(pAbBD-S11)₂ protein. The left panel shows a representative flow cytometry histogram of splitGFP fluorescence. For each cationic lipid amount used, the fold-increase in median splitGFP fluorescence, as well as the percentage of splitGFP positive cells are indicated. The middle panel shows that as more cationic lipids are used during particle formation, more protein is cytoplasmically delivered as quantified by the fold-increase in median splitGFP fluorescence over the negative control. The right panel shows the relationship between the cationic lipid amounts used and viability, as well as the percent of cells gated as splitGFP positive. Dashed line indicates 90% of the cell population. Data are means±SEM of n=4 biological replicates.

FIG. 31 shows Ritux-(pAbBD-D₂₀/E₂₀-S11)₂ delivery with Lipofectamine RNAiMax. 80,000 HEK293T splitGFP(1-10) cells were seeded onto each well of a 48 well plate in 180 μL media at 37° C. for 12-16 hours. Lipid nanoparticles were formed by incubating the indicated amount of cationic lipid with Ritux-(pAbBD-D₂₀/E₂₀-S11)₂ (500 nM final concentration in well) in OptiMEM (20 μL final volume, pH 7.4) at 25° C. for 10 min. The lipid nanoparticles were then added to the cells and incubated for 6 hours at 37° C. before determining the amount of splitGFP complementation by flow cytometry or viability by LDH assay. Negative controls undergo the same procedure, but with 500 nM Ritux-(pAbBD-S11)₂ protein. The left panel shows a representative flow cytometry histogram of splitGFP fluorescence. For each cationic lipid amount used, the fold-increase in median splitGFP fluorescence, as well as the percentage of splitGFP positive cells are indicated. The middle panel shows that as more cationic lipids are used during particle formation, more protein is cytoplasmically delivered as quantified by the fold-increase in median splitGFP fluorescence over the negative control. The right panel shows the relationship between the cationic lipid amounts used and viability, as well as the percent of cells gated as splitGFP positive. Dashed line indicates 90% of the cell population. Data are means±SEM of n=4 biological replicates.

FIG. 32 shows Ritux-(pAbBD-D₂₅/E₂₅-S11)₂ delivery with Lipofectamine RNAiMax. 80,000 HEK293T splitGFP(1-10) cells were seeded onto each well of a 48 well plate in 180 μL media at 37° C. for 12-16 hours. Lipid nanoparticles were formed by incubating the indicated amount of cationic lipid with Ritux-(pAbBD-D₂₅/E₂₅-S11)₂ (500 nM final concentration in well) in OptiMEM (20 μL final volume, pH 7.4) at 25° C. for 10 min. The lipid nanoparticles were then added to the cells and incubated for 6 hours at 37° C. before determining the amount of splitGFP complementation by flow cytometry or viability by LDH assay. Negative controls undergo the same procedure, but with 500 nM Ritux-(pAbBD-S11)₂ protein. The left panel shows a representative flow cytometry histogram of splitGFP fluorescence. For each cationic lipid amount used, the fold-increase in median splitGFP fluorescence, as well as the percentage of splitGFP positive cells are indicated. The middle panel shows that as more cationic lipids are used during particle formation, more protein is cytoplasmically delivered as quantified by the fold-increase in median splitGFP fluorescence over the negative control. The right panel shows the relationship between the cationic lipid amounts used and viability, as well as the percent of cells gated as splitGFP positive. Dashed line indicates 90% of the cell population. Data are means±SEM of n=4 biological replicates.

FIG. 33 shows Ritux-(pAbBD-D₃₀/E₃₀-S11)₂ delivery with Lipofectamine RNAiMax. 80,000 HEK293T splitGFP(1-10) cells were seeded onto each well of a 48 well plate in 180 μL media at 37° C. for 12-16 hours. Lipid nanoparticles were formed by incubating the indicated amount of cationic lipid with Ritux-(pAbBD-D₃₀/E₃₀-S11)₂ (500 nM final concentration in well) in OptiMEM (20 μL final volume, pH 7.4) at 25° C. for 10 min. The lipid nanoparticles were then added to the cells and incubated for 6 hours at 37° C. before determining the amount of splitGFP complementation by flow cytometry or viability by LDH assay. Negative controls undergo the same procedure, but with 500 nM Ritux-(pAbBD-S11)₂ protein. The left panel shows a representative flow cytometry histogram of splitGFP fluorescence. For each cationic lipid amount used, the fold-increase in median splitGFP fluorescence as well as the percentage of splitGFP positive cells are indicated. The middle panel shows that as more cationic lipids are used during particle formation, more protein is cytoplasmically delivered as quantified by the fold-increase in median splitGFP fluorescence over the negative control. The right panel shows the relationship between the cationic lipid amounts used and viability, as well as the percent of cells gated as splitGFP positive. Dashed line indicates 90% of the cell population. Data are means±SEM of n=4 biological replicates.

FIG. 34 shows Ritux-(pAbBD-Dₓ/Eₓ-S11)₂ delivery with Lipofectamine RNAiMax. 500 nM Ritux-(pAbBD-Dₓ/Eₓ-S11)₂ with 5, 10, 15, 20, 25, or 30 repeats of aspartic acid (D) or glutamic acid (E) was delivered into HEK293T splitGFP(1-10) cells as previously described with 1 μL or 2 μL Lipofectamine RNAiMax. Under these conditions, cell viability remained ≥90%. Flow cytometry analysis was then used to determine the fold-increase in median splitGFP fluorescence as well as the percent of cells gated splitGFP positive. For both poly-aspartic acid and poly-glutamic acid repeats, cytoplasmic delivery increased with repeat length. There were no significant differences in delivery efficiency between poly-aspartic acid and poly-glutamic acid repeats. Maximal delivery with Lipofectamine RNAiMax was lower than that of with Lipofectamine 2000.

FIG. 35 shows Ritux-(pAbBD-D₂₅/E₂₅-S11)₂ delivery with Lipofectamine 3000. 80,000 HEK293T splitGFP(1-10) cells were seeded onto each well of a 48 well plate in 180 μL media at 37° C. for 12-16 hours. Lipid nanoparticles were formed by incubating the indicated amount of cationic lipid with Ritux-(pAbBD-D₂₅/E₂₅-S11)₂ (500 nM final concentration in well) in OptiMEM (20 μL final volume, pH 7.4) at 25° C. for 10 min. The lipid nanoparticles were then added to the cells and incubated for 6 hours at 37° C. before determining the amount of splitGFP complementation by flow cytometry or viability by LDH assay. Negative controls undergo the same procedure, but with 500 nM Ritux-(pAbBD-S11)₂ protein. The left panel shows a representative flow cytometry histogram of splitGFP fluorescence. For each cationic lipid amount used, the fold-increase in median splitGFP fluorescence, as well as the percentage of splitGFP positive cells are indicated. The middle panel shows that as more cationic lipids are used during particle formation, more protein is cytoplasmically delivered as quantified by the fold-increase in median splitGFP fluorescence over the negative control. The right panel shows the relationship between the cationic lipid amounts used and viability, as well as the percent of cells gated as splitGFP positive. Dashed line indicates 90% of the cell population. Data are means±SEM of n=3 biological replicates.

FIGS. 36A-36I show cytoplasmic IgG delivery in A549 and HT1080 splitGFP(1-10) cells. FIGS. 36A-36C show 500 nM Ritux-(pAbBD-D₂₅-S11)₂ (FIGS. 36A, 36C), Ritux-(pAbBD-E₂₅-S11)₂ (FIGS. 36B, 36C), or Ritux-(pAbBD-S11)₂ (negative control) was complexed with 1 to 3 μl of Lipo 2000 and added to A549 splitGFP(1-10) cells for 6 h. Afterwards, splitGFP fluorescence was determined by flow cytometry (FIGS. 36A, 36B) or live-cell fluorescence microscopy (FIG. 36C). For flow cytometry, the left panel shows a representative histogram of splitGFP fluorescence. Flow cytometry data were quantified as the percent of cells splitGFP-positive (middle panel) and the fold-increase in median splitGFP fluorescence over negative control (right panel). The dotted line indicates either 90% of the cell population (middle panel) or no increase in fluorescence (right panel). Viability was determined with the LDH assay. FIGS. 36D-36F show the same cytoplasmic IgG delivery as for FIG. 36A-36C, but with Lipo RNAiMax. FIG. 36G-36I) show the same cytoplasmic IgG delivery as for FIGS. 36A-36C, but with Lipo RNAiMax and HT1080 splitGFP(1-10) cells. Data are means±s.e.m., n=4, **p<0.01 ***p<0.001 (one-sided one sample t-test of log-ratios).

FIGS. 37A-37B show MRP1 Calcein and Doxorubicin Export Assays. FIG. 37A shows that in the calcein export assay, cells will first be incubated with calcein-AM, a non-fluorescent membrane permeable calcein analog. Intracellular esterases cleave calcein-AM to calcein, which is not only fluorescent, but also accumulates intracellularly since it is membrane impermeable. Cells with high MRP1 activity will rapidly export calcein whereas MRP1 inhibition with MK571, a small molecule inhibitor, or QCRL-3 will result in calcein fluorescence retention. FIG. 37B shows MRP1 overexpressing cells are resistant to doxorubicin, a MRP1 substrates, but their doxorubicin sensitivity will be restored upon MRP1 inhibition.

FIG. 38 shows cytoplasmic QCRL3 can inhibit endogenous MRP1 in HEK293T cells. Left panel: QCRL3-(pAbBD-S11)₂ and mIgG₂ₐ-(pAbBD-S11)₂ (isotype control) were directly delivered cytoplasmically into HEK293T splitGFP(1-10) cells via electroporation. Afterwards, the cells were incubated at 37° C. for 6 hours to allow for splitGFP complementation and then analyzed by flow cytometry. Electroporating with increasing concentrations of both IgGs resulted in increasing cytoplasmic delivery as quantified by fold-increase in median splitGFP fluorescence. Right panel: QCRL3-(pAbBD-S11)₂ and mIgG₂ₐ-(pAbBD-S11)₂ were directly delivered cytoplasmically into HEK293T cells via electroporation. Then cells were incubated at 37° C. for 6 hours and then loaded with 0.1 μM calcein-AM at 37° C. for 30 minutes. Afterwards, fresh media was added to the cells and they were allowed to export calcein at 37° C. for 12 hours. Flow cytometry shows that cytoplasmic delivery of QCRL3-(pAbBD-S11)₂ via electroporation, but not mIgG₂ₐ-(pAbBD-S11)₂, causes HEK293T cells to retain calcein relative to mock-electroporated HEK293T cells due to QCRL3-mediated inhibition of endogenously expressed MRP1. Data are mean±SEM of n=3 biological replicates.

FIGS. 39A-39C show cytoplasmic QCRL3 delivery inhibits MRP1 calcein-export. FIG. 39A shows representative flow cytometry histograms of calcein fluorescence after 16 h of export in calcein-loaded HT1080 cells treated with 20 μM MK571, 500 nM QCRL3-(pAbBD-D₂₅-S11)₂, 500 nM cytosolically delivered mIgG2a-(pAbBD-D₂₅-S11)₂, or 500 nM cytosolically delivered QCRL3-(pAbBD-D₂₅-S11)₂. FIG. 39B shows calcein-efflux assay quantification across HEK293T, HT1080, and A549 cell lines. Only QCRL3 delivery and MK571 treatment resulted in calcein fluorescence retention. Data are mean±s.e.m., n=4, ***p<0.001 (one-sided one sample t-test of log-ratios). FIG. 39C is the cytoplasmic delivery same as for FIG. 39A, but in calcein-loaded A549 cells treated with cytosolic delivery of 500 nM QCRL3 with or without photocrosslinking to pAbBD-D₂₅-S11. Calcein fluorescence retention is only seen with photocrosslinked QCRL3, indicating that photocrosslinking is necessary for delivery.

FIGS. 40A-40B show cytoplasmically delivered QCRL3 sensitizes A549 cells to doxorubicin (FIG. 40A) and vincristine (FIG. 40B). 70,000 A549 were seeded onto each well of a 24 well plate in 360 μl of media at 37° C. for 12-16 hours. Lipid nanoparticles were formed by incubating 4 μl Lipofectamine 2000 with mIgG2a-(pAbBD-D₂₅-S11)₂ or QCRL3-(pAbBD-D₂₅-S11)₂ (500 nM final concentration in well) in OptiMEM (40 μl final volume, pH 7.4) at 25° C. for 10 min. The lipid nanoparticles were then added to the cells and incubated for 6 hours at 37° C. Then, the cells were trypsinized and 5,000 cells (doxorubicin) or 2,500 cells (vincristine) were reseeded onto each well of a 96 well plate in 100 μl media with the indicated concentration of doxorubicin or vincristine. After 48 hours for doxorubicin or 72 hours for vincristine, cell viability was determined by the MTT assay. For A549 cells only and 20 μM MK571 treatment conditions, 5,000 (doxorubicin) or 2,500 (vincristine) A549 cells were seeded into each well of a 96-well flat bottom tissue culture plate containing serial dilutions of either doxorubicin or vincristine in 100 μl media with or without 20 μM MK571. Data are mean±s.e.m, n=4.

FIG. 41 shows Ritux-(pAbBD-D₂₅/E₂₅-S11)₂ delivery with Lipofectamine CRISPRMax. 80,000 HEK293T splitGFP(1-10) cells were seeded onto each well of a 48 well plate in 180 μL media at 37° C. for 12-16 hours. Lipid nanoparticles were formed by incubating the indicated amount of cationic lipid with Ritux-(pAbBD-D₂₅/E₂₅-S11)₂ (500 nM final concentration in well) in OptiMEM (20 μL final volume, pH 7.4) at 25° C. for 10 min. The lipid nanoparticles were then added to the cells and incubated for 6 hours at 37° C. before determining the amount of splitGFP complementation by flow cytometry or viability by LDH assay. Negative controls undergo the same procedure, but with 500 nM Ritux-(pAbBD-S11)₂ protein. The left panel shows a representative flow cytometry histogram of splitGFP fluorescence. For each cationic lipid amount used, the fold-increase in median splitGFP fluorescence, as well as the percentage of splitGFP positive cells are indicated. The middle panel shows that as more cationic lipids are used during particle formation, more protein is cytoplasmically delivered as quantified by the fold-increase in median splitGFP fluorescence over the negative control. The right panel shows the relationship between the cationic lipid amounts used and viability, as well as the percent of cells gated as splitGFP positive. Dashed line indicates either no increase in fluorescence (middle panels) or 90% of the cell population (right panels). Data are means±SEM of n=4 biological replicates.

FIG. 42 shows Ritux-(pAbBD-D₂₅/E₂₅-S11)₂ delivery with Lipofectamine MessengerMax. 80,000 HEK293T splitGFP(1-10) cells were seeded onto each well of a 48 well plate in 180 μL media at 37° C. for 12-16 hours. Lipid nanoparticles were formed by incubating the indicated amount of cationic lipid with Ritux-(pAbBD-D₂₅/E₂₅-S11)₂ (500 nM final concentration in well) in OptiMEM (20 μL final volume, pH 7.4) at 25° C. for 10 min. The lipid nanoparticles were then added to the cells and incubated for 6 hours at 37° C. before determining the amount of splitGFP complementation by flow cytometry or viability by LDH assay. Negative controls undergo the same procedure, but with 500 nM Ritux-(pAbBD-S11)₂ protein. The left panel shows a representative flow cytometry histogram of splitGFP fluorescence. For each cationic lipid amount used, the fold-increase in median splitGFP fluorescence, as well as the percentage of splitGFP positive cells are indicated. The middle panel shows that as more cationic lipids are used during particle formation, more protein is cytoplasmically delivered as quantified by the fold-increase in median splitGFP fluorescence over the negative control. The right panel shows the relationship between the cationic lipid amounts used and viability, as well as the percent of cells gated as splitGFP positive. Dashed line indicates either no increase in fluorescence (middle panels) or 90% of the cell population (right panels). Data are means±SEM of n=4 biological replicates.

FIGS. 43A-43B show delivery scope for IgGs of different species and isotypes. 35,000 A549 splitGFP(1-10) cells were seeded onto each well of a 48 well plate in 180 μL media at 37° C. for 12-16 hours. Lipid nanoparticles were formed by incubating 2 μl Lipofectamine 2000 with IgG-(pAbBD-D₂₅-S11)₂ (500 nM final concentration in well) in OptiMEM (20 μL final volume, pH 7.4) at 25° C. for 10 min. Human IgG1 (hIgG1), hIgG2, mouse IgG2a (mIgG2a), mIgG2b, mIgG3, rat IgG2c (rIgG2c), and rabbit IgG (rabIgG) were tested for compatibility with the cytoplasmic delivery strategy. The lipid nanoparticles were then added to the cells and incubated for 6 hours at 37° C. before determining the amount of splitGFP complementation by flow cytometry or viability by LDH assay. Negative controls undergo the same procedure, but with 500 nM IgG-(pAbBD-S11)₂ protein. Flow cytometry data were quantified as the (FIG. 43A) percentage of cells splitGFP-positive and the (FIG. 43B) fold-increase in median splitGFP fluorescence over negative control. The dashed line indicates no increase in fluorescence. Data are means±SEM of n=4 biological replicates. **p<0.01, ***p<0.001.

FIG. 44 shows the relationship between Ritux-(pAbBD-D₂₅-S11)₂ delivery concentration and delivery efficiency. 35,000 A549 splitGFP(1-10) cells were seeded onto each well of a 48 well plate in 180 μL media at 37° C. for 12-16 hours. Lipid nanoparticles were formed by incubating 2 μl Lipofectamine 2000 with the indicated concentration of Ritux-(pAbBD-D₂₅-S11)₂ in OptiMEM (20 μL final volume, pH 7.4) at 25° C. for 10 min. The lipid nanoparticles were then added to the cells and incubated for 6 hours at 37° C. before determining the amount of splitGFP complementation by flow cytometry or viability by LDH assay. Negative controls undergo the same procedure, but with 500 nM Ritux-(pAbBD-S11)₂ protein. The left panel shows a representative flow cytometry histogram of splitGFP fluorescence. Flow cytometry data were quantified as the percent of cells splitGFP-positive (middle panel) and the fold-increase in median splitGFP fluorescence over negative control (right panel). The dotted line indicates either 90% of the cell population (middle panel) or no increase in fluorescence (right panel). Dashed line indicates either no increase in fluorescence (middle panels) or 90% of the cell population (right panels). Data are means±SEM of n=4 biological replicates. **p<0.01, ***p<0.001.

FIG. 45 shows the relationship between Ritux-(pAbBD-E₂₅-S11)₂ delivery concentration and delivery efficiency. 35,000 A549 splitGFP(1-10) cells were seeded onto each well of a 48 well plate in 180 μL media at 37° C. for 12-16 hours. Lipid nanoparticles were formed by incubating 21 Lipofectamine 2000 with the indicated concentration of Ritux-(pAbBD-E₂₅-S11)₂ in OptiMEM (20 μL final volume, pH 7.4) at 25° C. for 10 min. The lipid nanoparticles were then added to the cells and incubated for 6 hours at 37° C. before determining the amount of splitGFP complementation by flow cytometry or viability by LDH assay. Negative controls undergo the same procedure, but with 500 nM Ritux-(pAbBD-S11)₂ protein. The left panel shows a representative flow cytometry histogram of splitGFP fluorescence. Flow cytometry data were quantified as the percent of cells splitGFP-positive (middle panel) and the fold-increase in median splitGFP fluorescence over negative control (right panel). The dotted line indicates either 90% of the cell population (middle panel) or no increase in fluorescence (right panel). Dashed line indicates either no increase in fluorescence (middle panels) or 90% of the cell population (right panels). Data are means±SEM of n=4 biological replicates. **p<0.01, ***p<0.001.

FIGS. 46A-46D show cytosolic anti-RelA IgG delivery inhibits NFκB. FIG. 46A shows a schematic of NFκB inhibition. Anti-RelA IgGs inhibit NFκB transcriptional activity by preventing its nuclear translocation following TNFα stimulation. FIGS. 46B-46C show representative immunofluorescence images (FIG. 46B) and quantification (FIG. 46C) of RelA nuclear translocation following delivery of the indicated 150 nM IgG-(pAbBD-D25-S11)₂ antibody and TNFα treatment. Only delivery of anti-RelA IgGs reduced RelA nuclear translocation. Data are mean±s.e.m, n=3, ***p<0.001 (one-way ANOVA). In FIG. 46D A549 cells were transiently transfected with a NFκB-driven firefly luciferase reporter plasmid. NFκB transcriptional activity was detected by luminescence following delivery of the indicated 150 nM IgG-(pAbBD-D25-S11)₂ antibody and TNFα treatment. Only delivery of anti-RelA IgGs inhibited NFκB transcriptional activity. Data are mean±s.e.m, n=3, *p<0.05 **p<0.01 ***p<0.001 (one-way ANOVA).

FIGS. 47A-47E show RelA immunofluorescence quantification and are related to FIGS. 46A-46D. FIGS. 47A-47E show representative immunofluorescence images of A549 cells with or without TNFα stimulation are shown without protein delivery (FIG. 47A) or with 150 nM mIgG3-(pAbBD-D25-S11)₂ (anti-RelA NLS isotype control) (FIG. 47B), anti-RelA NLS IgG-(pAbBD-D25-S11)₂ (FIG. 47C), rabIgG-(pAbBD-D25-S11)₂ (anti-RelA C-term isotype control) (FIG. 47D), or anti-RelA C-term IgG-(pAbBD-D25-S11)₂ (FIG. 47E) delivered with 2 μl Lipo RNAiMax. CellProfiler was used for automated image analysis. The DAPI channel was used for nuclear segmentation whereas the CellMask Red channel was used for cellular segmentation. For each cell, the nuclear RelA fluorescence intensity was normalized to the cellular RelA fluorescence intensity. At least 5 image sets were taken for each biological replicate. Histograms of normalized nuclear RelA fluorescence are shown for one biological replicate for each delivery condition. 50% nuclear RelA was used as a cutoff for denoting a cell as having nuclear RelA.

FIG. 48 shows cytoplasmic delivery of proteins besides pAbBD and IgGs. Lipid nanoparticles formed with Lipofectamine 2000 and 500 nM anti-Taq affibody, anti-GFP nanobody, DARPinK27, or Omomyc with either no ApP or the indicated ApP in OptiMEM, were added to A549 splitGFP(1-10) cells and incubated for 6 hours at 37° C. before determining the amount of splitGFP complementation by flow cytometry.

FIG. 49 shows cytoplasmic delivery of DARPinK27 can inhibit KRas-G12C signaling in A549 cells.

FIGS. 50A-50B respectively show a schematic of a pAbBD-S11 fusion protein conjugated to an oligonucleotide and flow cytometry histograms of splitGFP fluorescence after incubation of A549 splitGFP(1-10) cells with lipid nanoparticles formed by incubating 2 μl Lipofectamine 2000 with 500 nM of the following pAbBD-S11-oligo conjugates: pAbBD-S11-oligo, pAbBD-D25-S11, or pAbBD-E25-S11 compared to histograms of Lipo only and pAbBD-S11 only.

FIG. 51 shows live fluorescence microscopy photos of A549 splitGFP(1-10) cells incubated with lipid nanoparticles formed by incubating 2 μl Lipofectamine 2000 with pAbBD-S11-oligonucleotide 500 nM pAbBD-S11-oligo, pAbBD-D25-S11, or pAbBD-E25-S11 in OptiMEM at 25° C. for 10 min. Lipo only and pAbBD-S11 labeled at the c-terminus with a DBCO, complexed with Lipo, were used as negative controls.

FIGS. 52A-52B respectively show a light activated site-specific conjugate of an IgG with a pAbBD-S11 fusion protein conjugated to an oligonucleotide, and representative flow cytometry histograms of splitGFP fluorescence after incubation of A549 splitGFP(1-10) cells with lipid nanoparticles formed by incubating 21 Lipofectamine 2000 with 500 nM Ritux-(pAbBD-S11-oligo)₂, Ritux-(pAbBD-D25-S11)₂, or Ritux-(pAbBD-E25-S11)₂ in OptiMEM. Negative controls underwent the same procedure, but with 500 nM Ritux-(pAbBD-S11)₂ protein or Ritux only.

FIG. 53 shows live fluorescence microscopy photos of A549 splitGFP(1-10) cells after incubation with lipid nanoparticles formed by incubating the following conjugates complexed with lipofectamine 2000: Ritux-(pAbBD-S11-DBCO)2, Ritux-(pAbBD-S11-oligo)₂, Ritux-(pAbBD-D25-S11)₂, or Ritux-(pAbBD-E25-S11)₂. Lipo only and Ritux only were used as negative controls.

### DETAILED DESCRIPTION OF THE INVENTION

The subject matter here may be understood more readily by reference to the following detailed description which forms part of this disclosure. It is to be understood that this invention is not limited to the specific products, methods, conditions or parameters described or shown here, and that the terminology used here is for the purpose of describing certain embodiments by way of example only and is not intended to be limiting of the claimed invention.

Unless otherwise defined herein, scientific and technical terms used in connection with this application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In this disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

Embodiments of the invention provide compositions and methods for cytoplasmic delivery of antibodies and other proteins. Specifically, provided herein are compositions having an anionic polypeptide, an anionic polymer, or an anionic nucleic acid and a cationic transfection agent, facilitating the cytoplasmic delivery of an antibody or a protein.

In one aspect, the antibody or other protein to be cytoplasmically delivered is fused to the anionic polypeptide, an anionic polymer, or an anionic nucleic acid. In another aspect, the anionic polypeptide, anionic polymer, or anionic nucleic acid is chemically conjugated to the antibody or other protein to be cytoplasmically delivered. In another aspect, the anionic polypeptide, anionic polymer, or anionic nucleic acid is enzymatically conjugated or ligated to the antibody or other protein to be cytoplasmically delivered. In another aspect there is a linker between the antibody or other protein to be cytoplasmically delivered and the anionic polypeptide, anionic polymer, or anionic nucleic acid.

Surprisingly and unexpectedly, the inventors of this application developed a novel, one-step bioconjugation strategy that allows the site-specific and covalent attachment of anionic polypeptides (ApP) to an antibody. The inventors of this application have shown, using common transfection agents (e.g., Lipofectamine 2000) that antibodies can be delivered into cells with a transfection efficiency of >60% at sub-micromolar concentrations and with minimal cytotoxicity. This is significantly more efficient than any non-mechanical technique that has been reported to date. The modular nature of this approach not only allows for any ‘off-the-shelf’ antibody to be easily swapped into compositions of the invention, but also preserves the binding affinity of the antibody variable region.

In one aspect, provided herein are compositions comprising: an antibody or other protein; an anionic polypeptide, an anionic polymer or an anionic nucleic acid; and a cationic transfection agent. In some embodiments, the compositions comprise: an antibody; an anionic polypeptide; and a cationic transfection agent.

The term “anionic polypeptide” refers to a polypeptide that has an anionic or a negative charge at physiologic pH. The anionic polypeptide may include a plurality of negatively charged amino acid residues or unnatural amino acid residues. The term “anionic polymer” refers to a polymer that has an anionic or a negative charge at physiologic pH. The term “anionic nucleic acid” refers to a nucleic acid that has an anionic or a negative charge at physiologic pH.

In some embodiments, the anionic polypeptide may include a plurality of “repeats” of negatively charged amino acid residues. For example, at least 20% of residues in the anionic polypeptide are repeats of negatively charged amino acid residues. In some embodiments, the anionic polypeptide may have a net charge of less than −5, less than −10, less than −20, less than −30, less than −40, less than −50, less than −100, less than −200, less than −300, less than −400, or less than −500.

Examples of negatively charged amino acid residues are well known in the art. In one embodiment, the negatively charged amino acid residue is aspartic acid. In a particular embodiment, the anionic polypeptide comprises a plurality of aspartic acid residues.

In some embodiments, the negatively charged amino acid residue is glutamic acid. In some embodiments, the anionic polypeptide comprises a plurality of glutamic acid residues.

In some embodiments, the negatively charged amino acid residue is an unnatural amino acid. For example, the anionic polypeptide comprises a plurality of negatively charged unnatural amino acid residues.

In some embodiments, the negatively charged amino acid is a glutamic acid, aspartic acid, or negatively charged unnatural amino acid. In some embodiments, the anionic polypeptide comprises a plurality of glutamic acid, aspartic acid, and negatively charged unnatural amino acid residues. In some embodiments, the anionic polypeptide comprises glutamic acid, aspartic acid residues, and negatively charged unnatural amino acids.

The number of repeats of the negatively charged amino acid residues range from about 2 to about 50, about 10 to about 40, about 20 to about 30, or about 25 to about 30.

In another aspect, the invention provides compositions comprising: a protein; an anionic nucleic acid; and a cationic transfection agent, wherein presence of the anionic nucleic acid and the cationic transfection agent in the composition facilitate cytoplasmic delivery of the protein. In some embodiments, the anionic nucleic acid is used in the composition as an alternative to an anionic polypeptide. In particular embodiments, the protein is operably linked to the anionic nucleic acid. In certain embodiments, the protein is a single chain protein. In some embodiments, the protein is operably linked to or comprises a photoreactive amino acid group. In an embodiment, the photoreactive amino acid is benzoylphenylalanine (BPA). In some embodiments, the antibody binding domain is operably linked to or comprises a photoreactive amino acid group. In some embodiments, the anionic nucleic acid is operably linked, ligated, conjugated or fused to an antibody. In certain embodiments, the anionic nucleic acid is operably linked, ligated, conjugated or fused to an AbBD.

In another embodiment, the cationic transfection agent is a nano-carrier. In an embodiment, the cationic transfection agent is an ionizable carrier. In certain embodiments, the ionizable carrier includes an ionizable-lipid, polymer, or combination thereof. In some embodiments, the ionizable carrier is an ionizable lipid-like nanoparticle. In a particular embodiment, the compositions comprising: a protein; an anionic nucleic acid; and a cationic transfection agent further comprise an agent that induces protein degradation of a target protein.

In some embodiments, the antibody binds to the target protein. In some embodiments, the anionic nucleic acid is operably linked, ligated, conjugated or fused to an antibody. In particular embodiments, the anionic nucleic acid is operably linked, ligated, conjugated or fused to an AbBD.

In various embodiments, the agent comprises a domain for targeted degradation. In some embodiments, the protein is a single chain protein. In certain embodiments, the compositions further comprise an agent that modifies the function of a target protein. In various embodiments, the compositions further comprise an agent that induces nuclear, cytoplasmic, membrane or membrane-associated proteins to be sorted into compartments where they are inactive or degraded. In particular embodiments, the single chain protein is a single chain antibody, a single chain antigen-binding fragment (scFab) or a single chain Fv (scFv). In an embodiment, the single chain protein is a single chain targeting ligand. In some embodiments, the single chain targeting ligand is an affibody, a nanobody, an antibody mimetic or a peptide. In certain embodiments, the affibody is an anti-Taq affibody. In various embodiments, the nanobody is an anti-GFP nanobody. In particular embodiments, the antibody mimetic is a genetically engineered designed ankyrin repeat protein (DARPin). In some embodiments, the peptide comprises Omomycin.

Cationic transfection agents are well known in the art. Any suitable cationic transfection agent known in the art can be used. In one embodiment, the cationic transfection agent is an ionizable carrier, for example, an ionizable-lipid, polymer, or lipid-like molecule.

In one embodiment, the cationic transfection agent is a cationic lipid. The term “cationic lipid” refers to a lipid which has a cationic, or positive charge at physiologic pH. Cationic lipids can take a variety of forms including, but not limited to, liposomes or micelles. Cationic lipids useful for certain aspects of this disclosure are known in the art, and, generally comprise both polar and non-polar domains, and bind to polyanions. Cationic lipids have been used in the art to deliver molecules to cells (see, e.g., U.S. Pat. Nos. 5,855,910; 5,851,548; 5,830,430; 5,780,053; 5,767,099; 8,569,256; 8,691,750; 8,748,667; 8,758,810; 8,759, 104; 8,771,728; Lewis et al. 1996. Proc. Natl. Acad. Sci. 93:3176; Hope et al. 1998. Molecular Membrane Biology, 15:1, each of which is incorporated by reference herein in its entirety).

Examples of cationic lipids include, but are not limited to, Lipofectin (a combination of DOTMA and DOPE), Lipofectase, LIPOFECTAMINE® (e.g., LIPOFECTAMINE® 2000, LIPOFECT AMINE® 3000, LIPOFECTAMINE® RNAiMAX, LIPOFECTAMINE® LTX, LIPOFECTAMINE® MessengerMAX™), LIPOFECTAMINE® CRISPRMax™ Cas9 Transfection Reagent, Invivofectamine, SAINT-RED (Synvolux Therapeutics, Groningen Netherlands), DOPE, Cytofectin (Gilead Sciences, Foster City, Calif.), and Eufectins (JBL, San Luis Obispo, Calif.). Exemplary cationic liposomes can be made from N-[1-(2,3-dioleoloxy)-propyl]-N,N,N-trimethylammonium chloride (DOTMA), N-[1-(2,3-dioleoloxy)-propyl]-N,N,N-trimethylammonium methylsulfate (DOTAP), 3P-[N-(N′,N′-dimethylaminoethane)carbamoyl]cholesterol (DC-Choi), 2,3,-dioleyloxy-N-[2(sperminecarboxamido)ethyl]-N,N-dimethyl-1-propanaminium trifluoroacetate (DOSPA), 1,2-dimyristyloxypropyl-3-dimethyl-hydroxyethyl ammonium bromide; and dimethyldioctadecylammonium bromide (DDAB).

In other embodiments, the cationic transfection agent is a cationic polymer. The term “cationic polymer,” as used herein, refers to a polymer having a net positive charge. Cationic polymers are well known in the art, and include those described in Samal et al., Cationic polymers and their therapeutic potential. Chem Soc Rev. 2012 Nov. 7; 41(21):7147-94; in published U.S. patent applications US2014/0141487, US2014/0141094, US2014/0044793, US2014/0018404, US2014/0005269, and US2013/0344117; and in U.S. Pat. Nos. 8,709,466; 8,728,526; 8,759,103; and 8,790,664; the entire contents of each are incorporated herein by reference. Exemplary cationic polymers include, but are not limited to, polyallylamine (PAH); polyethyleneimine (PEI); poly(L-lysine) (PLL); poly(L-arginine) (PLA); polyvinylamine homo- or copolymer; a poly(vinylbenzyl-tri-Ci-C₄-alkylammonium salt); polyethylenimine, polyamidoamine (PAMAM) starburst dendrimers, In vivo-jetPEI, TransIT-QR, a polymer of an aliphatic or araliphatic dihalide and an aliphatic N,N,N′,N′-tetra-Ci-C₄-alkyl-alkylenediamine; a poly(vinylpyridin) or poly(vinylpyridinium salt); a poly(N,N-diallyl-N,N-di-Ci-C₄-alkyl-ammoniumhalide); a homo- or co-polymer of a quaternized di-Ci-C₄-alkyl-aminoethyl acrylate or methacrylate; POLYQUAD™; and a polyaminoamide.

Suitable cationic lipids, lipid-like materials and cationic polymers are disclosed herein, and additional suitable lipids and lipid-like materials are known in the art (see, e.g., those described in Akinc et al., Nature Biotechnology 26, 561-569 (2008), the entire contents of which is incorporated herein by reference).

In one embodiment, the cationic transfection agent is a nano-carrier. Any suitable nano-carrier known in the art can be used. In one embodiment, the cationic transfection agent is an ionizable lipid-like nanoparticle. For example, the ionizable lipid comprises a polyamine core structure reacted with an alkyl epoxide.

In some embodiments that nano-carrier is pegylated or coated with a material that increases solubility, increases biocompatibility, reduces opsonization, and/or extends circulation time. The nanoparticle may be further modified with a targeting ligand that confers specificity for a cell surface receptor.

In some embodiments, compositions of the invention include an agent that induces protein degradation.

Agents that can induce protein degradation may be a protein, polypeptide, small molecule, or nucleic acid that can induce intracellular degradation of a protein or protein complexes that the agent is associated with, either covalently linked or non-covalently bound to. Proteins and polypeptides that can induce target protein degradation include, but are not limited to, degrons, destabilizing domains such as FKBP L106P (Banaszynski et al. 2006. Cell. 126:995) or ecDHFR destabilizing domains (Iwamoto et al. 2010. Chem. Biol. 17:981), protein domains or fragments that can recruit the proteasome to target proteins such as fragments of ornithine decarboxylase (Renicke et al. 2013. Chem. Biol. 20:619), proteases or other enzymes that can expose degradation signals in target proteins, proteins or peptides that are recognition sequences for protein degradation machinery, and ubiquitin ligation-associated proteins. Ubiquitin ligation-associated proteins refer to the entire or fragments of E1 ubiquitin-activating enzymes, E3 ubiquitin-conjugating enzymes, E3 ubiquitin-protein ligases, or other proteins that associate with E1, E2, or E3 enzymes. Examples of ubiquitin ligation-associated proteins that can induce targeted protein degradation includes, but are not limited to, the promiscuous E3 ligase CHIP (Portnoff et al. 2014. J. Biol. Chem. 289:7844), the IAA17 degron paired with the TIR1 protein (Nishimura et al. 2009. Nat. Methods. 6:917), Slmb as well as its F-box domain fragment (Caussinus et al. 2012. Nat. Struct. Mol. Biol. 19:117), the E3 ubiquitin ligase adaptor SPOP (Shin et al. 2015. Sci. Rep. 5:14269), and the VHL protein as well as its fragments (Fulcher et al. 2016. Open Biol. 6:160255).

Nucleic acids that can induce target protein degradation include, but are not limited to, aptamers that can recruit the proteasome or ubiquitin ligation-associated proteins to target proteins.

Small molecules that can induce target protein degradation include, but are not limited to, molecules that work via hydrophobic tagging such as molecules containing an adamantyl group (Neklesa et al. 2011. Nat. Chem. Biol. 7:538) or Boc3Arg groups (Long et al. 2012. Chem. Biol. 19:629), as well as molecules that can recruit target proteins to E3 ligases such as proteolysis targeting chimera (PROTACs, Sakamoto et al. 2001. Proc. Natl. Acad. Sci. U.S.A. 98:8554). Small molecule PROTACs include, but are not limited to, molecules containing nutlin-3a and nutlin derivatives that recruit target proteins to MDM2 (Schneekloth et al. 2008. Bioorg. Med. Chem. Lett. 18:5904), molecules containing bestatin and bestatin derivatives that recruit target proteins to IAP1 (Itoh et al. 2010. J. Am. Chem. Soc. 132:5820), molecules that bind and recruit target proteins to the VHL E3 Ligase (Buckley et al. 2012. J. Am. Chem. Soc. 134:4465), and molecules containing pthalimides and pthalimide derivatives which recruit target proteins to the cereblon E3 ligase (Lu et al. 2015. Chem. Biol. 22:755).

The agents may have their degradative capability engineered to be under control of light, small molecules, or temperature.

In one embodiment, the compositions of the invention include an agent that modifies the function of a target protein.

Agents that can modify target protein function by localization may include a protein, polypeptide, small molecule, or nucleic acid that can modify target protein function through its recruitment to a specific subcellular compartment or by inducing target protein aggregation. Target proteins may be recruited to compartments in which they are active in order to augment their function or may be recruited to compartments in which they are inactive in order to decrease target protein function. Subcellular compartments include the nucleus, lysosome, mitochondria, cytoplasm, plasma membrane, as well as any other membrane-bound or membrane-less organelle. Target proteins may be recruited to each other or to aggregation-prone proteins in order to induce target protein aggregation and modify its function.

For example, compositions of the invention may include an agent, such as a protein, polypeptide, small molecule, or nucleic acid, that induces nuclear, cytoplasmic, membrane, or membrane-associated target proteins to be sorted into compartments where they are inactive or degraded.

In one embodiment, the protein to be cytoplasmically delivered can be an affinity protein or a therapeutic protein. In some embodiments, the protein is an enzyme, transcription factor, nuclease, nucleic acid binding protein, genome editing protein, or Cas9. In some embodiments, the protein is a protein-based drug or toxin. In some embodiments, the protein is an antibody. In other embodiments, the protein is an artificial affinity protein, such as an affibody, Affitin, Carbohydrate binding module, DARPin, knottin, monobody, nanobody, or other scaffold known in the art. In a particular embodiment, the affibody is an anti-Taq affibody. In another embodiment, the anti-Taq affibody inhibits Taq polymerase activity. In another embodiment, the nanobody is an anti-GFP nanobody. In certain embodiments, the DARPin is the antibody mimetic DARPinK27. In another embodiment, DARPinK27, inhibits KRas activity. In some embodiments, the protein to be cytoplasmically delivered is Omomyc, which is a mini-protein derived from the basic helix-loop-helix (bHLH) domain of Myc. Omomyc is Myc/Max oncogene inhibitor having mutations in the leucine zipper to improve dimerization.

The term “antibody” is used herein in the broadest sense and specifically covers monoclonal antibodies, polyclonal antibodies, dimers, multimers, multispecific antibodies (e.g., bispecific antibodies), and antibody fragments, so long as they exhibit the desired biological activity. Antibodies may be murine, human, humanized, chimeric, or derived from other species. An antibody is a protein generated by the immune system that is able to recognize and bind to a specific antigen. A target antigen generally has numerous binding sites, also called epitopes, recognized by CDRs on multiple antibodies. Each antibody that specifically binds to a different epitope has a different structure. Thus, an antigen may have more than one corresponding antibody. An antibody includes a full-length immunoglobulin or an immunologically active portion of a full-length immunoglobulin, i.e., a molecule that contains an antigen binding site that immunospecifically binds an antigen of a target of interest or part thereof, such targets include, but are not limited to, cancer cell or cells that produce autoimmune antibodies associated with an autoimmune disease.

In another aspect, a composition of the invention comprises an antibody-binding domain (AbBD) operably linked to a photoreactive amino acid (e.g., benzoylphenylalanine), creating a photoreactive antibody binding domain (pAbBD), wherein said domain is operably linked to an antibody or a fragment thereof. “Antibody fragments” comprise a portion of a full length antibody, generally the antigen binding or variable region thereof. Examples of antibody fragments include Fab, Fab′, F(ab′)₂, and Fv fragments; diabodies; linear antibodies; fragments produced by a Fab expression library, anti-idiotypic (anti-Id) antibodies, CDR (complementary determining region), and epitope-binding fragments of the above which immunospecifically bind to cancer cell antigens, viral antigens or microbial antigens, single-chain antibody molecules; and multispecific antibodies formed from antibody fragments.

In some embodiments, a small antibody-binding domain (AbBD) is used. In other embodiments, the AbBD is engineered to contain a photoreactive unnatural amino acid in its Fc-binding site. The introduction of a photoreactive amino acid allows for formation of a covalent linkage between a photoreactive antibody-binding domain (pAbBD)-anionic polypeptide (ApP) fusion protein and an antibody.

In another aspect, the anionic polypeptide is fused to the AbBD or pAbBD. In another aspect, the anionic polypeptide, anionic polymer, or anionic nucleic acid is chemically conjugated to the AbBD or pAbBD. In another aspect, the anionic polypeptide, anionic polymer, or anionic nucleic acid is enzymatically conjugated or ligated to the AbBD or pAbBD. In another aspect, there is a linker between the anionic polypeptide, anionic polymer, or anionic nucleic acid and the AbBD or pAbBD.

The AbBD or pAbBD is able to bind to both heavy chains of an antibody, thereby creating highly negatively charged antibodies that can be efficiently packaged with cationic lipids, polymers, and or lipid-like materials and delivered into cells using similar approaches to that are used for gene delivery.

In some embodiments, the AbBD is engineered to contain a chemical moiety that allows for proximity-induced antibody conjugation (e.g. reactive halide, aryl ketone, Michael acceptor, aryl isothiocyanate, aryl carbamate side chains, or other moeities known in the art), enabling site-specific covalent bond formation without UV or chemical treatment.

In another aspect, provided herein is an AbBD comprising a protein, such as a Protein G HTB1 domain, Protein Z domain, Protein A, Protein G, Protein L, Protein LG, Protein LA, Protein A/G, or an Fc-binding peptide, such as Fc-III, Fc-III-4C, APAR, PAM, FcBP-2, RRGW, KHRFNKD, or sub-domains thereof having at least one amino acid or amino acid modifications that are adapted to specifically bind and crosslink to an immunoglobulin. In another aspect, provided herein is a conjugate molecule or an adapter comprising a first antibody binding domain (AbBD) fused to a second antibody binding domains (AbBD), wherein the first AbBD has one or more amino acids or amino acid modifications that are adapted to specifically bind and crosslink to a first immunoglobulin and wherein the second AbBD has one or more amino acids or amino acid modifications that are adapted to specifically bind and crosslink to a second immunoglobulin. In a particular embodiment, the immunoglobulin is IgG.

For example, the antibody binding domain (AbBD) crosslinks to an immunoglobulin Fc region. In another example, the antibody binding domain (AbBD) crosslinks to an immunoglobulin Fab region.

As used herein, the term “Fc domain” encompasses the constant region of an immunoglobulin molecule. The Fc region of an antibody interacts with a number of Fc receptors and ligands, imparting an array of important functional capabilities referred to as effector functions, as described herein. For IgG the Fc region comprises Ig domains CH2 and CH3. An important family of Fc receptors for the IgG isotype are the Fc gamma receptors (FcγRs). These receptors mediate communication between antibodies and the cellular arm of the immune system.

As used herein, the term “Fab domain” encompasses the region of an antibody that binds to antigens. The Fab region is composed of one constant and one variable domain of each of the heavy and the light chains.

As used herein, the term “immunoglobulin G” or “IgG” refers to a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, IgG3. As used herein, the term “modified immunoglobulin G” refers to a molecule that is derived from an antibody of the “G” class. For example, the antibody is a protein consisting of one or more polypeptides substantially encoded by all or part of the recognized immunoglobulin genes. The recognized immunoglobulin genes, for example in humans, include the kappa (κ) lambda (λ) and heavy chain genetic loci, which together comprise the myriad variable region genes, and the constant region genes mu (μ) delta (δ), gamma (γ), sigma (σ) and alpha (α) which encode the IgM, IgD, IgG, IgE, and IgA isotypes or classes, respectively. The term “antibody” is meant to include full-length antibodies, and may refer to a natural antibody from any organism, an engineered antibody, or an antibody generated recombinantly for experimental, therapeutic, diagnostic or other purposes. Furthermore, full-length antibodies comprise conjugates as described and exemplified herein. Antibodies can be antagonists, agonists, neutralizing, inhibitory, or stimulatory. Specifically included within the definition of “antibody” are full-length antibodies described and exemplified herein. By “full length antibody” herein is meant the structure that constitutes the natural biological form of an antibody, including variable and constant regions.

The “variable region” of an antibody contains the antigen binding determinants of the molecule, and thus determines the specificity of an antibody for its target antigen. The variable region is so named because it is the most distinct in sequence from other antibodies within the same isotype. The majority of sequence variability occurs in the complementarity determining regions (CDRs). There are 6 CDRs total, three each per heavy and light chain, designated VH CDR1, VH CDR2, VH CDR3, VL CDR1, VL CDR2, and VL CDR3. The variable region outside of the CDRs is referred to as the framework (FR) region. Although not as diverse as the CDRs, sequence variability does occur in the FR region between different antibodies. Overall, this characteristic architecture of antibodies provides a stable scaffold (the FR region) upon which substantial antigen binding diversity (the CDRs) can be explored by the immune system to obtain specificity for a broad array of antigens.

In addition, antibodies may exist in a variety of other forms including, for example, Fv, Fab, and (Fab′)₂, as well as bi-functional (i.e. bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., “Immunology”, Benjamin, N.Y., 2^{nd} ed. (1984), and Hunkapiller and Hood, Nature, 323, 15-16 (1986)).

The term “epitope” refers to a region of an antigen that binds to the antibody or antigen-binding fragment. It is the region of an antigen recognized by a first antibody, where the binding of the first antibody to the region prevents binding of a second antibody or other bivalent molecule to the region. The region encompasses a particular core sequence or sequences selectively recognized by a class of antibodies. In general, epitopes are comprised by local surface structures that can be formed by contiguous or noncontiguous amino acid sequences.

As used herein, the terms “selectively recognizes”, “selectively bind” or “selectively recognized” mean that binding of the antibody, antigen-binding fragment or other bivalent molecule to an epitope is at least 2-fold greater, preferably 2-5 fold greater, and most preferably more than 5-fold greater than the binding of the molecule to an unrelated epitope or than the binding of an antibody, antigen-binding fragment or other bivalent molecule to the epitope, as determined by techniques known in the art and described herein, such as, for example, ELISA or cold displacement assays.

As used herein, the term “antibody” encompasses the structure that constitutes the natural biological form of an antibody. In most mammals, including humans, and mice, this form is a tetramer and consists of two identical pairs of two immunoglobulin chains, each pair having one light and one heavy chain, each light chain comprising immunoglobulin domains V_{L} and C_{L}, and each heavy chain comprising immunoglobulin domains V_{H}, Cγ1, Cγ2, and Cγ3. In each pair, the light and heavy chain variable regions (V_{L} and V_{H}) are together responsible for binding to an antigen, and the constant regions (C_{L}, Cγ1, Cγ2, and Cγ3, particularly Cγ2, and Cγ3) are responsible for antibody effector functions. In some mammals, for example in camels and llamas, full-length antibodies may consist of only two heavy chains, each heavy chain comprising immunoglobulin domains V_{H}, Cγ2, and Cγ3. By “immunoglobulin (Ig)” is meant a protein having one or more polypeptides substantially encoded by immunoglobulin genes. Immunoglobulins include, but are not limited to, antibodies. Immunoglobulins may have a number of structural forms, including but not limited to full-length antibodies, antibody fragments, and individual immunoglobulin domains including but not limited to V_{H}, Cγ1, Cγ2, Cγ3, V_{L}, and C_{L}.

Depending on the amino acid sequence of the constant domain of their heavy chains, intact antibodies can be assigned to different “classes.” There are five-major classes (isotypes) of intact antibodies: IgA, IgD, IgE, IgG, and IgM, and several of these may be further divided into “subclasses”, e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known to one skilled in the art.

In one embodiment, the term “antibody” or “antigen-binding fragment” respectively refer to intact molecules as well as functional fragments thereof, such as Fab, a scFv-Fc bivalent molecule, F(ab′)₂, and Fv that are capable of specifically interacting with a desired target. In some embodiments, the antigen-binding fragments comprise:

(1) Fab, the fragment which contains a monovalent antigen-binding fragment of an antibody molecule, which can be produced by digestion of whole antibody with the enzyme papain to yield an intact light chain and a portion of one heavy chain;

(2) Fab′, the fragment of an antibody molecule that can be obtained by treating whole antibody with pepsin, followed by reduction, to yield an intact light chain and a portion of the heavy chain; two Fab′ fragments are obtained per antibody molecule;

(3) (Fab′)₂, the fragment of the antibody that can be obtained by treating whole antibody with the enzyme pepsin without subsequent reduction; F(ab′)2 is a dimer of two Fab′ fragments held together by two disulfide bonds;

(4) Fv, a genetically engineered fragment containing the variable region of the light chain and the variable region of the heavy chain expressed as two chains; and

(5) Single chain antibody (“SCA”), a genetically engineered molecule containing the variable region of the light chain and the variable region of the heavy chain, linked by a suitable polypeptide linker as a genetically fused single chain molecule.

(6) scFv-Fc, is produced in one embodiment, by fusing single-chain Fv (scFv) with a hinge region from an immunoglobulin (Ig) such as an IgG, and Fc regions.

In some embodiments, an antibody provided herein is a monoclonal antibody. In some embodiments, the antigen-binding fragment provided herein is a single chain Fv (scFv), a diabody, a tandem scFv, a scFv-Fc bivalent molecule, an Fab, Fab′, Fv, F(ab′)₂ or an antigen binding scaffold (e.g., affibody, monobody, anticalin, DARPin, Knottin, etc.).

As used herein, the terms “binds” or “binding” or grammatical equivalents, refer to compositions having affinity for each other. “Specific binding” is where the binding is selective between two molecules. A particular example of specific binding is that which occurs between an antibody and an antigen. Typically, specific binding can be distinguished from non-specific when the dissociation constant (K_{D}) is less than about 1×10⁻⁵ M or less than about 1×10⁻⁶ M or 1×10⁻⁷ M. Specific binding can be detected, for example, by ELISA, immunoprecipitation, coprecipitation, with or without chemical crosslinking, two-hybrid assays and the like. Appropriate controls can be used to distinguish between “specific” and “non-specific” binding.

In some embodiments, an antibody or antigen-binding fragment provided herein comprises a modification. For example, the modification minimizes conformational changes during the shift from displayed to secreted forms of the antibody or antigen-binding fragment. It is to be understood by a skilled artisan that the modification can be a modification known in the art to impart a functional property that would not otherwise be present if it were not for the presence of the modification. Encompassed are antibodies which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques including, but not limited, to specific chemical cleavage by cyanogen bromide, trypsin, chymotrypsin, papain, V8 protease, NaBH4, acetylation, formylation, oxidation, reduction, metabolic synthesis in the presence of tunicamycin, etc.

In some embodiments, the modification is a N-terminus modification. In some embodiments, the modification is a C-terminus modification. In some embodiments, the modification is an N-terminus biotinylation. In some embodiments, the modification is an C-terminus biotinylation. In some embodiments, the secretable form of the antibody or antigen-binding fragment comprises an N-terminal modification that allows binding to an Immunoglobulin (Ig) hinge region. some embodiments, the Ig hinge region is from but is not limited to, an IgA hinge region. In some embodiments, the secretable form of the antibody or antigen-binding fragment comprises an N-terminal modification that allows binding to an enzymatically biotinylatable site. In some embodiments, the secretable form of the antibody or antigen-binding fragment comprises an C-terminal modification that allows binding to an enzymatically biotinylatable site. In some embodiments, biotinylation of said site functionilizes the site to bind to any surface coated with streptavidin, avidin, avidin-derived moieties, or a secondary reagent.

It will be appreciated that the term “modification” can encompass an amino acid modification, such as an amino acid substitution, insertion, and/or deletion in a polypeptide sequence.

In one embodiment, a variety of radioactive isotopes are available for the production of radioconjugate antibodies and can be of use in the methods and compositions provided herein. Examples include, but are not limited to, At²¹¹, I¹³¹, I¹²⁵, Y⁹⁰, Re¹⁸⁶, Re¹⁸⁸, Sm¹⁵³, Bi²¹², P³², and radioactive isotopes of Lu.

In some embodiments, enzymatically active toxin or fragments thereof that can be used in the compositions and methods provided herein include, but are not limited to, diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

A chemotherapeutic or other cytotoxic agent may be conjugated to the antibody or protein described herein. In one aspect, the chemotherapeutic or cytotoxic agent may be a prodrug. The term “prodrug” refers to a precursor or derivative form of a pharmaceutically active substance that is less cytotoxic to tumor cells compared to the parent drug and is capable of being enzymatically activated or converted into the more active parent form. See, for example Wilman, 1986, Biochemical Society Transactions, 615th Meeting Belfast, 14:375-382; and Stella et al., “Prodrugs: A Chemical Approach to Targeted Drug Delivery,” Directed Drug Delivery, Borchardt et al., (ed.): 247-267, Humana Press, 1985. The prodrugs that may find use with the compositions and methods as provided herein include but are not limited to phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use with the antibodies and Fc fusions of the compositions and methods as provided herein include but are not limited to any of the aforementioned chemotherapeutic.

In one embodiment, a combination of a recombinant protein with the biological active agents specified above, i.e., a cytokine, an enzyme, a chemokine, a radioisotope, an enzymatically active toxin, or a chemotherapeutic agent can be used.

In one embodiment, a variety of other therapeutic agents may find use for administration with the antibodies and conjugates of the compositions and methods provided herein. In one embodiment, the conjugate comprising an antibody is administered with an anti-angiogenic agent. As used herein, the term “anti-angiogenic agent” refers to a compound that blocks, or interferes to some degree, the development of blood vessels. The anti-angiogenic factor may, for instance, be a small molecule or a protein, for example an antibody, Fc fusion, or cytokine, that binds to a growth factor or growth factor receptor involved in promoting angiogenesis. In an alternate embodiment, the composition is administered with a therapeutic agent that induces or enhances adaptive immune response. In an alternate embodiment, the conjugate is administered with a tyrosine kinase inhibitor. The term “tyrosine kinase inhibitor” refers to a molecule that inhibits to some extent tyrosine kinase activity of a tyrosine kinase as known in the art.

In one embodiment, the compositions provided herein may be used for various therapeutic or diagnostic purposes. In one embodiment, the conjugates are administered to a subject to treat an antibody-related disorder. In another embodiment, the conjugate proteins are administered to a subject with an inflammatory disease. In another embodiment, the conjugate proteins are administered to a subject with an auto-immune disease. In another embodiment, the conjugate proteins are administered to a subject with a neurological disorder. In another embodiment, the conjugate proteins are administered to a subject to treat a tumor or a cancer. A “subject” for the purposes of the compositions and methods provided herein includes humans and other animals, preferably mammals and most preferably humans. Thus the conjugates provided herein have both human therapy and veterinary applications. In another embodiment the subject is a mammal, and in yet another embodiment the subject is human. By “condition” or “disease” herein are meant a disorder that may be ameliorated by the administration of a pharmaceutical composition comprising the conjugate of the compositions and methods provided herein. Antibody related disorders include but are not limited to autoimmune diseases, immunological diseases, infectious diseases, inflammatory diseases, neurological diseases, and oncological and neoplastic diseases including cancer.

In some embodiments, an antibody or protein of the invention may be labeled or conjugated with an imaging agent. Imaging agents may include a radionuclide, fluorescent dye, magnetic resonance contrast agent, CT contrast agent, or any other agent capable of providing contrast on an acquired image.

In another embodiment, provided herein are nucleic acid constructs encoding the fusion proteins or conjugate components provided herein. The term “nucleic acid” refers to polynucleotide or to oligonucleotides such as deoxyribonucleic acid (DNA), and, where appropriate, ribonucleic acid (RNA) or mimetic thereof. The term should also be understood to include, as equivalents, analogs of either RNA or DNA made from nucleotide analogs, and, as applicable to the embodiment being described, single (sense or antisense) and double-stranded polynucleotides. This term includes oligonucleotides composed of naturally occurring nucleobases, sugars and covalent internucleoside (backbone) linkages as well as oligonucleotides having non-naturally-occurring portions, which function similarly. Such modified or substituted oligonucleotides are often preferred over native forms because of desirable properties such as, for example, enhanced cellular uptake, enhanced affinity for nucleic acid target and increased stability in the presence of nucleases.

In one embodiment, provided herein are primers used for amplification and construction of the vectors and nucleic acids provided herein. It is to be understood by a skilled artisan that other primers can be used or designed to arrive at the vectors, nucleic acids and conjugates provided herein.

In one embodiment, provided herein is a vector comprising the nucleic acid encoding for the fusion protein or conjugate components provided herein. In another embodiment, the vector comprises nucleic acid encoding the recombinant protein, polypeptides, peptides, antibodies, and recombinant fusions provided herein.

In another embodiment, the nucleic acid can be expressed in a variety of different systems, in vitro and in vivo, according to the desired purpose. For example, a nucleic acid can be inserted into an expression vector, introduced into a desired host, and cultured under conditions effective to achieve expression of a polypeptide coded for by the nucleic acid. Effective conditions include any culture conditions which are suitable for achieving production of the polypeptide by the host cell, including effective temperatures, pH, medusa, additives to the media in which the host cell is cultured (e.g., additives which amplify or induce expression such as butyrate, or methotrexate if the coding nucleic acid is adjacent to a dhfr gene), cycloheximide, cell densities, culture dishes, etc. In another embodiment, a nucleic acid can be introduced into the cell by any effective method including, e.g., naked DNA, calcium phosphate precipitation, electroporation, injection, DEAE-Dextran mediated transfection, fusion with liposomes, association with agents which enhance its uptake into cells, viral transfection. A cell into which the nucleic acid provided herein has been introduced is a transformed host cell. The nucleic acid can be extrachromosomal or integrated into a chromosome(s) of the host cell. It can be stable or transient. An expression vector is selected for its compatibility with the host cell. Host cells include, mammalian cells (e.g., COS-7, CV1, BHK, CHO, HeLa, LTK, NIH 3T3, 293, PAE, human, human fibroblast, human primary tumor cells, testes cells), insect cells, such as Sf9 (S. frugiperda) and Drosophila, bacteria, such as E. coli, Streptococcus, Bacillus, yeast, such as S. cerevisiae (e.g., cdc mutants, cdc25, cell cycle and division mutants, such as ATCC Nos. 42563, 46572, 46573, 44822, 44823, 46590, 46605, 42414, 44824, 42029, 44825, 44826, 42413, 200626, 28199, 200238, 74155, 44827, 74154, 74099, 201204, 48894, 42564, 201487, 48893, 28199, 38598, 201391, 201392), fungal cells, plant cells, embryonic stem cells (e.g., mammalian, such as mouse or human), fibroblasts, muscle cells, neuronal cells, etc. Expression control sequences are similarly selected for host compatibility and a desired purpose, e.g., high copy number, high amounts, induction, amplification, controlled expression. Other sequences which can be employed include enhancers such as from SV40, CMV, RSV, inducible promoters, cell-type specific elements, or sequences which allow selective or specific cell expression. Promoters that can be used to drive its expression, include, e.g., the endogenous promoter, promoters of other genes in the cell signal transduction pathway, MMTV, SV40, trp, lac, tac, or T7 promoters for bacterial hosts; or alpha factor, alcohol oxidase, or PGH promoters for yeast.

In one embodiment, reporter genes may be incorporated into expression constructs to facilitate identification of transcribed products. For example, reporter genes used are selected from β-galactosidase, chloramphenicol acetyl transferase, luciferase or a fluorescent protein.

In one embodiment, the conjugates are purified or isolated after expression. Proteins may be isolated or purified in a variety of ways known to those skilled in the art. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation, dialysis, and chromatofocusing techniques. Ultrafiltration and diafiltration techniques, in conjunction with protein concentration, are also useful. As is well known in the art, a variety of natural proteins bind Fc and antibodies, and these proteins can find use in the present invention for purification of conjugates. For example, the bacterial proteins A and G bind to the Fc region. Likewise, the bacterial protein L binds to the Fab region of some antibodies, as of course does the antibody's target antigen. Purification can often be enabled by a particular fusion partner. For example, proteins may be purified using glutathione resin if a GST fusion is employed, Ni⁺² affinity chromatography if a His-tag is employed, or immobilized anti-flag antibody if a flag-tag is used. The degree of purification necessary will vary depending on the screen or use of the conjugates. In some instances no purification is necessary. For example in one embodiment, if the conjugates are secreted, screening may take place directly from the media. As is well known in the art, some methods of selection do not involve purification of proteins. Thus, for example, if a library of conjugates is made into a phage display library, protein purification may not be performed.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, when referring to a measurable value such as an amount, a temporal duration, a concentration, and the like, may encompass variations of ±20% or ±10%, more preferably ±5%, even more preferably ±1%, and still more preferably ±0.1% from the specified value, as such variations are appropriate to perform the disclosed methods.

Described herein are techniques for the rapid production of antibody conjugates using full-length IgG. These techniques generally do not require any genetic manipulation of the IgG. Any off the shelf IgGs can be used to make the antibodies.

IgGs are site-specifically modified using photoreactive antibody binding domains. Antibody binding domains (AbBDs) include Protein A, Protein G, Protein L, Protein LG, Protein LA, Protein A/G, CD4 and their subdomains, e.g., B1 domain of Protein G, engineered subdomains, e.g., Protein Z, HTB1, or an Fc-binding peptide, such as Fc-III, Fc-III-4C, APAR, PAM, FcBP-2, RRGW, KHRFNKD, or sub-domains thereof.

The term “Protein Z,” as used here, refers to the Z domain based on the B domain of Staphylococcal aureus Protein A. The amino acid sequence of wild-type Protein Z is: VDNKFNKEQQNAFYEILHLPNLNEEQRNAFIQSLKDDPSQSANLLAEAKKLNDAQAP KMRM (SEQ ID NO: 1). Photoreactive Protein Z includes those where an amino acid in protein Z has been replaced with benzoylphenylalanine (BPA), such as F13BPA and F5BPA (see underlined amino acids in bold in SEQ ID NO: 1). Examples of other BPA-containing mutants of Protein Z include, for example, but are not limited to, Q32BPA, K35BPA, N28BPA, N23BPA, and L17BPA. Examples of Protein Z variants or mutants include, F5I, such as F5I K35BPA. The Protein Z amino acid sequence may also include homologous, variant, and fragment sequences having Z domain function. In some embodiments, the Protein Z amino acid sequence may include an amino acid sequence which is 60, 65, 70, 75, 80, 85, 90, 95, or 99% identity to the sequence set forth in SEQ ID NO: 22.

The term “Protein G,” as used herein, refers to a B1 domain based of Streptococcal Protein G. Preferably, the Protein G is a hypothermophilic variant of a B1 domain based of Streptococcal Protein G. The amino acid sequence of Protein G preferably is: MTFKLIINGKTLKGEITIEAVDAAEAEKIFKQYANDYGIDGEWTYDDATKTFTVTE (SEQ ID NO: 2). Nine Protein G variants were successfully designed and expressed, each having an Fc-facing amino acid substituted by BPA: V21, A24, K28, 129, K31, Q32, D40, E42, W42 (see underlined amino acids in bold in SEQ ID NO: 2). Two variants, A24BPA and K28BPA, allowed ˜100% of all human IgG subtypes to be labeled. The Protein G amino acid sequence may also include homologous, variant, and fragment sequences having B1 domain function. In some embodiments, the Protein G amino acid sequence may include an amino acid sequence which is 60, 65, 70, 75, 80, 85, 90, 95, or 99% identity to the sequence set forth in SEQ ID NO: 2.

In some embodiments, one or more photoreactive groups, e.g., benzophenone, are introduced into the AbBDs. These can be incorporated into the AbBDs during translation (e.g., benzoylphenylalanine, BPA) using non-natural amino acid incorporation, synthetically during peptide synthesis, or the AbBDs can be post-modified with a photocrosslinker (e.g., 4-(N-Maleimido)benzophenone). In this case, a cysteine is introduced into the AbBD at the location where a benzophenone is desired. BPA as a photoreactive crosslinker has several favorable properties. Specifically, BPA's benzophenone group can be activated by long wavelength UV light (365 nm), which is not harmful to antibodies or other proteins. In addition, even after being UV excited to its triplet state, benzophenone can relax back to its unreactive ground state if there are no abstractable hydrogen atoms in close proximity. This allows photoreactive proteins to be produced and handled in ambient light conditions with low risk of photobleaching. However, other photoreactive crosslinkers can also be used, including those that possess aryl azides, diazirines, or other photoreactive moieties known in the art.

There are many techniques, known in the art for linking molecules. A variety of linkers be used in the compositions and methods provided herein to generate conjugates or fusions.

The term “linker”, “linker sequence”, “spacer”, “tethering sequence” or grammatical equivalents thereof refer to a molecule or group of molecules (such as a monomer or polymer) that connects two molecules and often serves to place the two molecules in a preferred configuration. A number of strategies may be used to covalently link molecules together. These include, but are not limited to polypeptide linkages between N- and C-terminus of proteins or protein domains, linkage via disulfide bonds, and linkage via chemical cross-linking reagents. In one aspect of this embodiment, the linker is a peptide bond, generated by recombinant techniques or peptide synthesis. In another embodiment the linker is a cysteine linker. In yet another embodiment it is a multi-cysteine linker. Choosing a suitable linker for a specific case where two polypeptide chains are to be connected depends on various parameters, including but not limited to the nature of the two polypeptide chains (e.g., whether they naturally oligomerize), the distance between the N- and the C-termini to be connected if known, and/or the stability of the linker towards proteolysis and oxidation. Furthermore, the linker may contain amino acid residues that provide flexibility. Thus, the linker peptide may predominantly include the following amino acid residues: Gly, Ser, Ala, or Thr. The linker peptide should have a length that is adequate to link two molecules in such a way that they assume the correct conformation relative to one another so that they retain the desired activity. Suitable lengths for this purpose include at least one and not more than 30 amino acid residues. In one embodiment, the linker is from about 1 to 30 amino acids in length. In another embodiment, the linker is from about 1 to 15 amino acids in length. In addition, the amino acid residues selected for inclusion in the linker peptide should exhibit properties that do not interfere significantly with the activity of the polypeptide. Thus, the linker peptide on the whole should not exhibit a charge that would be inconsistent with the activity of the polypeptide, or interfere with internal folding, or form bonds or other interactions with amino acid residues in one or more of the monomers that would seriously impede the binding of receptor monomer domains. Useful linkers include glycine-serine polymers, glycine-alanine polymers, alanine-serine polymers, and other flexible linkers such as the tether for the shaker potassium channel, and a large variety of other flexible linkers, as will be appreciated by those in the art. Suitable linkers may also be identified by screening databases of known three-dimensional structures for naturally occurring motifs that can bridge the gap between two polypeptide chains. In one embodiment, the linker is not immunogenic when administered in a human subject. Thus, linkers may be chosen such that they have low immunogenicity or are thought to have low immunogenicity. Another way of obtaining a suitable linker is by optimizing a simple linker, e.g., (Gly₄Ser)ₙ, through random mutagenesis. Alternatively, once a suitable polypeptide linker is defined, additional linker polypeptides can be created to select amino acids that more optimally interact with the domains being linked. Other types of linkers that may be used in the compositions and methods provided herein include artificial polypeptide linkers and inteins. In another embodiment, disulfide bonds are designed to link the two molecules. In another embodiment, linkers are chemical cross-linking agents. For example, a variety of bifunctional protein coupling agents may be used, including but not limited to N-succinimidyl-3-(2-pyridyldithiol) propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). In another embodiment, chemical linkers may enable chelation of an isotope. For example, Carbon-14-labeled 1-isothiocyanatobenzyl-3-methyldiethylene triaminepentaacetic acid (MX-DTPA) is an exemplary chelating agent for conjugation of radionucleotide to the antibody. In another embodiment, the linker may be cleavable. For example, an acid-labile linker, peptidase-sensitive linker, dimethyl linker or disulfide-containing linker (Chari et al., 1992, Cancer Research 52: 127-131) may be used. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers, that is may find use to link the components of the conjugates of the compositions and methods provided herein. In another embodiment, a cleavable linker may facilitate release of the cytotoxic drug in the cell.

In one aspect, the invention provides biological linking modules. These are fused in frame with an antibody or protein to be cytoplasmically delivered, the AbBDs, and/or anionic polypeptides (ApP) at the N- or C-terminus. In some embodiments, the AbBDs, are fused in frame with an anionic polypeptides (ApP) at the N- or C-terminus.

SpyCatcher and SpyTag. AbBD or ApP can be fused to SpyCatcher, SpyTag, or a combination thereof. See Zakeri et al., “Peptide tag forming a rapid covalent bond to a protein, through engineering a bacterial adhesin” PNAS (2012) vol. 109 no. 12, pgs. E690-E697, doi: 10.1073/pnas.1115485109, which is hereby incorporated by reference in its entirety.

Split inteins (or other intein-based systems). AbBD or ApP can be fused to the split intein.

Heterodimeric proteins that have an affinity for each other (e.g., c-fos and c-jun, leucine zippers, peptide velcro, etc.) can also be used.

Dock-and-lock. This system involves two docking proteins, which can be fused to the AbBDs or ApPs. These proteins bring together the two molecules. Then a third peptide is used to covalently link the two docking proteins together.

Sortase. Sortase substrates (e.g., LPXTG and an N-terminal glycine) can be fused to the AbBDs or ApPs.

In another aspect, provided herein are chemical linking modules. The antibody or protein to be cytoplasmically delivered, AbBDs or ApPs are modified at their N- or C-terminus with various chemical moieties that can be used to link them together or to other proteins, peptides, nucleic acids, polymers, or small molecules (including but not limited to drugs or imaging agents).

Click chemistries. The antibody or protein to be cytoplasmically delivered, AbBD or ApP can be modified with an azide, an alkyne or constrained alkyne (e.g., ADIBO or DBCO). Other popular click chemistries exist (e.g., tetrazine and TCO). Click chemistries can be incorporated using various techniques, e.g., intein-mediated expressed protein ligation, sortase, sortase-tag expressed protein ligation, non-natural amino acid incorporation, maleimide chemistry, carbodiimide chemistry, NHS chemistry, aldehyde chemistry, chemoenzymatic approaches (e.g., lipoic acid ligase, formylglycine), etc.

In one aspect, the invention provides oligonucleotides. Click chemistries or conventional chemistries are used to attach oligonucleotides (e.g., complementary oligonucleotides) to the antibody or protein to be cytoplasmically delivered, AbBDs or ApPs.

In one example, AbBDs with complementary linking modules (e.g., SpyCatcher and SpyTag) are covalently linked to IgG upon exposure to long UV light (typically long wavelength UV light). The two complementary AbBD-IgG conjugates are then mixed together to form the bispecific antibody.

In other embodiments, a single construct with two photoreactive AbBDs fused together are used to make bispecific antibodies. For example, photoreactive AbBDs with unique specificity for different IgG isotypes are fused. Therefore, if it is desirable to link together two IgGs with two distinct subclasses, it is not necessary to use a linking module; rather AbBDs that are directly fused together can be used.

Similarly, in other embodiments, IgG homodimers are prepared using AbBDs that are fused together and do not require a linking module.

In one aspect, provided herein is a method for sensitizing a tumor cell to a chemotherapeutic agent, the method comprising administering to cytoplasm of the tumor cell: (i) a conjugate comprising an antibody binding domain (AbBD) operably linked, ligated or fused to an anionic polypeptide comprising a plurality of negatively charged amino acid residues or (ii) a cell recombinantly expressing the conjugate of (i). In some embodiments, the antibody binding domain is operably linked to or comprises a photoreactive amino acid group. In a particular embodiment, the photoreactive amino acid is benzoylphenylalanine. In another embodiment, at least 20% of residues in the anionic polypeptide are negatively charged amino acid or unnatural amino acid residues. In certain embodiments, the negatively charged amino acid residues are aspartic acid residues, glutamic acid residues, or a combination of aspartic acid residues and glutamic acid residues. In some embodiments, the number of the plurality of amino acid residues ranges from about 2 to about 50, from about 10 to about 40, from about 20 to about 30, or from about 25 to about 30. In some embodiments, the conjugate comprises an anionic nucleic acid as an alternative to an anionic polypeptide. In some embodiments, the anionic nucleic acid is operably linked, ligated, conjugated or fused to an antibody. In particular embodiments, the anionic nucleic acid is operably linked, ligated, conjugated or fused to an AbBD.

In an embodiment, the conjugate or the cell recombinantly expressing the conjugate further comprises or is mixed with or complexed with a cationic transfection agent. In another embodiment, the conjugate or the cell recombinantly expressing the conjugate further comprises an agent that modifies the function of a target protein. In some embodiments, the conjugate or the cell recombinantly expressing the conjugate further comprises an agent that induces nuclear, cytoplasmic, membrane or membrane-associated proteins to be sorted into compartments where they are inactive or degraded. In another embodiment, the conjugate or the cell recombinantly expressing the conjugate further comprises an agent that induces a protein degradation. In an embodiment, the agent that induces the protein degradation comprises a domain for targeted degradation. In some embodiments, the AbBD comprises an AbBD of anti-human multidrug resistance-associated protein 1 (MRP1) monoclonal antibody QCRL3. In an embodiment, the AbBD of anti-human MRP1 binds to conformation-dependent internal epitope of human MRP1, and the epitope comprising amino acids 617-932 of human MRP1. In another embodiment of the herein provided method, the chemotherapeutic agent is doxorubicin or vincristine. In certain embodiments, the tumor cell is a multidrug resistant (MDR) tumor cell. In other embodiments, the MDR tumor cell is a human non-P-glycoprotein MDR tumor cell.

In another aspect, provided herein is a method for sensitizing a tumor cell to a chemotherapeutic agent, the method comprising administering to cytoplasm of the tumor cell: (i) a conjugate comprising protein operably linked, ligated or conjugated to an anionic nucleic acid or (ii) a cell recombinantly expressing the conjugate of (i). In some embodiments, the protein is operably linked or conjugated to the anionic nucleic acid. In an embodiment, the protein is a single chain protein, as described herein. In some embodiments, the anionic nucleic acid is operably linked, ligated, conjugated or fused to an antibody. In particular embodiments, the anionic nucleic acid is operably linked, ligated, conjugated or fused to an AbBD. In an embodiment, the antibody binding domain is operably linked to or comprises a photoreactive amino acid group. In a particular embodiment, the photoreactive amino acid is benzoylphenylalanine. In another embodiment, at least 20% of residues in the anionic polypeptide are negatively charged amino acid or unnatural amino acid residues. In certain embodiments, the negatively charged amino acid residues are aspartic acid residues, glutamic acid residues, or a combination of aspartic acid residues and glutamic acid residues. In some embodiments, the number of the plurality of amino acid residues ranges from about 2 to about 50, from about 10 to about 40, from about 20 to about 30, or from about 25 to about 30. In an embodiment, the conjugate or the cell recombinantly expressing the conjugate further comprises or is mixed with or complexed with a cationic transfection agent. In another embodiment, the conjugate or the cell recombinantly expressing the conjugate further comprises an agent that modifies the function of a target protein. In some embodiments, the conjugate or the cell recombinantly expressing the conjugate further comprises an agent that induces nuclear, cytoplasmic, membrane or membrane-associated proteins to be sorted into compartments where they are inactive or degraded. In another embodiment, the conjugate or the cell recombinantly expressing the conjugate further comprises an agent that induces a protein degradation. In an embodiment, the agent that induces the protein degradation comprises a domain for targeted degradation. In some embodiments, the AbBD comprises an AbBD of anti-human multidrug resistance-associated protein 1 (MRP1) monoclonal antibody QCRL3. In an embodiment, the AbBD of anti-human MRP1 binds to conformation-dependent internal epitope of human MRP1, and the epitope comprising amino acids 617-932 of human MRP1. In another embodiment of the herein provided method, the chemotherapeutic agent is doxorubicin or vincristine. In certain embodiments, the tumor cell is a multidrug resistant (MDR) tumor cell. In other embodiments, the MDR tumor cell is a human non-P-glycoprotein MDR tumor cell.

In another aspect, provided herein is a method for decreasing or inhibiting growth of a tumor cell, the method comprising administering to cytoplasm of the tumor cell in a subject in need thereof a composition comprising an antibody or other protein; an anionic polypeptide; and a cationic transfection agent, wherein the anionic polypeptide comprises a plurality of negatively charged amino acid residues, and wherein the presence of the anionic polypeptide and the cationic transfection agent in the composition facilitate cytoplasmic delivery of the antibody or other protein. In an embodiment, the protein is a therapeutic protein. In another embodiment, the protein is a protein-based drug or toxin. In some embodiments, the protein is an artificial affinity protein. In certain embodiments, the antibody is a bispecific antibody. In certain embodiments, the antibody is an immunoglobulin G (IgG) or a fragment thereof. In particular embodiments, the anionic polypeptide is operably linked to the antibody or other protein. In an embodiment, the polypeptide is fused to the antibody or other protein. In some embodiments, the antibody is operably linked to an antibody binding domain (AbBD), wherein the AbBD is operably linked or fused to the anionic polypeptide. In specific embodiments, the antibody binding domain is operably linked to or comprises a photoreactive amino acid group. In particular embodiments, the photoreactive amino acid is benzoylphenylalanine (BPA). In some embodiments, at least 20% of residues in the anionic polypeptide are negatively charged amino acid or unnatural amino acid residues. In another embodiment, the negatively charged amino acid residues are aspartic acid residues, glutamic acid residues, or a combination of aspartic acid residues and glutamic acid residues. In still another embodiment, the number of the plurality of amino acid residues ranges from about 2 to about 50, from about 10 to about 40, from about 20 to about 30, or from about 25 to about 30. In certain embodiments, the AbBD is operably linked or fused to an anionic nucleic acid instead of/as an alternative to the anionic polypeptide. In some embodiments, the cationic transfection agent is a nano-carrier. In some embodiments, the anionic nucleic acid is operably linked, ligated, conjugated or fused to an antibody. In particular embodiments, the anionic nucleic acid is operably linked, ligated, conjugated or fused to an AbBD.

In particular embodiments, the cationic transfection agent is an ionizable carrier. In certain embodiments, the ionizable carrier includes an ionizable-lipid, polymer, or combination thereof. In an embodiment, the ionizable carrier is an ionizable lipid-like nanoparticle. In an embodiment, the composition comprising an antibody or other protein; an anionic polypeptide; and a cationic transfection agent further comprises an agent that induces protein degradation. In some embodiments, the agent that induces protein degradation comprises a domain for targeted degradation. In another embodiment, the composition further comprises an agent that modifies the function of a target protein. In some embodiments, the composition further comprises an agent that induces nuclear, cytoplasmic, membrane or membrane-associated proteins to be sorted into compartments where they are inactive or degraded. In particular embodiments, the other protein comprises genetically engineered designed ankyrin repeat proteins (DARPins). In specific embodiments, the other protein comprises Omomycin. In another embodiment, the AbBD comprises an AbBD of anti-human multidrug resistance-associated protein 1 (MRP1) monoclonal antibody QCRL3. In some embodiments, the tumor cell is a multidrug resistant (MDR) tumor cell. In particular embodiments, the MDR tumor cell is a human non-P-glycoprotein MDR tumor cell.

In another aspect, provided herein is a method for inhibiting NF-kB transcription and/or reducing RelA nuclear translocation a cancer cell, the method comprising administering to cytoplasm of the cancer cell in a subject in need thereof a composition comprising an antibody or other protein; an anionic polypeptide; and a cationic transfection agent, wherein the anionic polypeptide comprises a plurality of negatively charged amino acid residues, and wherein the presence of the anionic polypeptide and the cationic transfection agent in the composition facilitate cytoplasmic delivery of the antibody or other protein. In an embodiment, the protein is a therapeutic protein. In some embodiments, the protein is a protein-based drug or toxin. In certain embodiments, the protein is an artificial affinity protein. In other embodiments, the antibody is a bispecific antibody. In specific embodiments, the antibody is an immunoglobulin G (IgG) or a fragment thereof. In particular embodiments, the anionic polypeptide is operably linked to the antibody or other protein. In some embodiments, the anionic polypeptide is fused to the antibody or other protein. In another embodiment, the antibody is operably linked to an antibody binding domain (AbBD), wherein the AbBD is operably linked or fused to the anionic polypeptide. In some embodiments, the AbBD is operably linked or fused to an anionic nucleic acid instead of/as an alternative to the anionic polypeptide. In particular embodiments, the antibody binding domain is operably linked to or comprises a photoreactive amino acid group. In specific embodiments, the photoreactive amino acid is benzoylphenylalanine (BPA). In another embodiment, at least 20% of residues in the anionic polypeptide are negatively charged amino acid or unnatural amino acid residues. In some embodiment, the negatively charged amino acid residues are aspartic acid residues, glutamic acid residues, or a combination of aspartic acid residues and glutamic acid residues. In an embodiment, the number of the plurality of amino acid residues ranges from about 2 to about 50, from about 10 to about 40, from about 20 to about 30, or from about 25 to about 30. In a particular embodiment, the cationic transfection agent is a nano-carrier. In an embodiment, the cationic transfection agent is an ionizable carrier. In another embodiment, the ionizable carrier includes an ionizable-lipid, polymer, or combination thereof. In some embodiments, the ionizable carrier is an ionizable lipid-like nanoparticle. In a particular embodiment, the composition comprising an antibody or other protein further comprises an agent that induces protein degradation. In another embodiment of the composition, the agent comprises a domain for targeted degradation. In still another embodiment, the composition further comprises an agent that modifies the function of a target protein. In another embodiment, further comprising an agent that induces nuclear, cytoplasmic, membrane or membrane-associated proteins to be sorted into compartments where they are inactive or degraded. In specific embodiments, the AbBD comprises an AbBD of anti-RelA antibody, wherein the antibody is an IgG.

It will be appreciated that the methods provided herein can also be used to make antibody-protein and antibody-enzyme conjugates, as well as other types of antibody-conjugates. In these cases, the second linking module is placed on the protein or enzyme that is to be linked to the IgG or AbBD-IgG conjugate, which contains the other half of the linking module, e.g., to make IgG-affibody conjugates.

Also provided herein are nucleic acids and vectors that encode the conjugates described herein. Further provided herein are cells that express the conjugates described herein.

In another aspect, the invention also provides pharmaceutical compositions.

Pharmaceutical compositions are contemplated wherein fusion conjugate or adopter of the compositions and methods provided herein and one or more therapeutically active agents are formulated. Formulations of the conjugates of the compositions and methods provided herein are prepared for storage by mixing said antibody or Fc fusion having the desired degree of purity with optional pharmaceutically acceptable carriers, excipients or stabilizers, in the form of lyophilized formulations or aqueous solutions. Acceptable carriers, excipients, or stabilizers are nontoxic to recipients at the dosages and concentrations employed, and include buffers such as phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g., Zn-protein complexes); and/or non-ionic surfactants or polyethylene glycol (PEG). In another embodiment, the pharmaceutical composition that comprises the conjugate of the compositions and methods provided herein is in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine. The formulations to be used for in vivo administration are preferably sterile. This is readily accomplished by filtration through sterile filtration membranes or other methods.

The conjugate molecules disclosed herein may also be formulated as immunoliposomes. A liposome is a small vesicle comprising various types of lipids, phospholipids and/or surfactant that is useful for delivery of a therapeutic agent to a mammal. Liposomes containing the conjugates are prepared by methods known in the art, such as described in Epstein et al., 1985, Proc Nat'l Acad Sci USA, 82:3688; Hwang et al., 1980, Proc Nat'l Acad Sci USA, 77:4030; U.S. Pat. Nos. 4,485,045; 4,544,545; and PCT WO 97/38731. Liposomes with enhanced circulation time are disclosed in U.S. Pat. No. 5,013,556. The components of the liposome are commonly arranged in a bilayer formation, similar to the lipid arrangement of biological membranes. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. A chemotherapeutic agent or other therapeutically active agent is optionally contained within the liposome (Gabizon et al., 1989, J National Cancer Inst 81:1484).

The conjugate molecules provided herein may also be entrapped in microcapsules prepared by methods including but not limited to coacervation techniques, interfacial polymerization (for example using hydroxymethylcellulose or gelatin-microcapsules, or poly-(methylmethacylate) microcapsules), colloidal drug delivery systems (for example, liposomes, albumin microspheres, microemulsions, nano-particles and nanocapsules), and macroemulsions. Such techniques are disclosed in Remington's Pharmaceutical Sciences 16th edition, Osol, A. Ed., 1980. Sustained-release preparations may be prepared. Suitable examples of sustained-release preparations include semipermeable matrices of solid hydrophobic polymer, which matrices are in the form of shaped articles, e.g., films, or microcapsules. Examples of sustained-release matrices include polyesters, hydrogels (for example poly(2-hydroxyethyl-methacrylate), or poly(vinylalcohol)), polylactides (U.S. Pat. No. 3,773,919), copolymers of L-glutamic acid and gamma ethyl-L-glutamate, non-degradable ethylene-vinyl acetate, degradable lactic acid-glycolic acid copolymers (which are injectable microspheres composed of lactic acid-glycolic acid copolymer and leuprolide acetate), and poly-D-(−)-3-hydroxybutyric acid) which is a microsphere-based delivery system composed of the desired bioactive molecule incorporated into a matrix of poly-DL-lactide-co-glycolide (PLG).

The conjugate molecules may also be linked to nanoparticle surfaces using the linking methods provided herein. In one embodiment, the nanoparticles can be used for imaging or therapeutic purposes.

Administration of the pharmaceutical composition comprising the conjugates provided herein, preferably in the form of a sterile aqueous solution, may be done in a variety of ways, including, but not limited to, orally, subcutaneously, intravenously, intranasally, intraotically, transdermally, topically (e.g., gels, salves, lotions, creams, etc.), intraperitoneally, intramuscularly, intrapulmonary, intratumoral, vaginally, parenterally, rectally, or intraocularly. As is known in the art, the pharmaceutical composition may be formulated accordingly depending upon the manner of introduction.

According to one aspect, provided herein is a method of delivering an antibody or other protein to cell cytoplasm in a subject, comprising: providing a herein provided composition of the invention; and administering said composition to said subject.

In another aspect, provided herein is a method of delivering a protein to cytoplasm of a cell in a subject, comprising: providing a herein provided composition of the invention; and administering said composition to said subject, wherein the composition comprises: a protein; an anionic nucleic acid; and a cationic transfection agent. In an embodiment, the protein is a single chain protein, as described herein. In various embodiments, the anionic nucleic acid is operably linked, ligated, conjugated or fused to an antibody. In particular embodiments, the anionic nucleic acid is operably linked, ligated, conjugated or fused to an AbBD.

According to another aspect, provided herein is a method of treating a disease or disorder in a subject, comprising: delivering a composition of the invention to cell cytoplasm in the subject. According to yet another aspect, provided herein is a method for manufacturing a composition for a cytoplasmic delivery, comprising: covalently linking, ligating, or fusing an antibody or other protein with an anionic polypeptide in order to prepare a conjugate; and mixing or complexing a cationic transfection agent with said conjugate. In one aspect, provided herein is a method for manufacturing a composition for a cytoplasmic delivery, comprising: covalently linking, ligating, or fusing a protein to an anionic nucleic acid; and mixing or complexing a cationic transfection agent with the conjugate. In some embodiments, the protein is a s single chain protein, as described herein. In various embodiments, the anionic nucleic acid is operably linked, ligated, conjugated or fused to an antibody. In particular embodiments, the anionic nucleic acid is operably linked, ligated, conjugated or fused to an AbBD.

In another aspect, the invention provides a method of treating a disease or disorder in a subject, comprising: delivering a composition described herein to cell cytoplasm in the subject.

In another aspect, the invention provides a method of manufacturing a composition for a cytoplasmic delivery, comprising: covalently linking, ligating, or fusing an antibody or other protein with an anionic polypeptide in order to prepare a conjugate; and mixing or complexing a cationic transfection agent with said conjugate.

In one aspect, the invention provides a method of manufacturing a composition for a cytoplasmic delivery, comprising: covalently linking, ligating, or fusing a protein to an anionic nucleic acid in order to prepare a conjugate; and mixing or complexing a cationic transfection agent with the conjugate. In an embodiment, the protein is a single chain protein. In some embodiments, the anionic nucleic acid is operably linked, ligated, conjugated or fused to an antibody. In particular embodiments, the anionic nucleic acid is operably linked, ligated, conjugated or fused to an AbBD.

The term “subject” refers to a mammal, including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

The following examples are presented in order to more fully illustrate the preferred embodiments of the invention. They should in no way be construed, however, as limiting the broad scope of the invention.

### EXAMPLES

### Example 1

### Cytoplasmic Delivery of Antibodies

Photoreactive antibody-binding domains (pAbBDs): Since antibodies are unable to cross cell membranes, their delivery into cells will most certainly require some sort of modification, which would likely either have to be introduced as a fusion protein or via a post-translational chemical conjugation. While made-to-order genes, advanced expression systems, and new high efficiency cloning techniques can simplify and accelerate the production of fusion proteins, the need for genetic engineering and expression of complex proteins severely limits the throughput of this approach. It also requires information on the sequence of the IgG variable region or other targeting domain (if another scaffold is used), which is rarely available. Moreover, fusions with IgG or antibody fragments may exhibit a loss of specificity, aggregation, and heterogeneity, potentially requiring months or more to optimize. Because of the time needed to make fusion proteins, it will also likely be necessary to pre-validate any antibody that is to be used, perhaps by microinjection or electroporation, to ensure that the antibody effectively inhibits the desired pathway, prior to committing to a lengthy genetic engineering process. Unfortunately, these mechanical delivery approaches are not widely available and have their own limitations, e.g., low viability, low efficiency. This creates a barrier for the widespread study of antibodies against intracellular targets, even if a methodology for cytoplasmic delivery was identified.

Clearly, a chemically-based approach to modifying antibodies post-translationally that does not require antibody engineering and cloning would offer significant advantages in regards to increasing the throughput in which antibodies can be tested against intracellular targets. In particular, it would open up the possibility of using any ‘off-the-shelf’, commercially available antibody, with no need of prior knowledge of the IgG sequence or target epitope. Moreover, the time required to prepare the antibody conjugates would be reduced from months to hours and pre-validation with mechanical delivery methods would no longer be required.

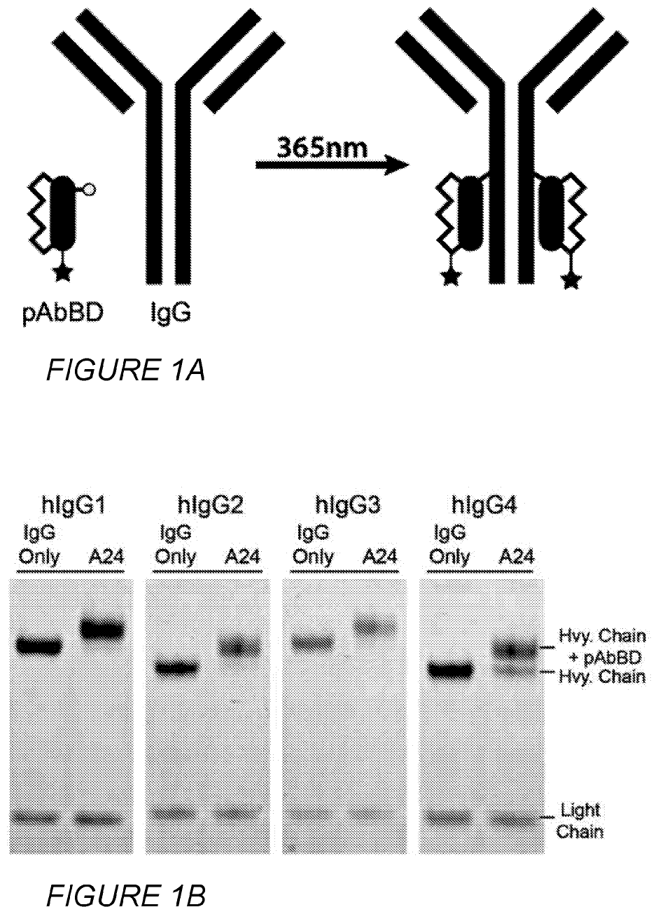

Recently, a rapid and site-specific bioconjugation technique was developed that allows for the efficient attachment of small molecules, polypeptides, proteins, or enzymes to full-length IgG. This technique uses a small antibody-binding domain that is engineered to contain a photoreactive unnatural amino acid (benzoyl-phenylalanine, BPA) in its Fc-binding site (FIG. 1). The photoreactive antibody-binding domain (pAbBD) is created from a small (˜6.5 kD), thermally stable domain of Protein G (HTB1). The introduction of a photoreactive amino acid allows for the formation of a covalent linkage between the pAbBD and IgG. The pAbBD is capable of photocrosslinking to both heavy chains of IgG from a wide range of hosts (e.g., human, mouse rat, rabbit, goat, etc.) and subclasses. The pAbBD can be fused with nearly any desired biomolecule, thus allowing for the attachment of the fused protein to IgG. The pAbBD fusions can be grown in large yields in bacterial expression systems using standard techniques. Binding of the pAbBD to Fc sites of IgG does not interfere with normal IgG binding affinity. Therefore, light-activated site-specific conjugation with pAbBDs represents a highly modular and universal approach to making IgG conjugates for cytoplasmic delivery and enables nearly any ‘off-the-shelf’ IgG to be easily swapped into this system without the need for genetic engineering.

Anionic polypeptide-IgG conjugates: If antibodies could be efficiently delivered into the cytosol of living cells, it would significantly increase the number of possible druggable targets. Antibodies can be developed to bind nearly any exposed protein epitope, with high specificity and affinity. There are a countless number of therapeutic possibilities that could be pursued if antibodies could be effectively delivered into cells, from inhibiting protein function, to driving proteins interactions, to tagging proteins for proteasomal degradation. Not surprisingly, numerous attempts have been made to deliver antibodies into cells, but a robust and efficient approach has yet to be identified.

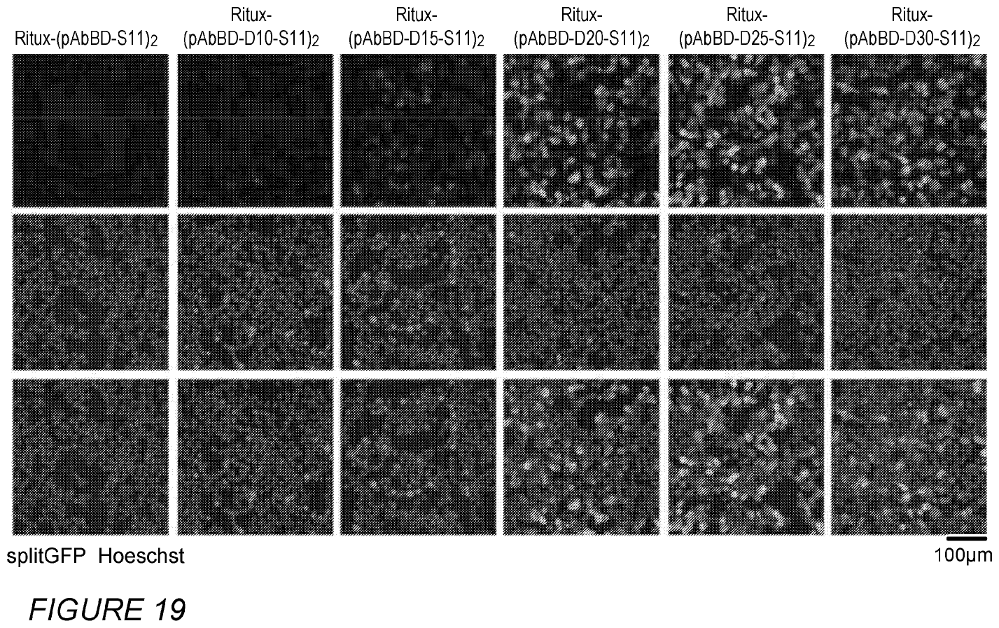

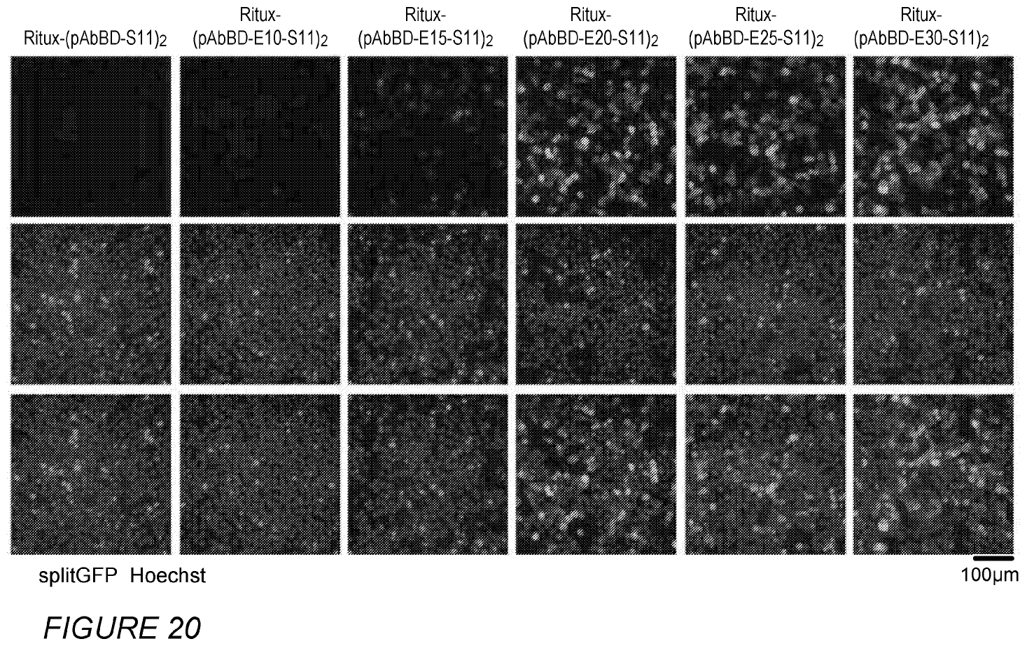

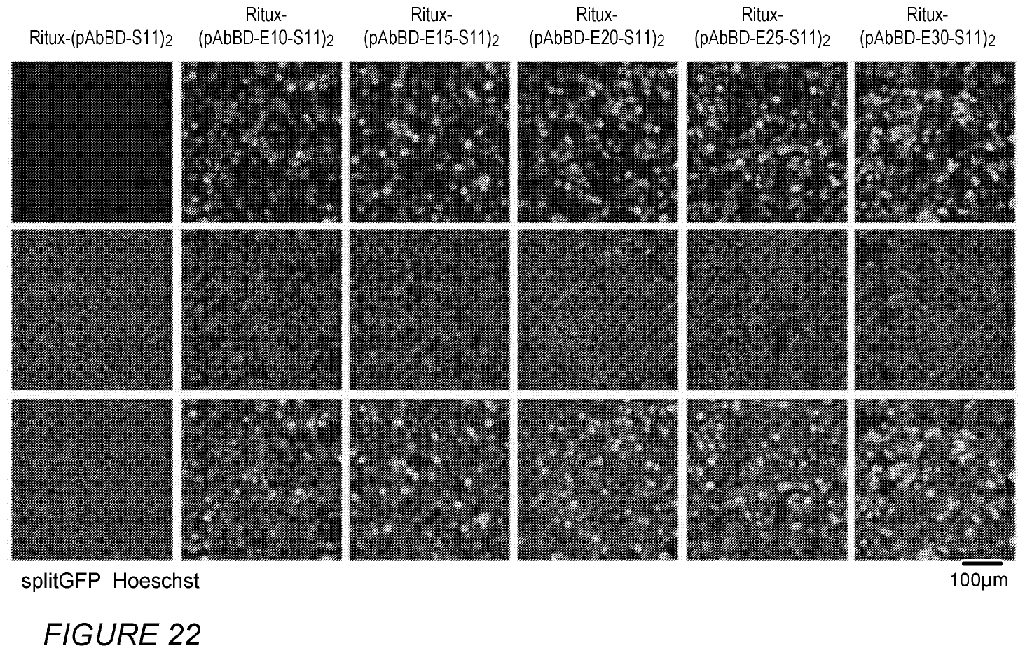

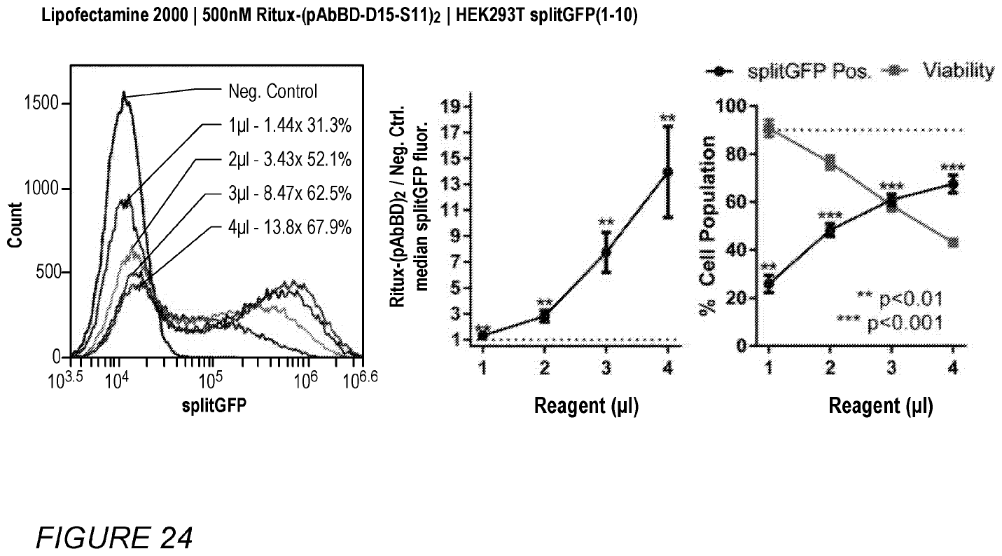

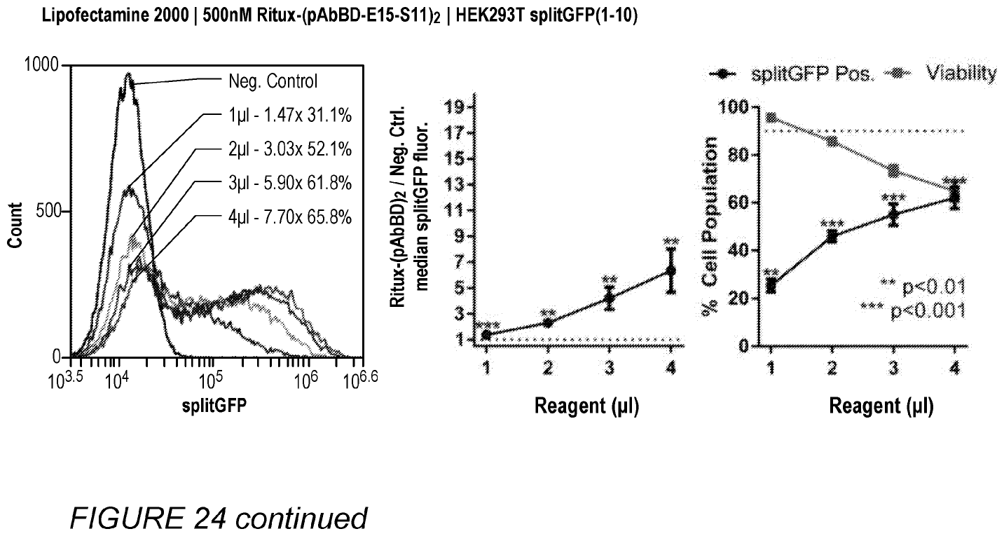

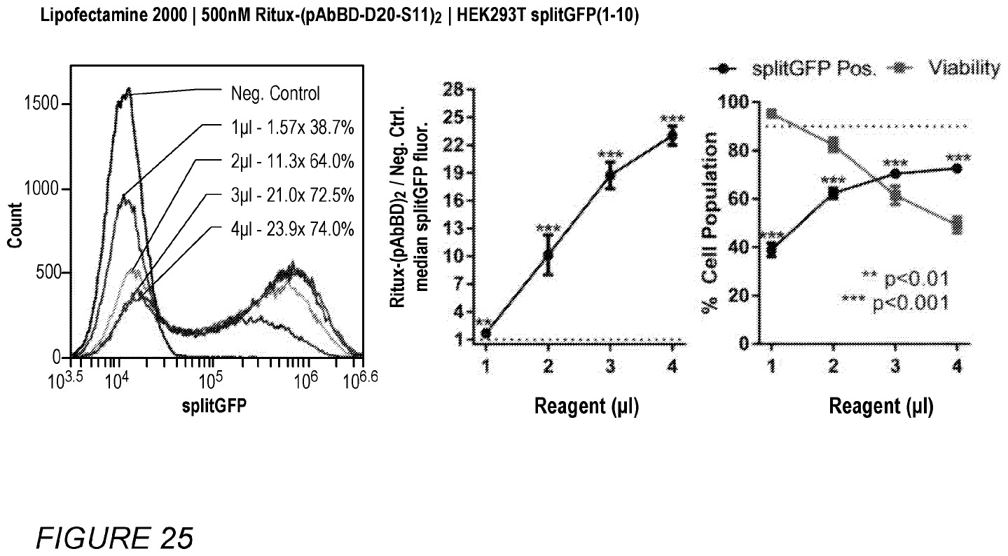

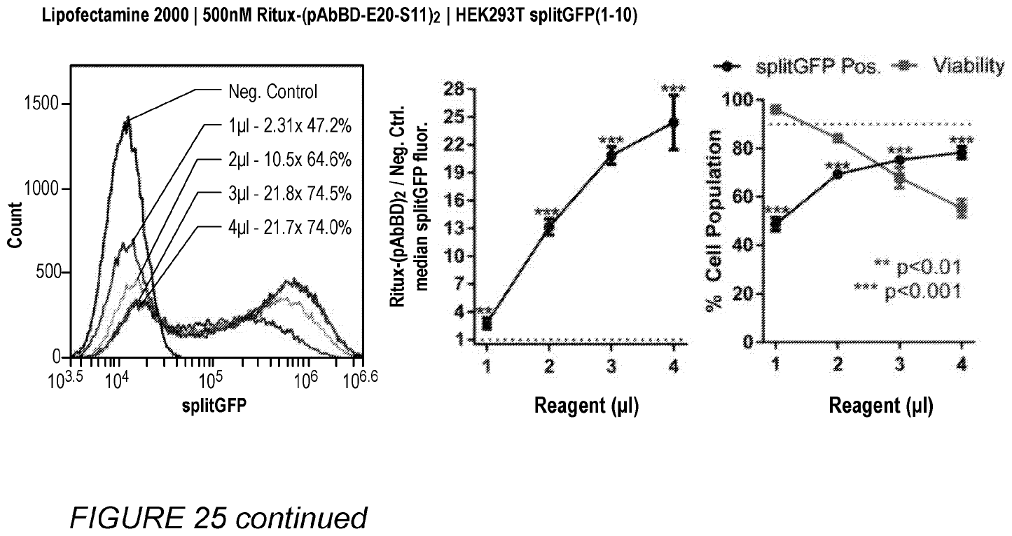

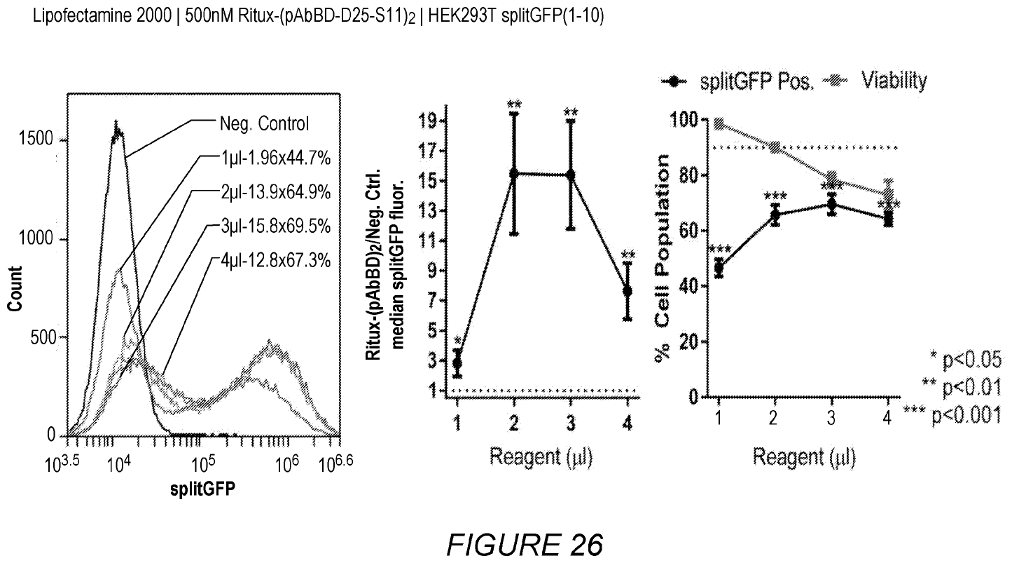

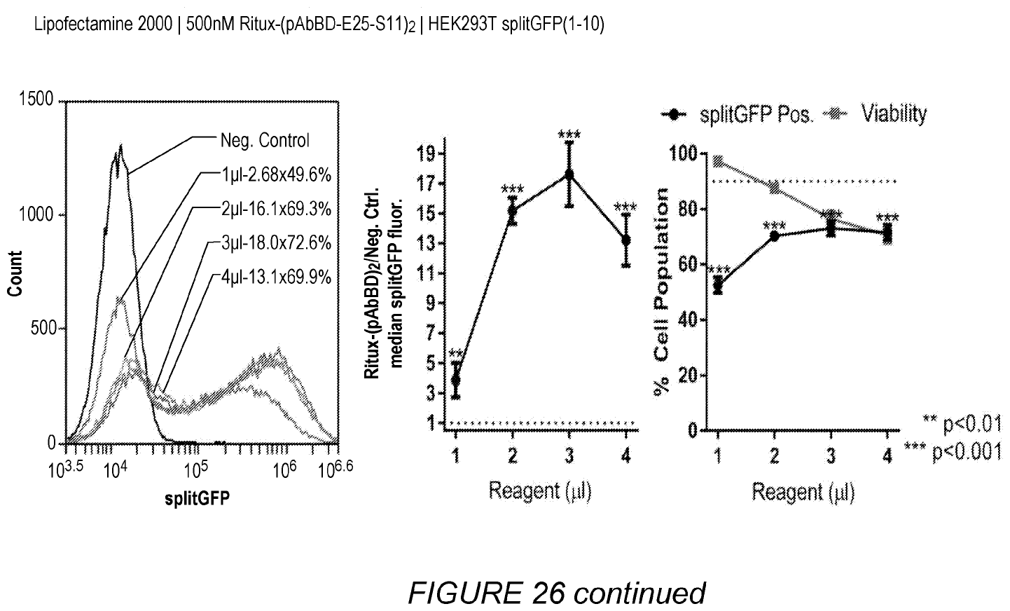

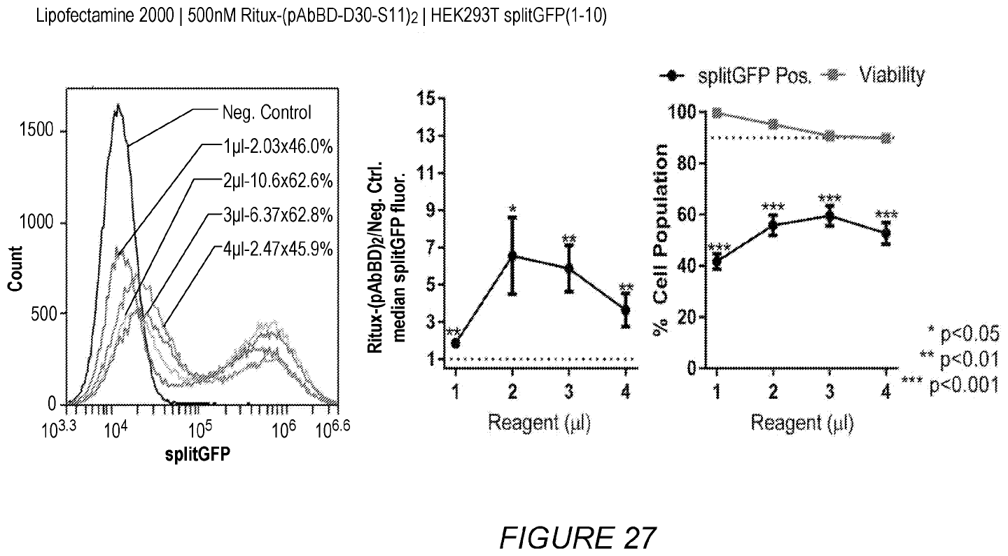

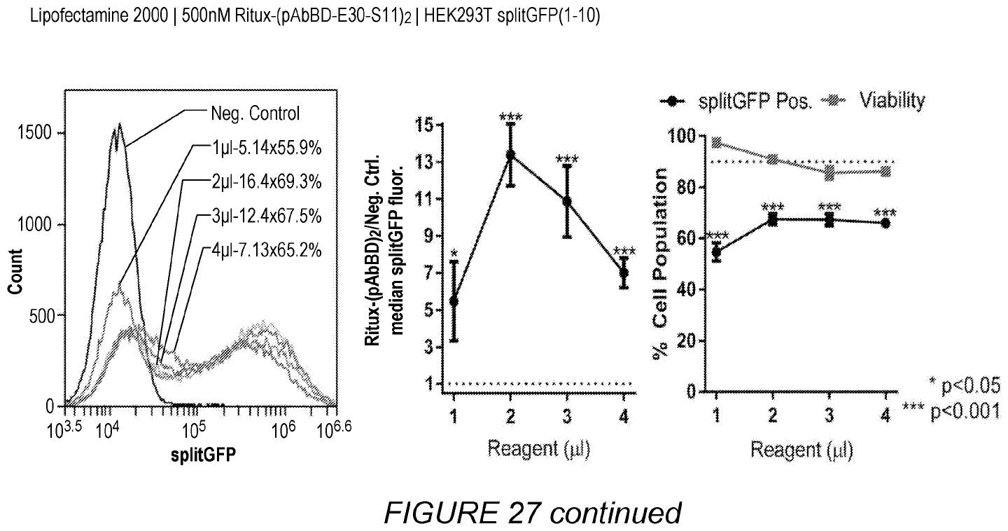

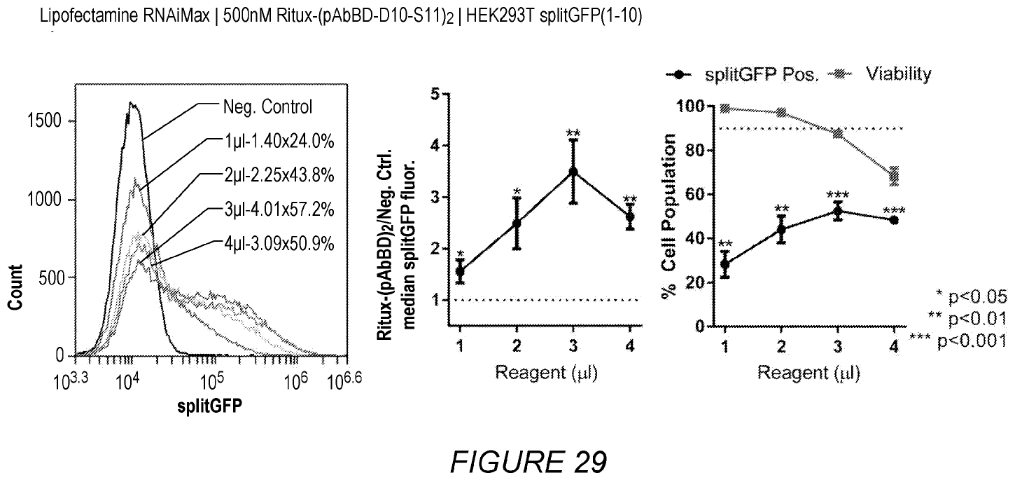

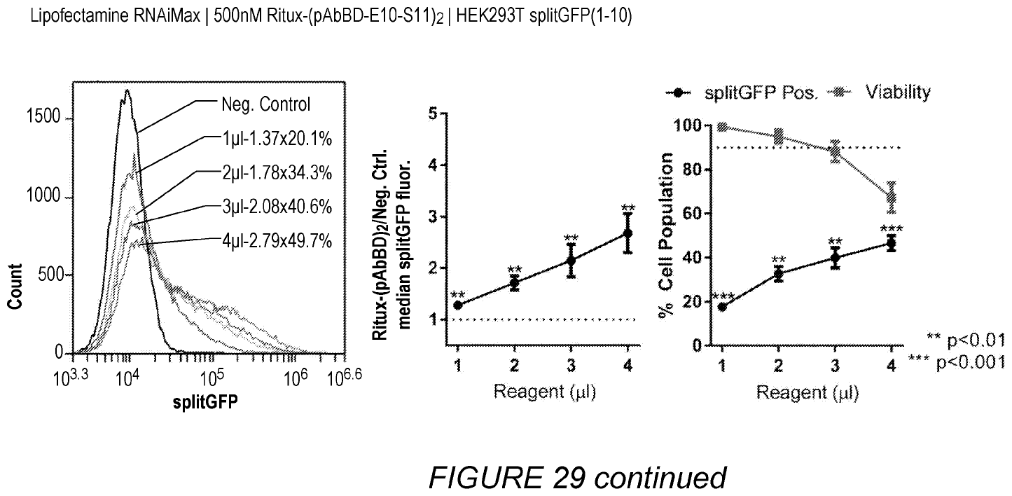

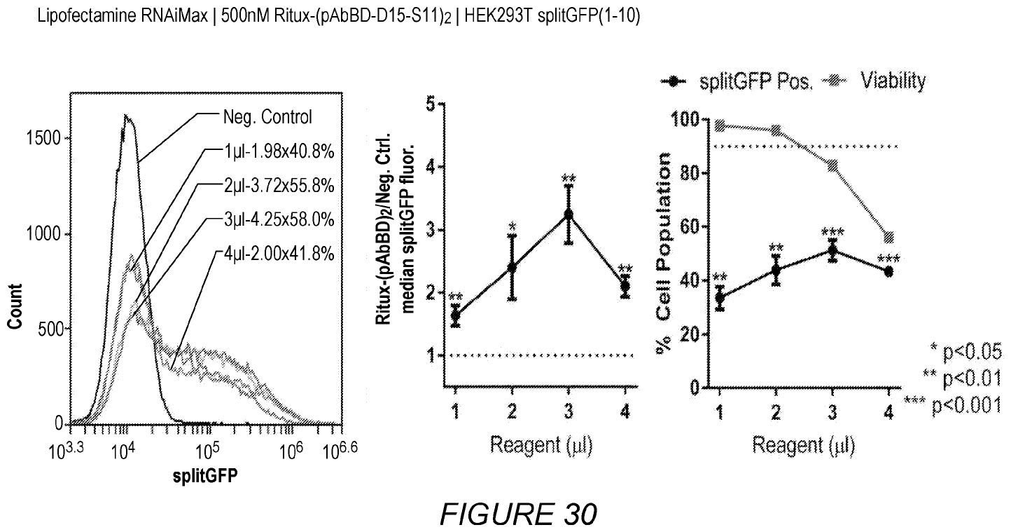

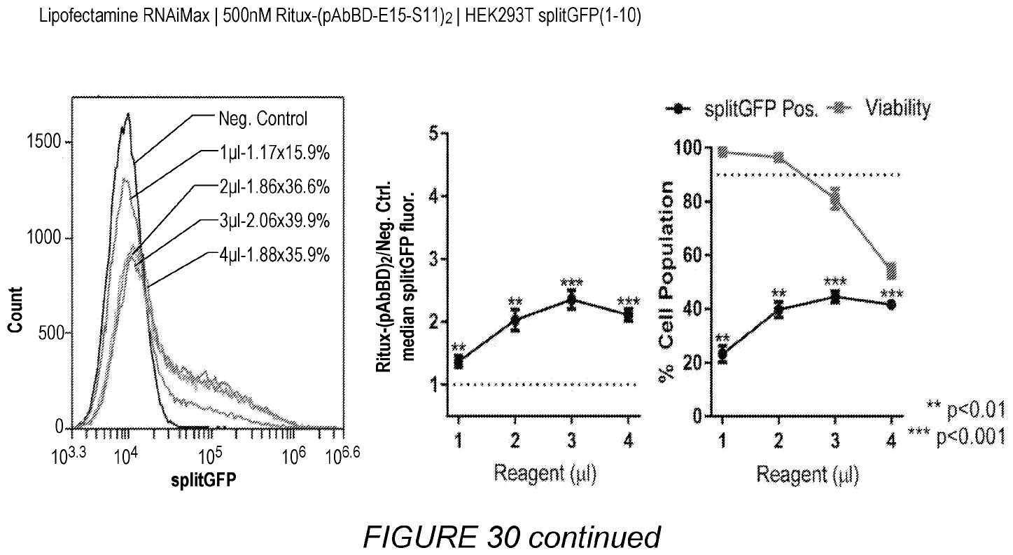

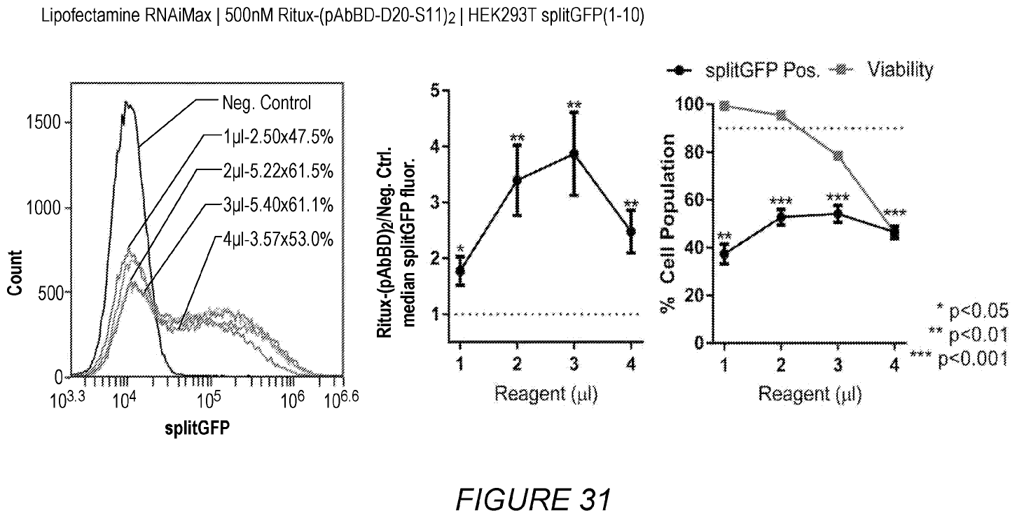

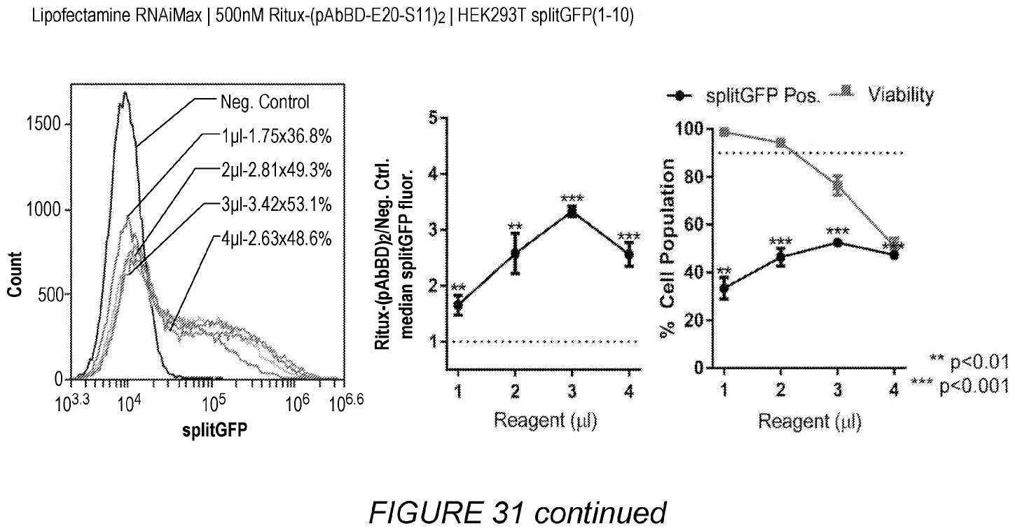

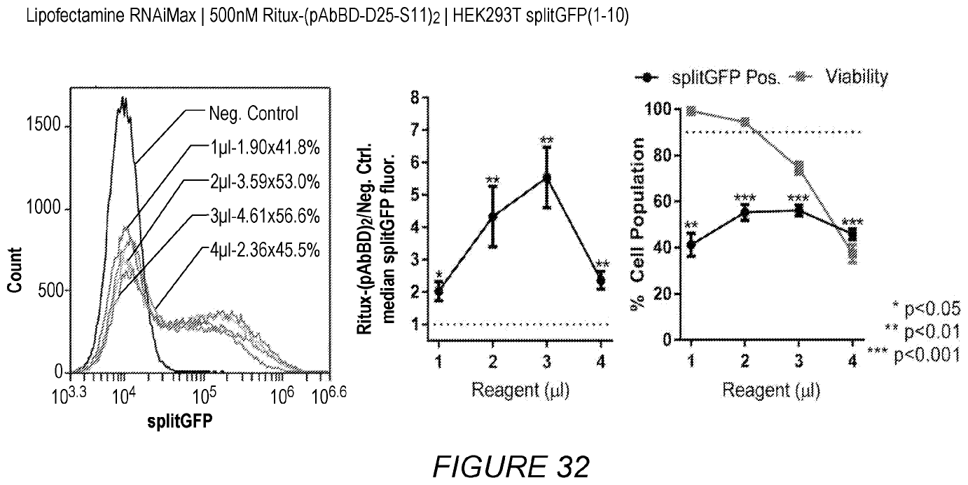

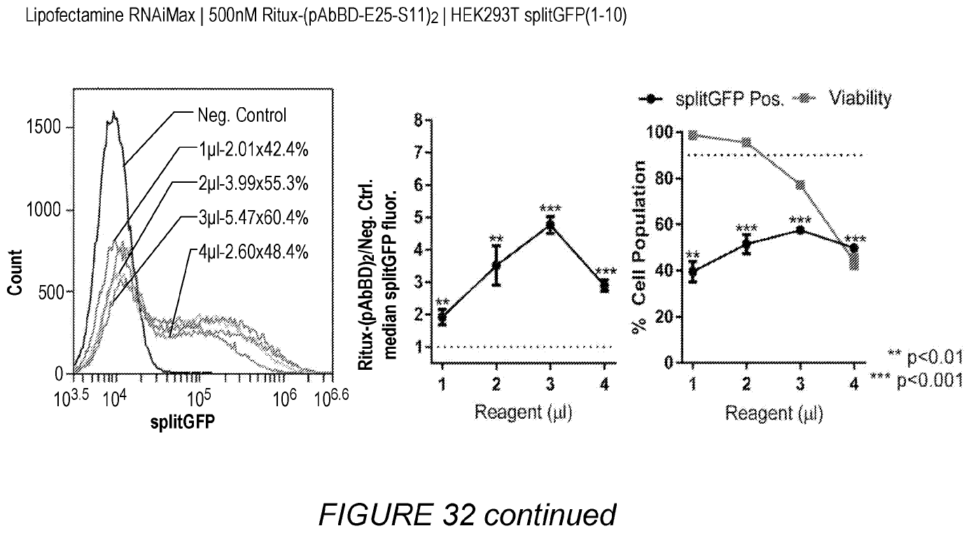

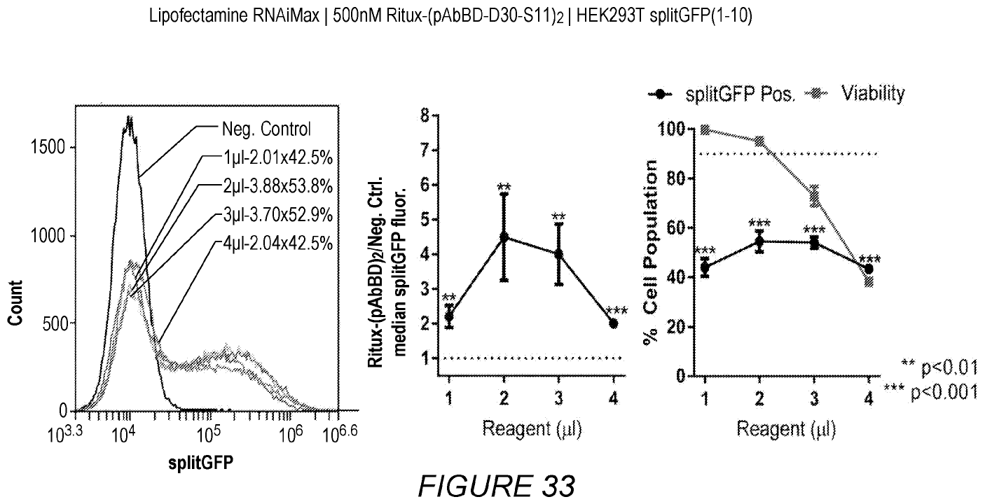

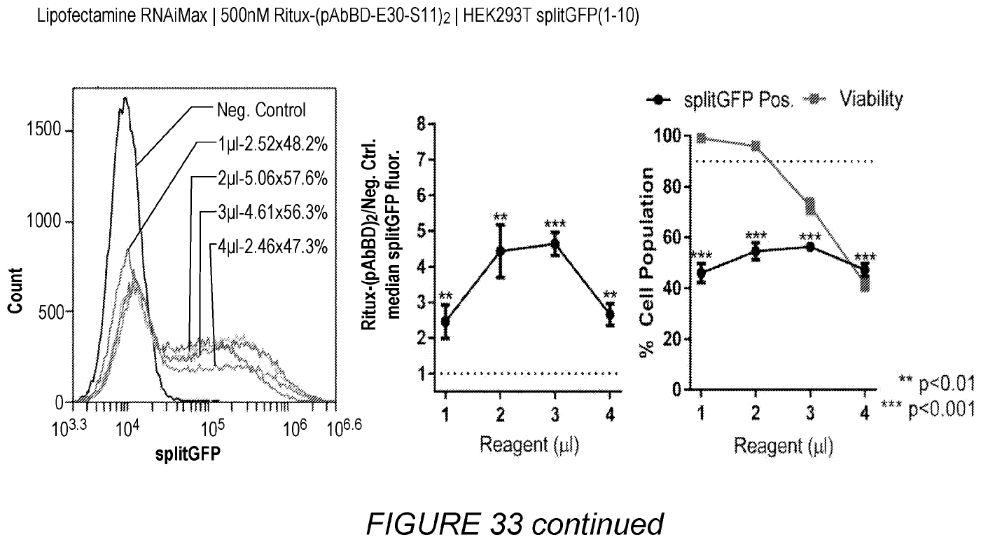

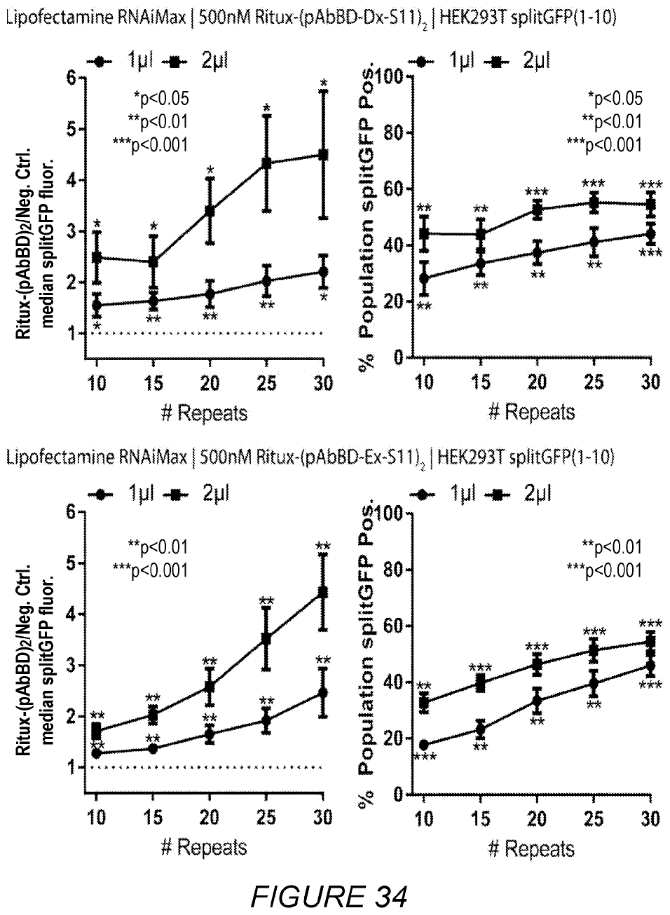

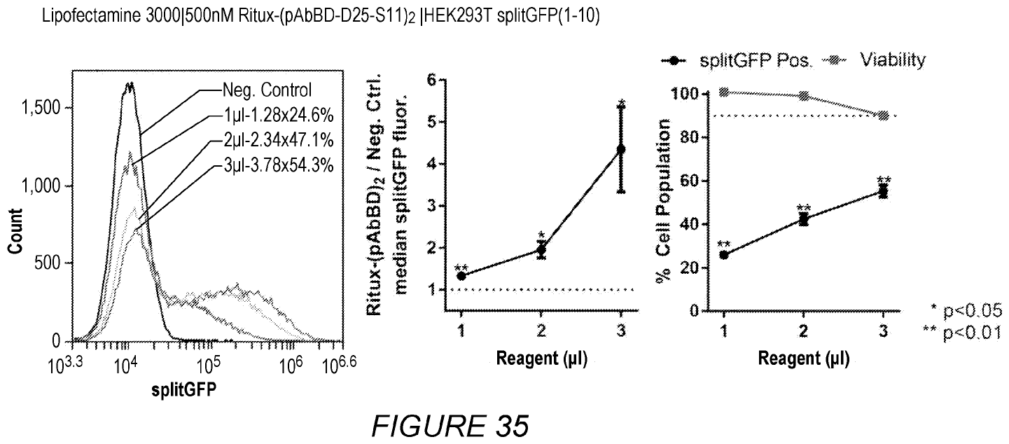

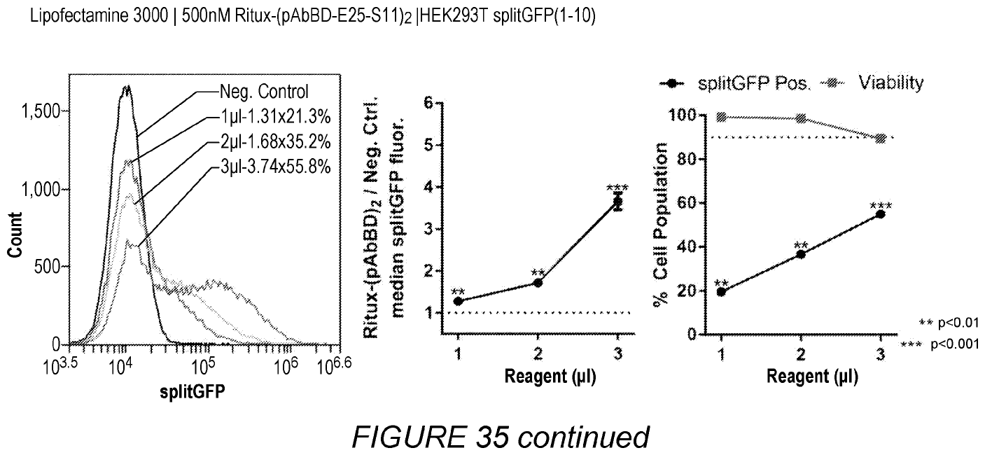

It was discovered that IgGs that are labeled with highly anionic polypeptides (ApPs) can be complexed with a variety of commercially available cationic lipids that were originally designed for gene delivery (e.g., Lipofectamine2000, Lipofectamine3000, RNAiMax) (FIGS. 19-35). These complexes can then be used to efficiently deliver the IgG into the cytoplasm of living cells. Nearly any IgG can be site-specifically and efficiently labeled with ApPs using pAbBD-ApP fusion proteins. The pAbBD-ApP is fused with the spitGFP S11 peptide to enable cytoplasmic delivery of the IgG-ApP conjugates to be monitored in cells engineered to express splitGFP(1-10). Upon successful cytosolic delivery, splitGFP complementation occurs between the splitGFP S11 peptide and the splitGFP(1-10), resulting in turn-on splitGFP fluorescence. No splitGFP fluorescence is observed if IgG conjugates with the S11 peptide are extracellular or within endosomal/lysosomal compartments.

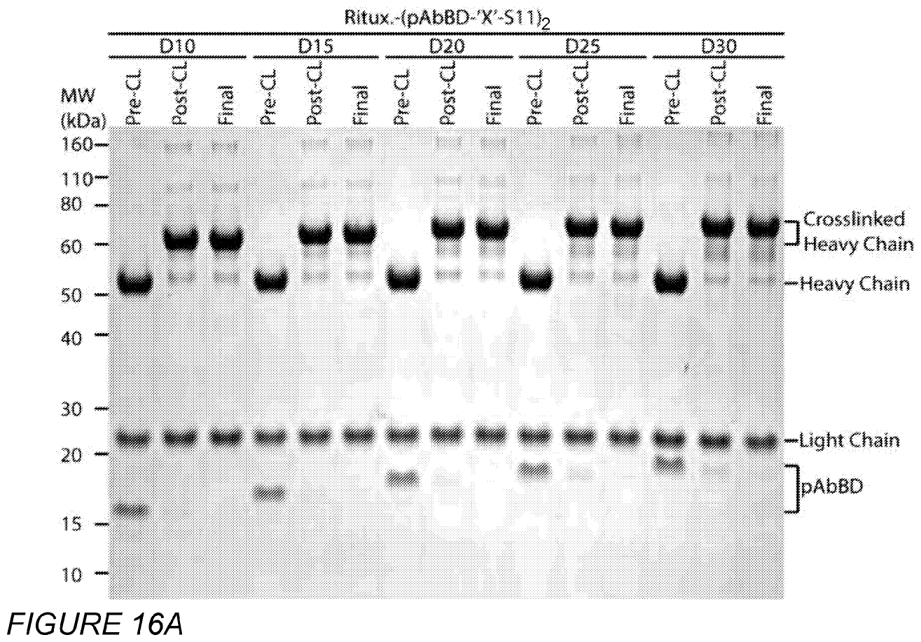

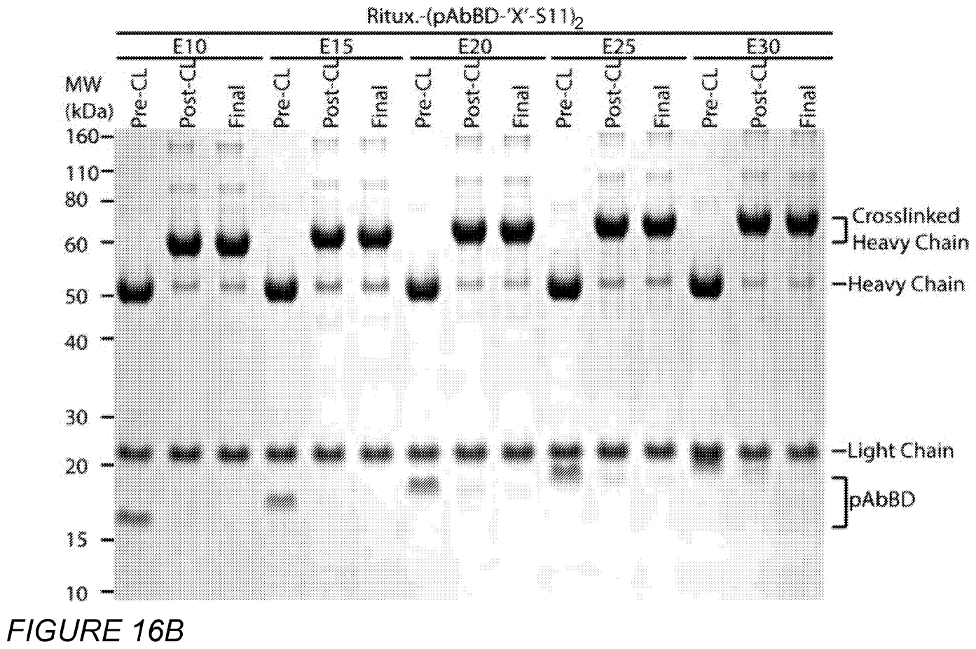

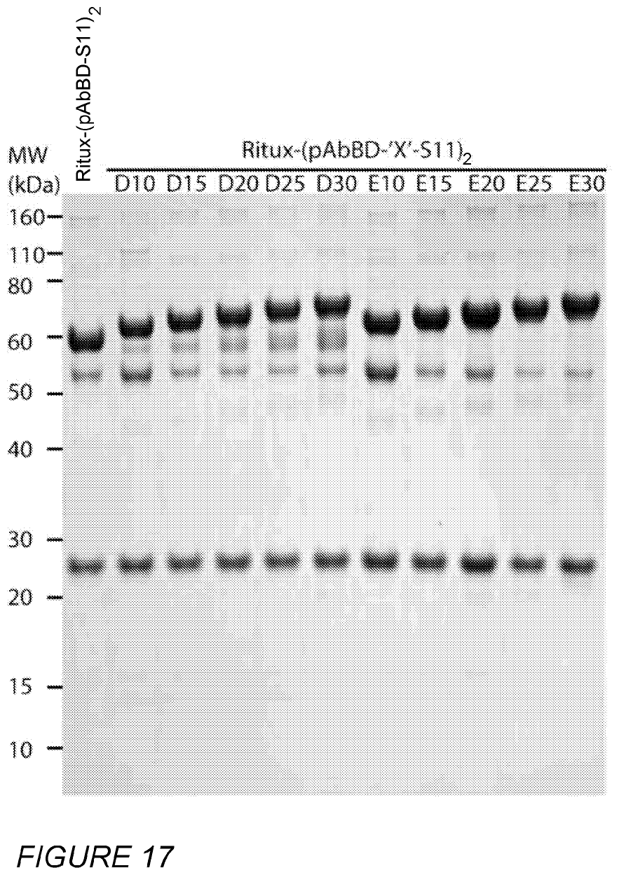

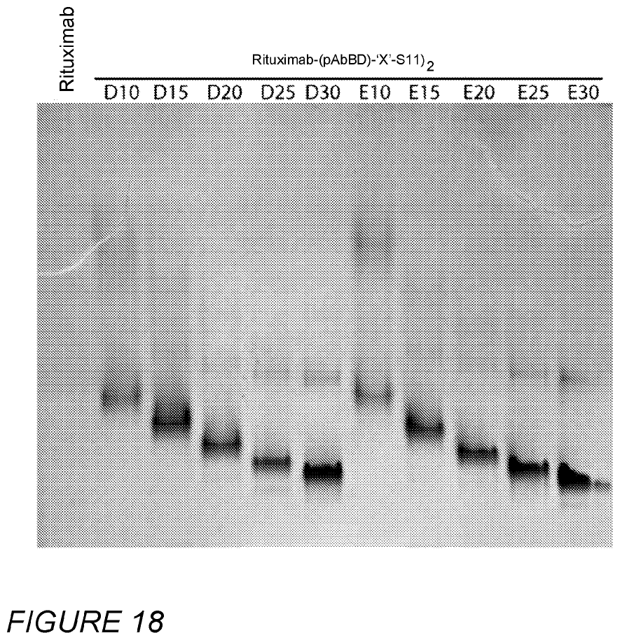

Preparation of anionic polypeptide-IgG conjugates (IgG-ApPs): This approach to antibody delivery requires IgG to be complexed with cationic transfection agents. This is accomplished through the attachment of ApPs composed of long repeats of aspartic acid (D), glutamic acid (E) or combinations thereof. The coding sequences for the ApPs are cloned downstream of the pAbBD, so they can be easily and site-specifically conjugated to any IgG of choice. The pAbBD-ApPs fusion proteins were prepared with 0, 10, 15, 20, 25, and 30 aspartic acid or glutamic acid repeats. In addition, the splitGFP S11 peptide was fused downstream of the ApPs to allow successful cytoplasmic antibody delivery to be easily detected by turn-on splitGFP fluorescence, in cells engineered to express the complementary splitGFP(1-10). To prepare the antibody conjugates, the pAbBD-ApP-S11 fusion proteins were simply mixed with the desired IgG and photocrosslinked for 4 hrs using non-damaging far-UV light (365 nm). An anti-CD20 antibody (Rituximab) was used to validate this approach, unless noted otherwise, since it is not expected to bind to any intracellular or extracellular targets in the engineered cell lines. This allows us to purely study cytoplasmic delivery, without any complicating factors that could be associated with binding. Evaluation of IgG-ApP conjugates by SDS-PAGE (FIG. 16-18) confirmed that >95% of the heavy chains were covalently linked to the pAbBD-ApP-S11 fusion proteins, i.e. two pAbBD-ApP-S11 per IgG (one per heavy chain). Free pAbBD-ApP is easily removed by filtration (100 kDa MWCO, Millipore), due to the low molecular weight of the pAbBD-ApP-S11 fusion proteins compared with IgG.

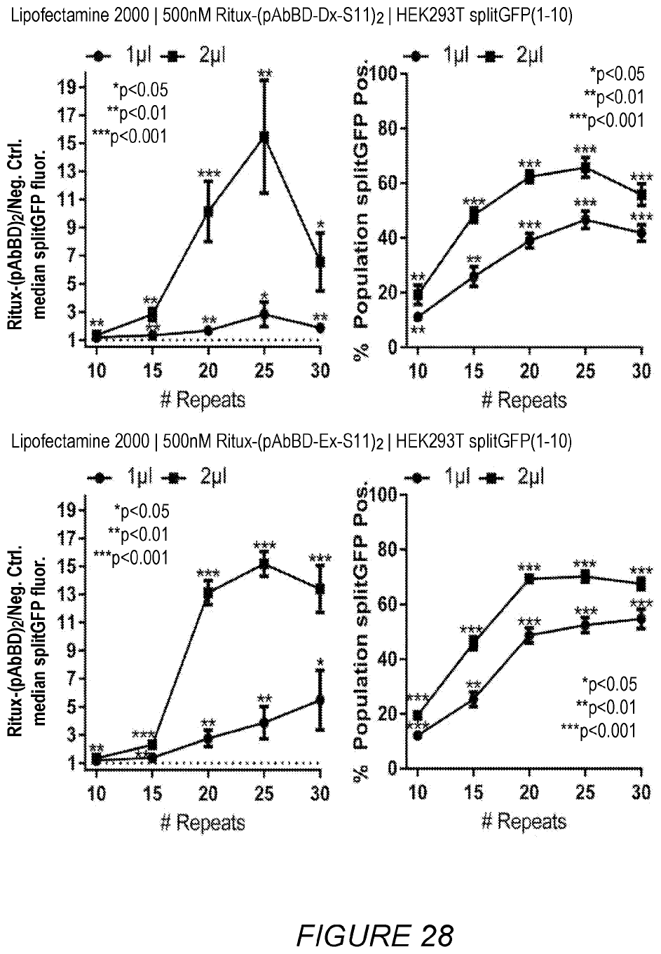

Cytoplasmic delivery of ING-ApP conjugates: Once IgG-ApPs were prepared, they were complexed with Lipofectamine 2000 according to the manufacturer's protocol. The complex was then added to HEK293T splitGFP(1-10) cells at a final antibody concentration of 500 nM for 6 hrs. The cells were then washed and analyzed by fluorescence microscopy or flow cytometry. Turn-on splitGFP fluorescence increased as the length of the ApP was increased up to 25 aspartic residues and then seemed to decrease with longer chain lengths (FIG. 28). When glutamic acid was used, turn-on splitGFP fluorescence increased as the length of the ApP was increased up to 25 residues (FIG. 28). The splitGFP fluorescence was generally confined to the cytosol (FIGS. 19-22), with the nucleus appearing darker, presumably due to the inability of the large IgG to passively cross nuclear pore complexes. This was not evident when the pAbBD-ApP-S11 fusion proteins (without IgG) were delivered into cells. The much smaller fusion proteins were able to diffuse into the nucleus and turn-on splitGFP fluorescence was evident throughout the cell, including the nucleus.

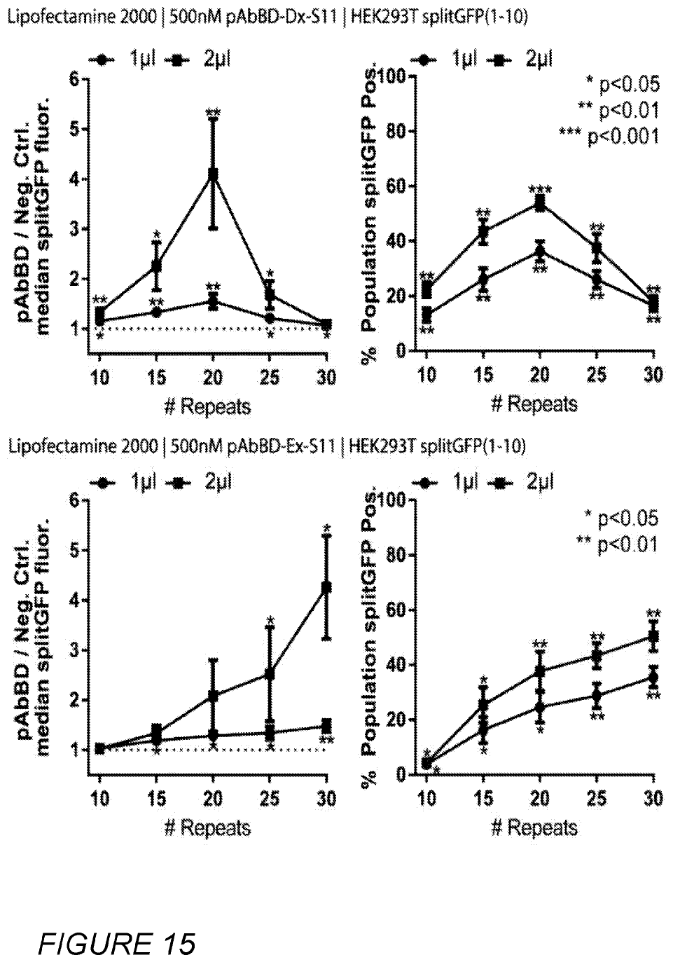

When ApPs had >20 anionic amino acid residues, >60% of the cells were found to be positive for splitGFP fluorescence with a median fluorescence that could be more than 15 times higher than the negative cell population (FIG. 28), which consist of cells that undergo the same procedure but with IgG conjugated to pAbBD-S11 fusion proteins without ApPs. Technical and biological quadruplicates were acquired for all studies. Interestingly, the transfection efficiency was marginally lower with the pAbBD-ApP-S11 fusion proteins (without IgG), with up to ˜50-55% of cells being positive for splitGFP fluorescence and a ˜4-fold increase in median fluorescence (FIG. 15).

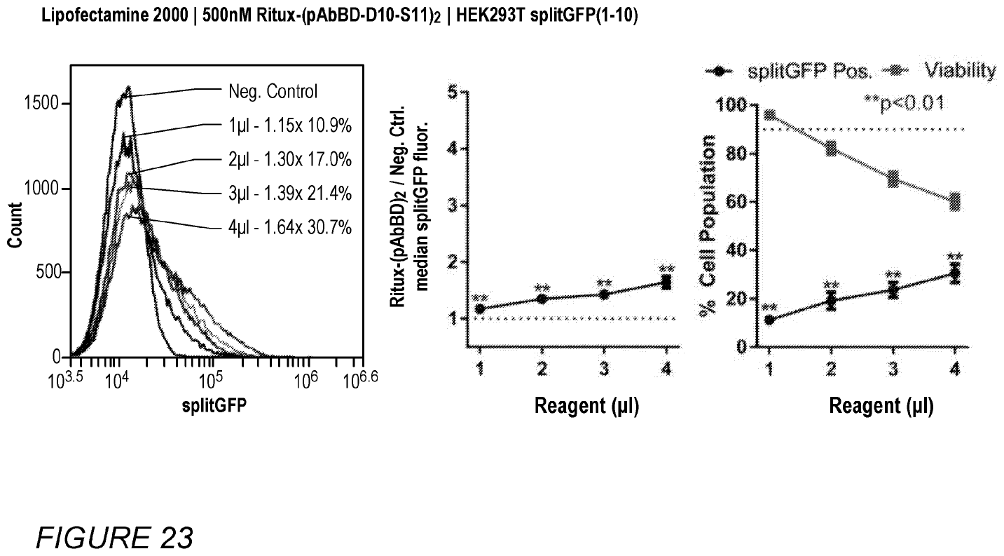

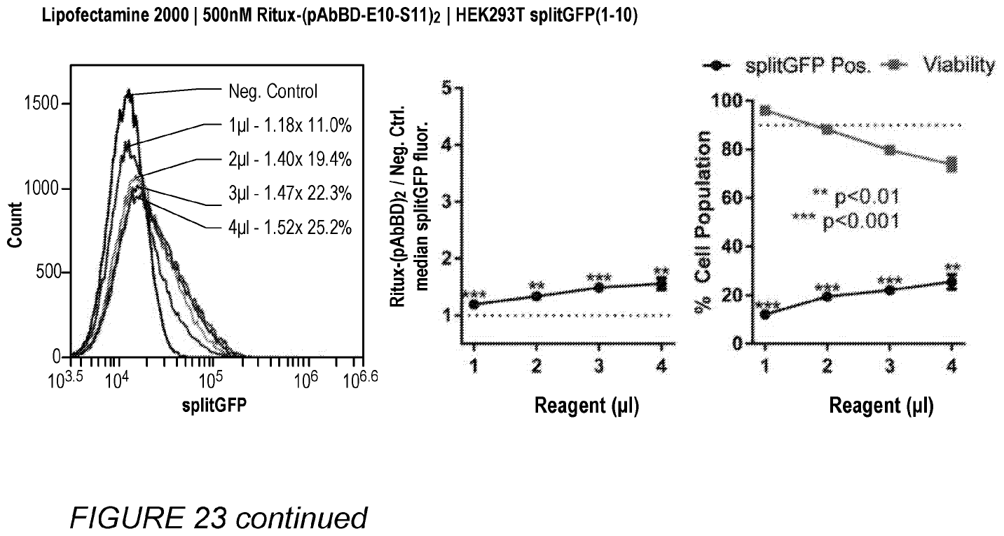

Similar to when Lipofectamine 2000 is used for gene delivery, transfection efficiency and cell viability are both dependent on the amount of the Lipofectamine 2000 reagent that is utilized (FIG. 23-27). At a fixed concentration of IgG-ApP (500 nM), it was found that as the amount of Lipofectamine 2000 reagent was increased from 1 to 2 μL, the median splitGFP fluorescence in HEK293T splitGFP(1-10) cells more than doubled; however, a further increase in Lipofectamine 2000 did not always lead to an improvement in median fluorescence and sometimes decreased. The percent of the cell population that was positive for splitGFP fluorescence generally increased with the amount of Lipofectamine 2000, but 2 μL of the reagent was still close to the optimum with a transfection efficiency of ˜65% with ApP lengths of 20 more anionic residues. In general, cell viability was inversely correlated with concentration of Lipofectamine 2000; however, at 2 μL the cell viability was still >90% in many cases.

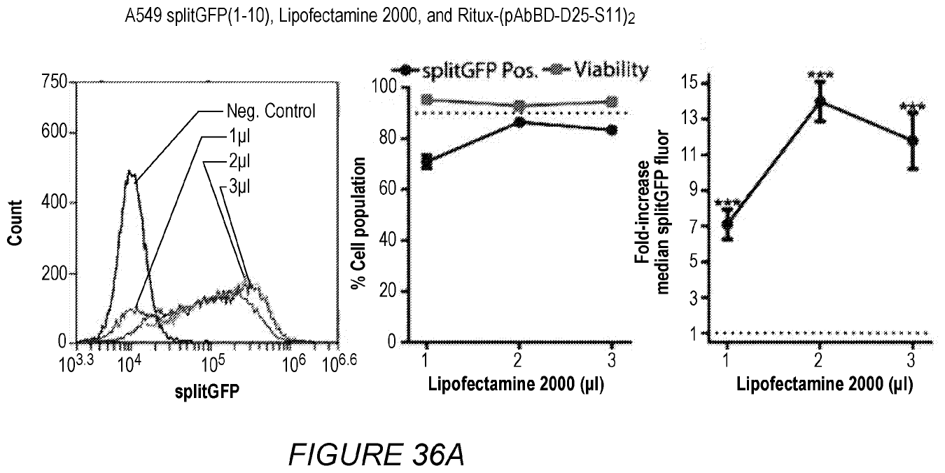

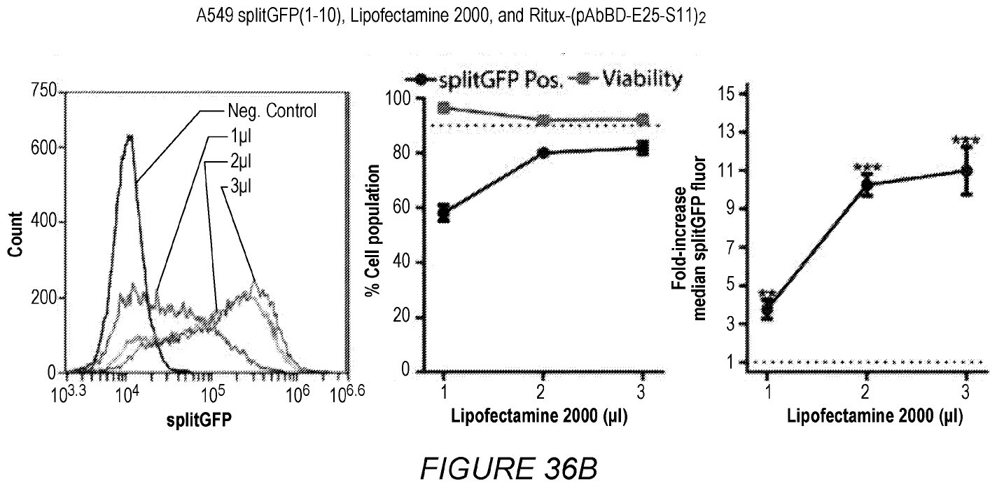

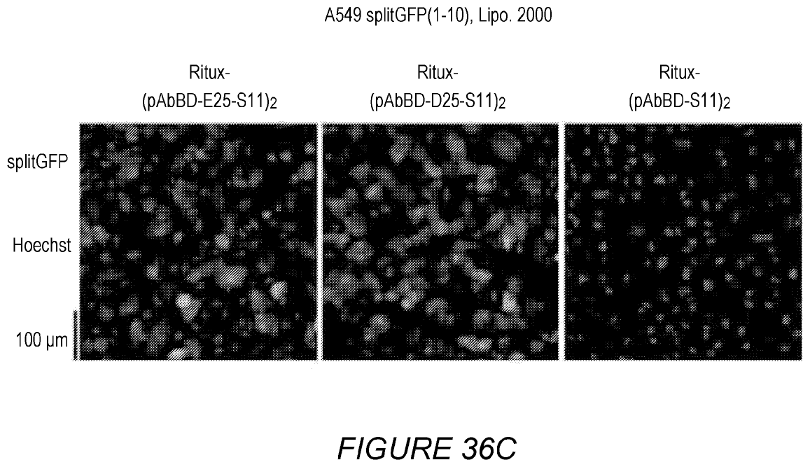

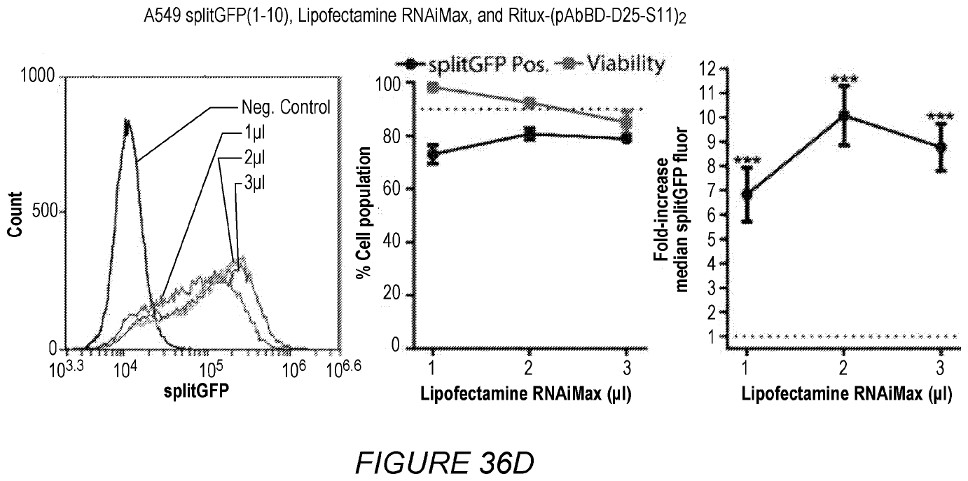

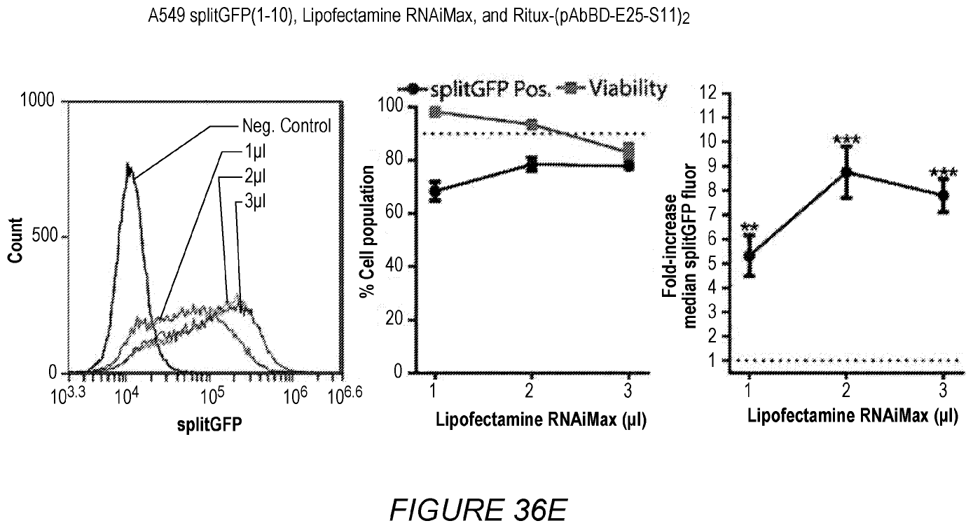

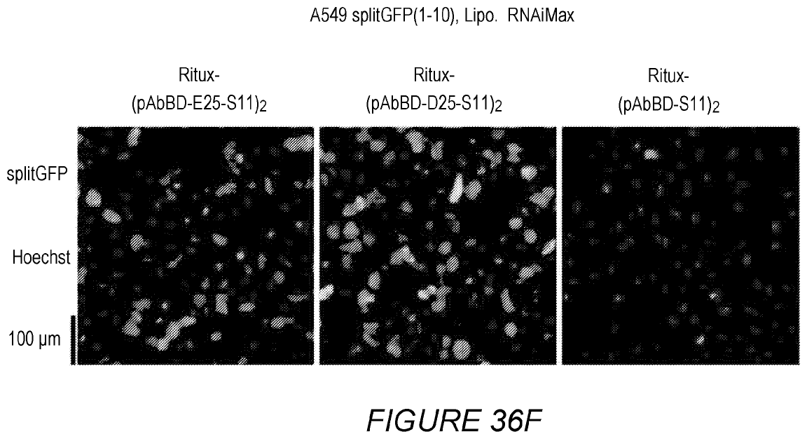

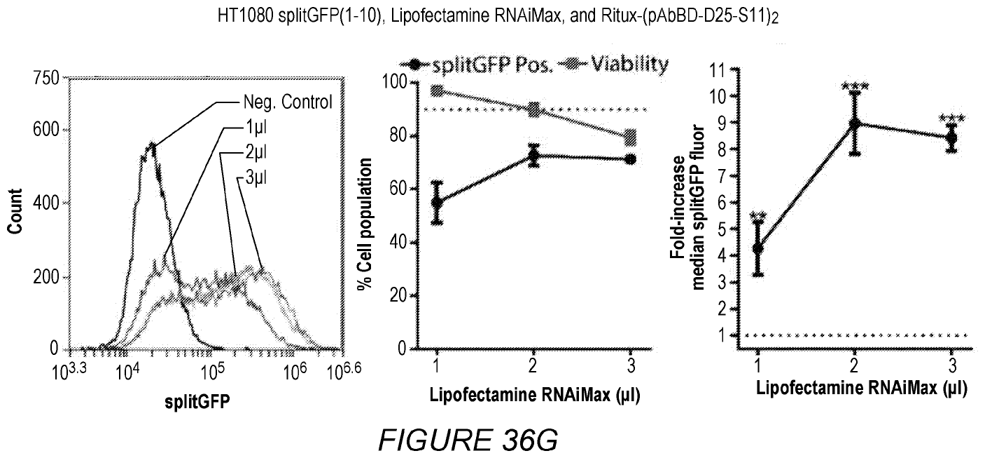

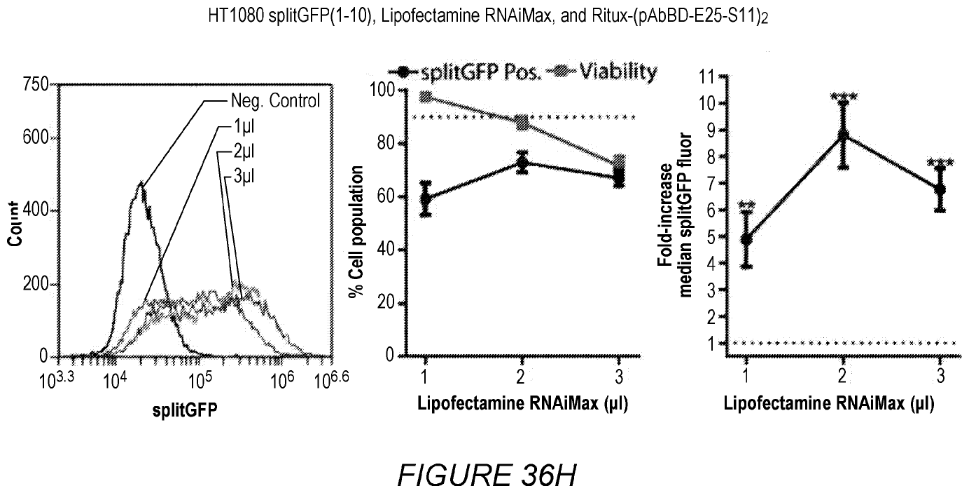

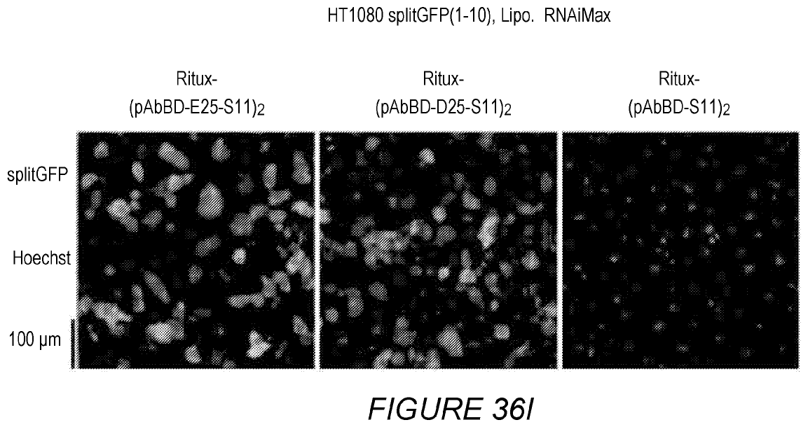

In addition to delivering antibodies into the cytoplasm of HEK293T splitGFP(1-10) cells, it was also demonstrated that cationic lipids could be used to deliver IgG-ApP conjugates into the cytoplasm of A549-splitGFP(1-10) cells and HT1080-splitGFP(1-10) cells (FIG. 36). Greater than 50% transfection efficiency was achieved in both cell types, pointing to the generalizability of this approach.

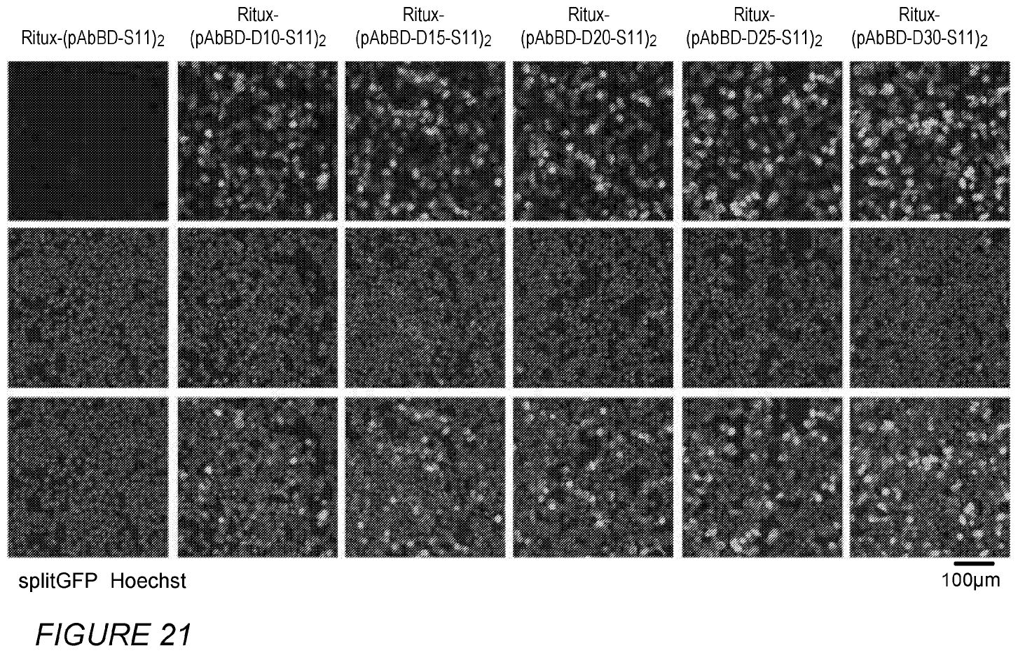

Antibody delivery was not limited to the use of Lipofectamine 2000, but was also achieved with RNAiMax and Lipofectamine 3000 (FIG. 21, 22, 29-35). RNAiMax and Lipofectamine 3000 proved to be slightly less efficient with the conditions tested. Nonetheless, these results again highlight the generalizability of this approach and show that cationic lipid formulations that are more appropriate for systemic delivery can also be utilized for cytoplasmic antibody delivery.

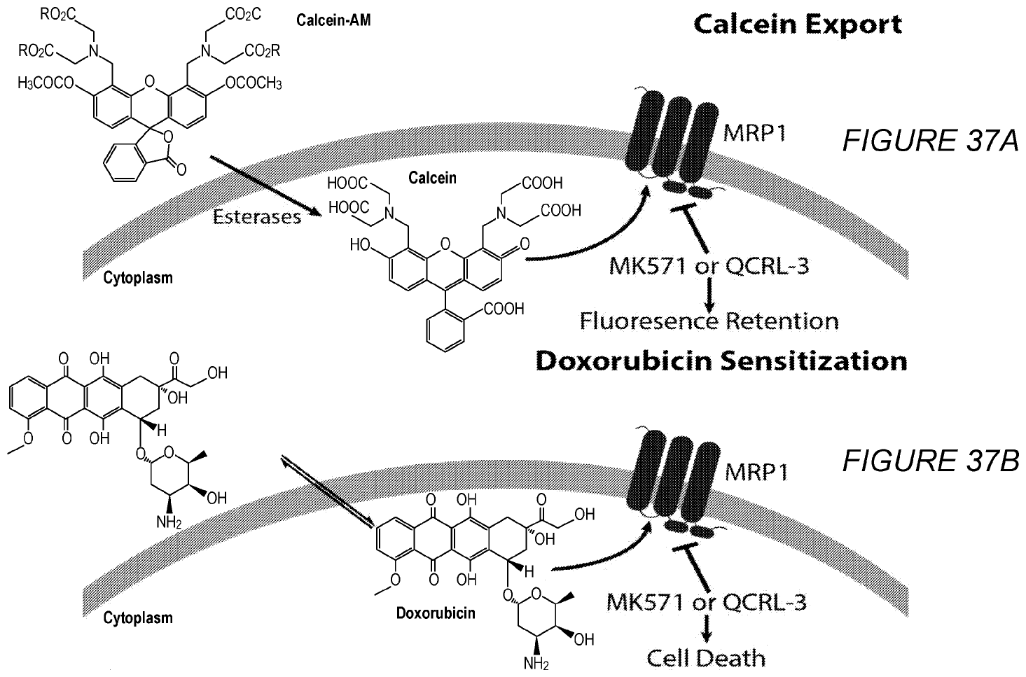

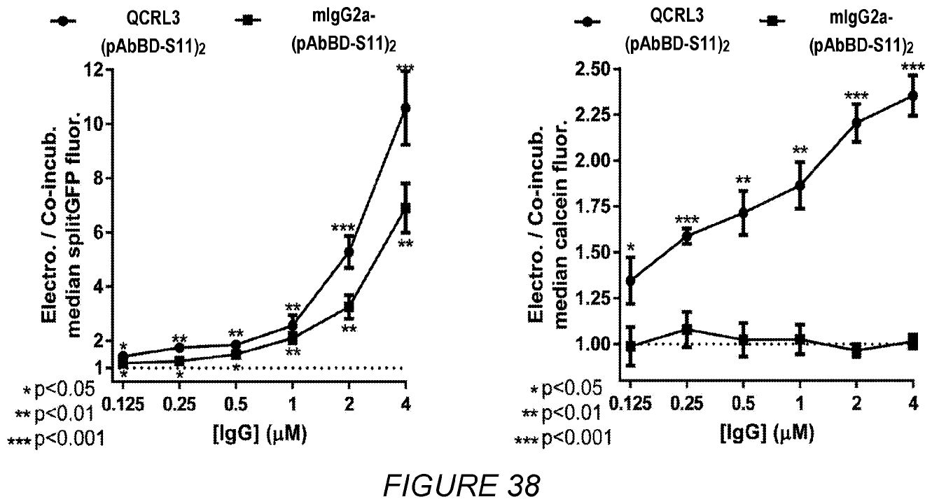

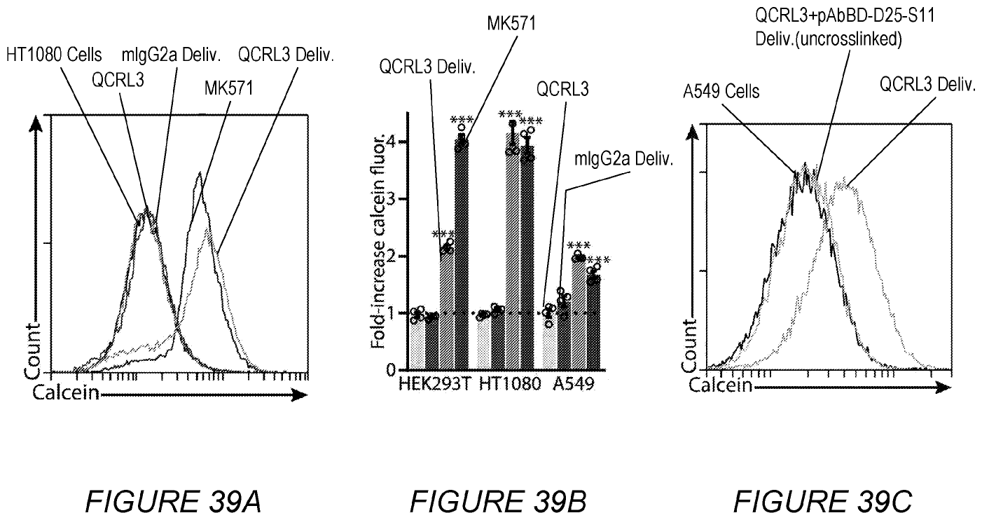

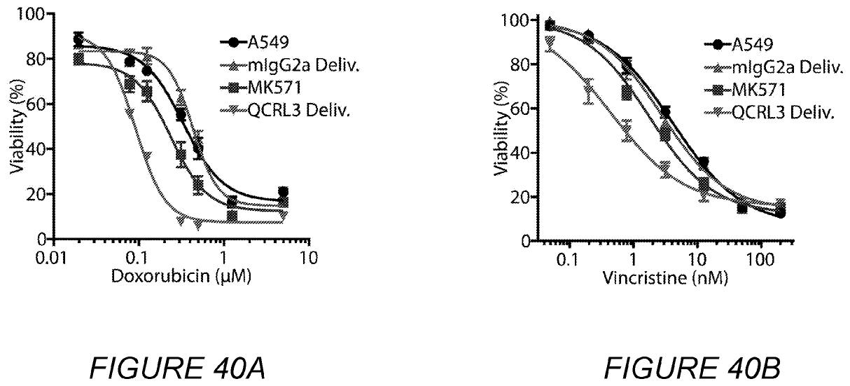

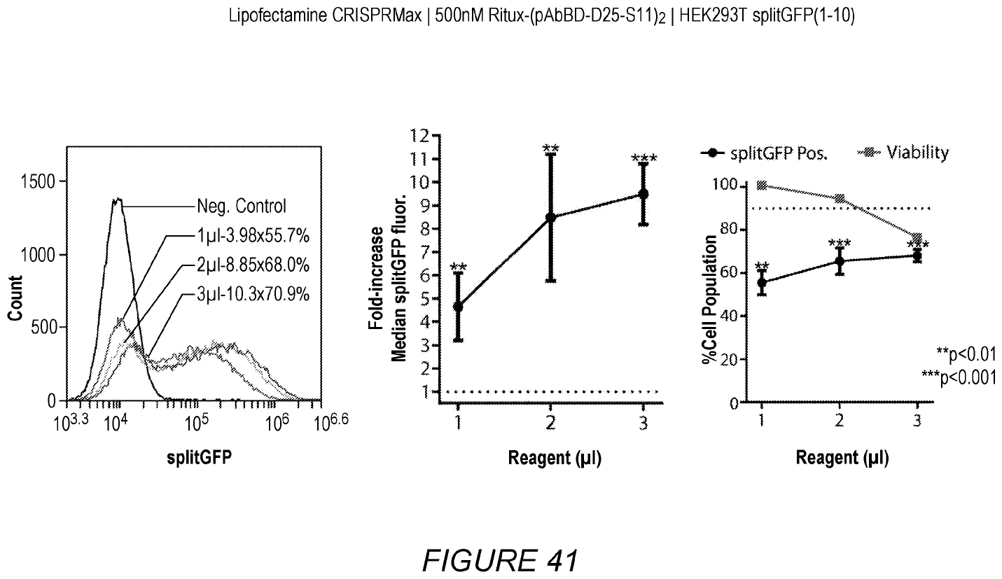

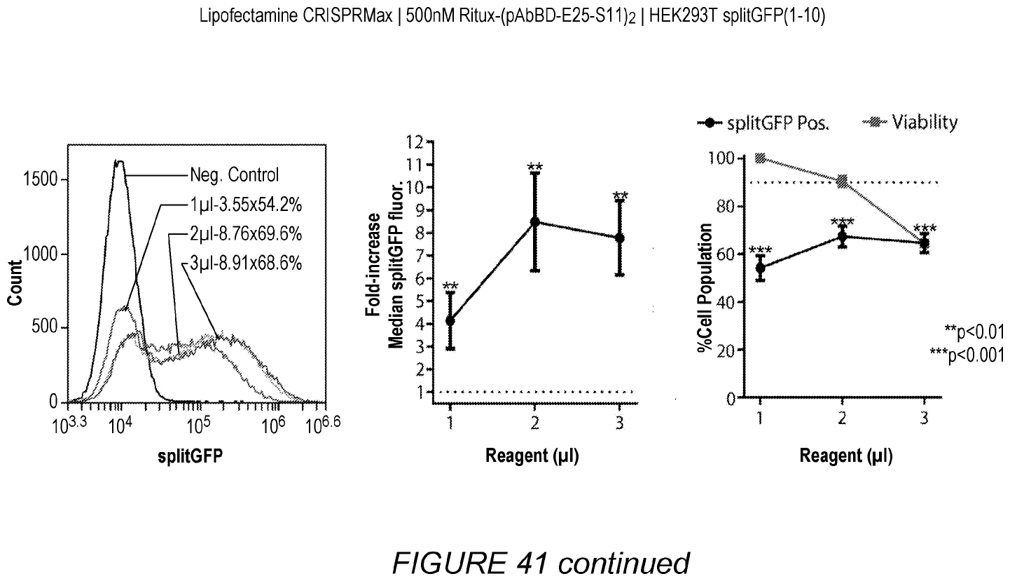

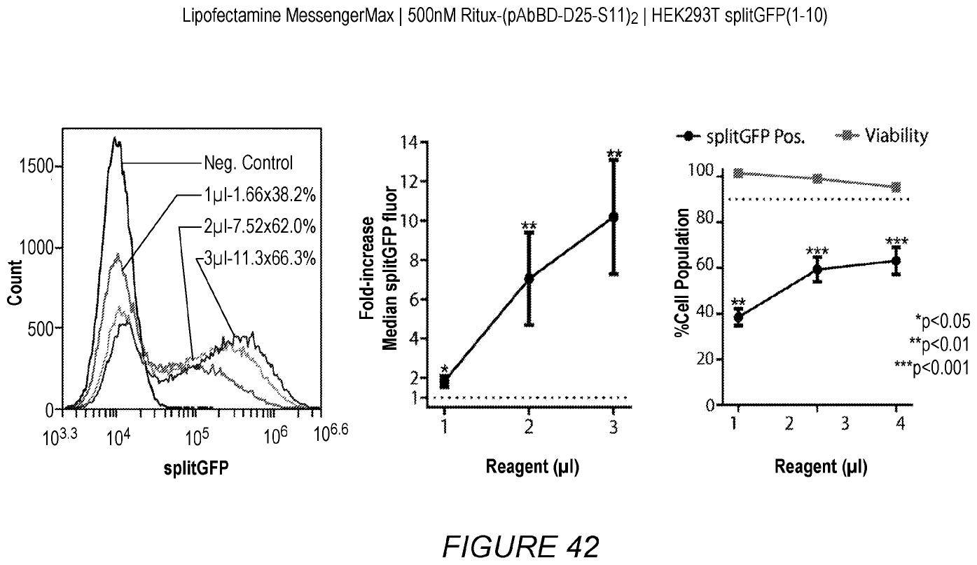

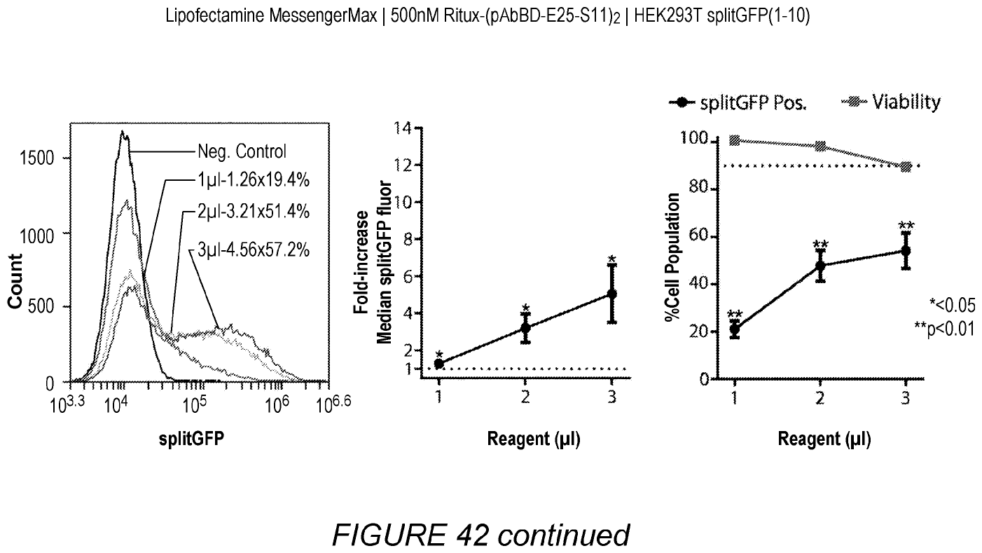

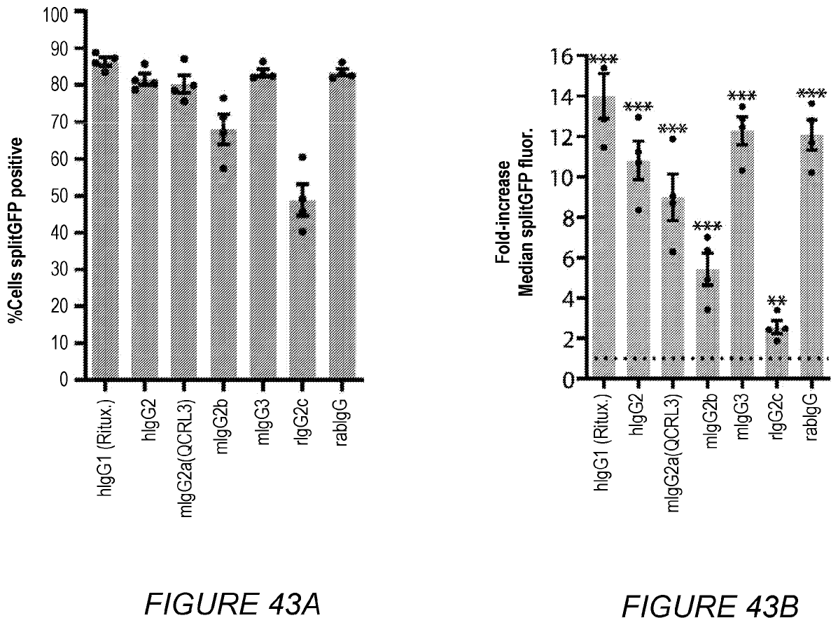

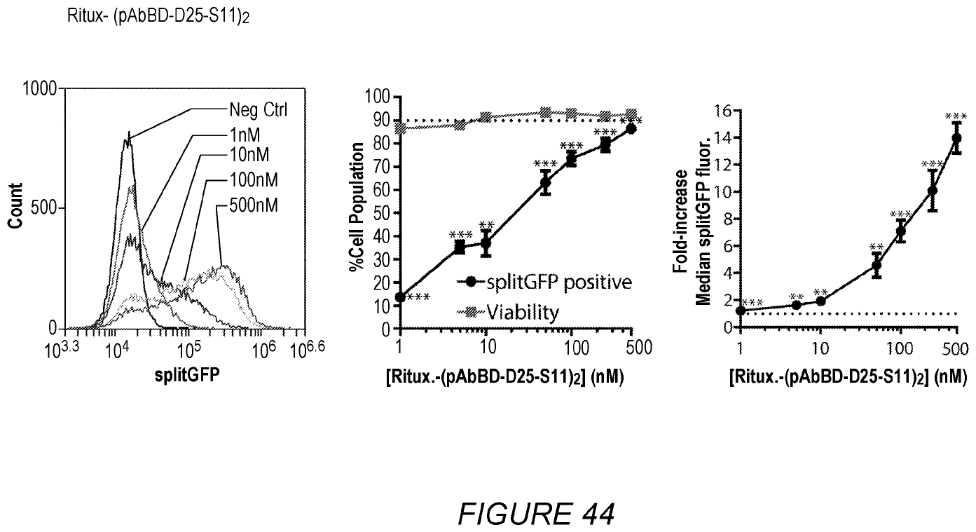

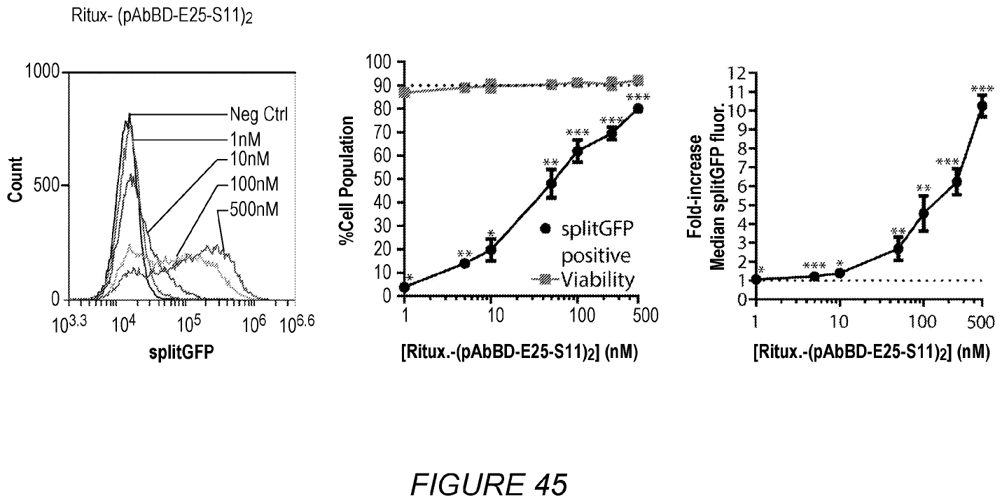

Antibody-mediated inhibition of MRP1: To demonstrate that antibodies in the cytoplasm are functionally active and not inactivated by the reducing intracellular environment, a calcein export assay was performed (FIG. 37A). In this assay, cells were first incubated with calcein-AM, a non-fluorescent membrane permeable calcein analog. Intracellular esterases cleave calcein-AM to calcein, which is not only fluorescent, but also accumulates intracellularly since it is membrane impermeable. Cells with high MRP1 activity will rapidly export calcein yielding cells with low fluorescence; however, cytoplasmic delivery of the anti-MRP1 antibody will inhibit calcein export and will result in higher fluorescence due to calcein retention. QCRL3, an anti-MRP1 antibody was used that has previously been shown to inhibit MRP1 activity. QCRL3 (500 nM) was cytoplasmically delivered into HEK293T cells with Lipopectamine 2000. The cells were then incubated with calcein-AM for 30 minutes and then allowed to export calcein for 16 h. Cellular fluorescence was analyzed by flow cytometry at this time (FIG. 39). For comparison, analogous studies were performed with an isotype matched antibody (mIgG2a). The negative control consisted of cells that were not treated with IgG-lipid complexes. It was found that intracellular QCRL3-ApP-S11 conjugates were able to inhibit calcein export, leading to a statistically significant increase in median cellular fluorescence compared to cells treated with mIgG2a. This suggests that cytoplasmically delivered antibodies are functionally active and can be delivered in sufficient quantities to inhibit normal cellular activity.

### Example 2

### IgG Conjugates for Maximum Cytoplasmic Delivery

The efficiency of cytoplasmic delivery with IgG-ApP-S11 conjugates, with 10, 15, 20, 25, and 30 aspartic acid or glutamic acid residues, was tested using three different transfection agents and three different cell lines. While these findings provide strong evidence of cytoplasmic delivery, there are still many additional variables that can be explored. Most notably, one can explore the dependence of cytoplasmic delivery on incubation time and the concentration of IgG-ApP-S11. All studies were performed with 500 nM IgG (final concentration). In these studies, transfection efficiency was found to generally increase with the number of glutamic acid residues in the ApP. One can prepare additional ApPs with 35 or more residues to see if transfection efficiency can be increased further. One can also explore whether mixtures of glutamic acid and aspartic acid residues perform any better than homopolypeptides or if the placement of uncharged amino acids between the glutamic acid or aspartic acide improves cytoplasmic delivery. Finally, one can continue and try additional transfection agents to see if even higher delivery efficiencies can be achieved. Once optimized conditions for cytoplasmic antibody delivery have been realized, one can explore two distinct applications. In the first application, one can evaluate the ability of anti-MRP1 antibodies to inhibit calcein export and in a more biologically relevant assay, export of the chemotherapeutic doxorubicin (Dox). In particular, on can test whether antibody-mediated MRP1 inhibition can sensitize cells to Dox-triggered cell death. In a second demonstration of intracellular targeting, one can use an anti-Ras antibody to inhibit Ras-dependent signaling.

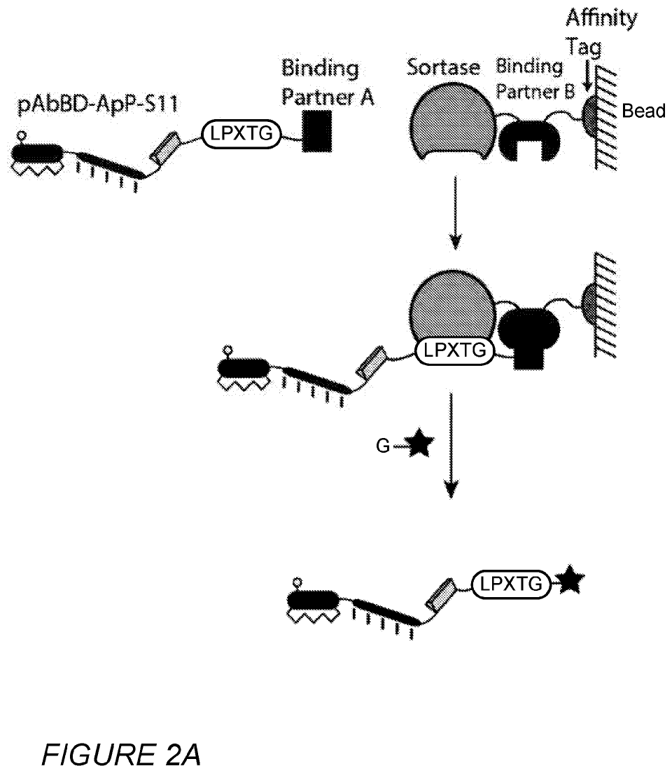

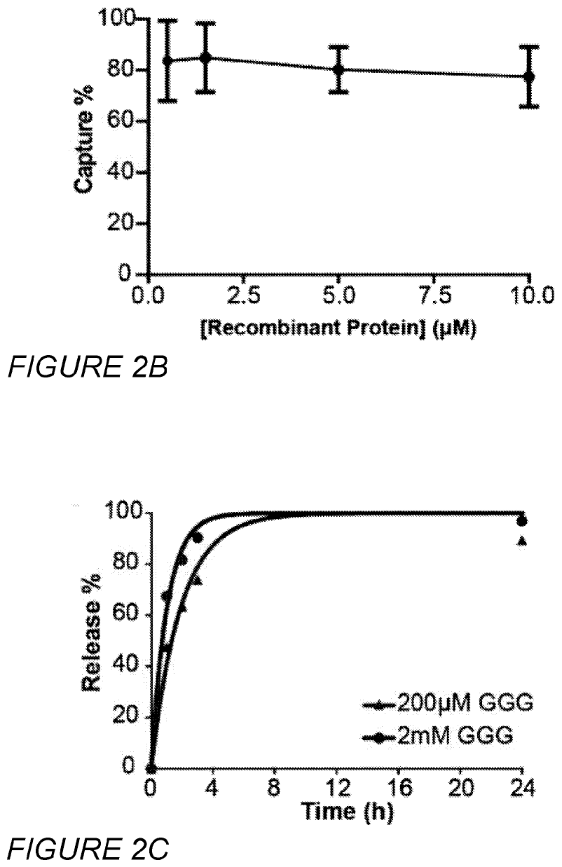

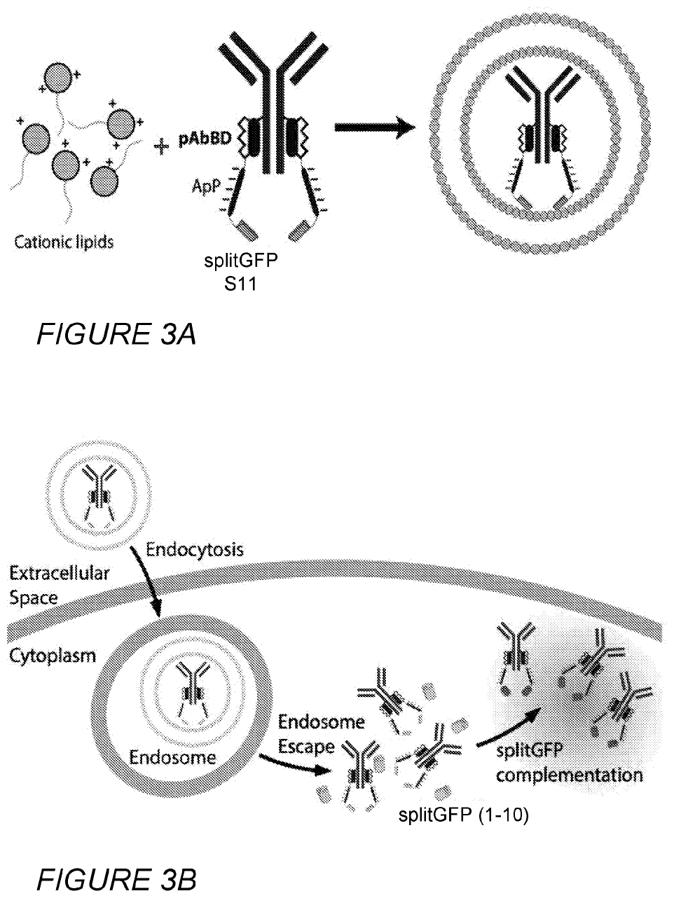

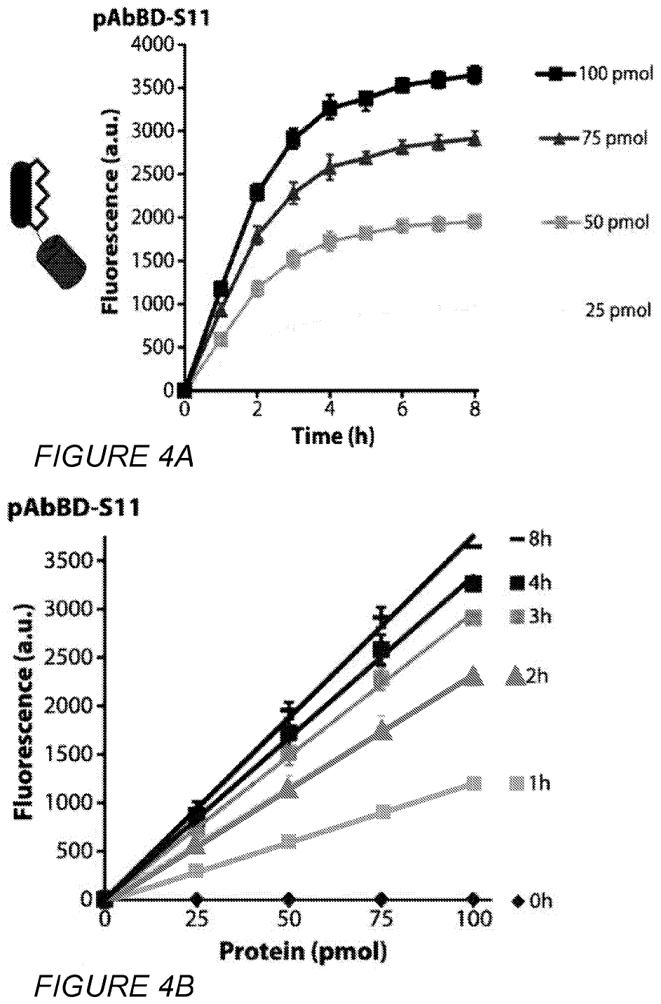

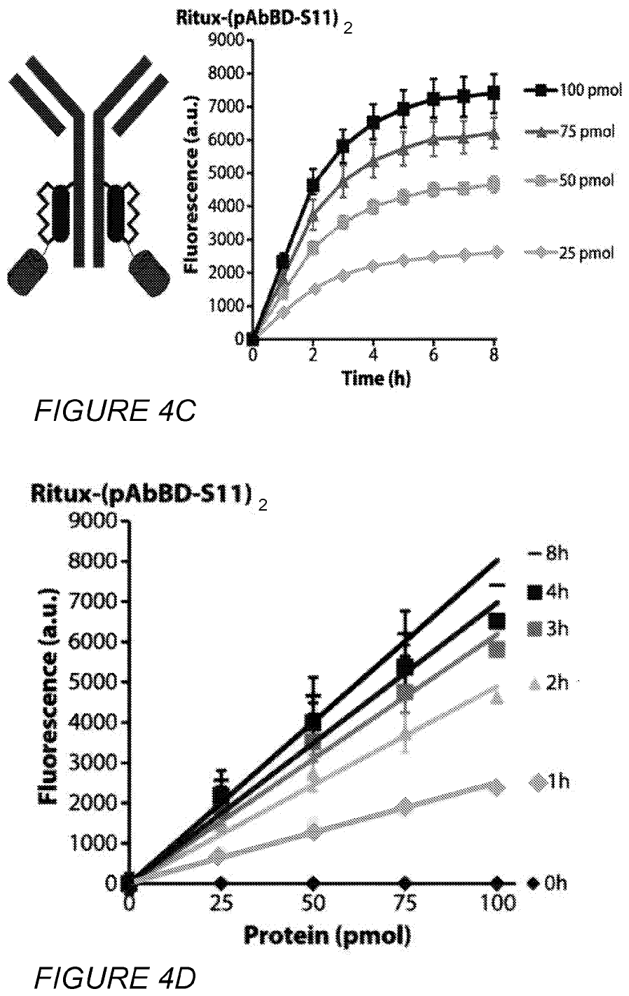

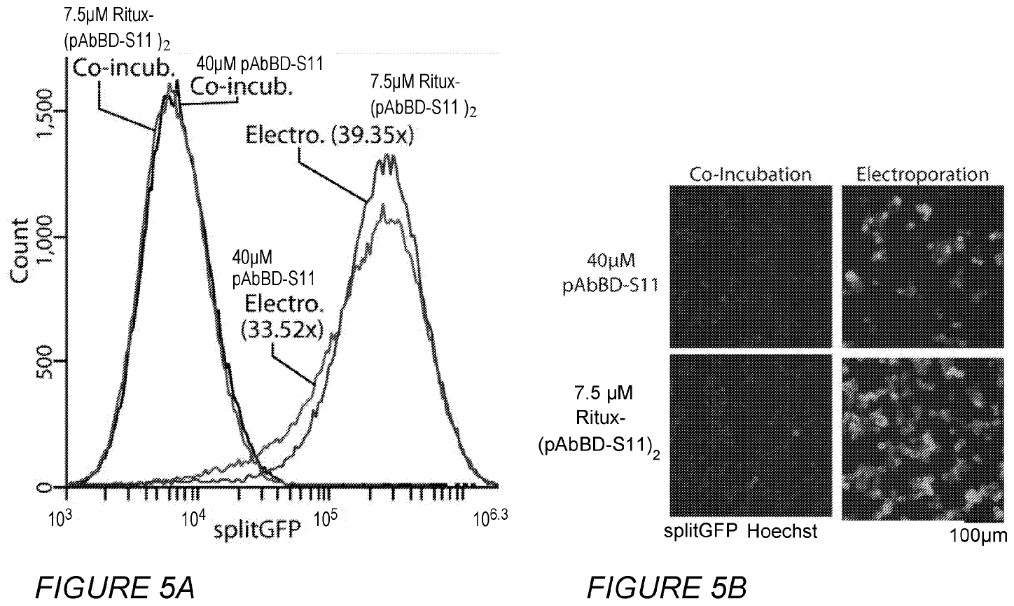

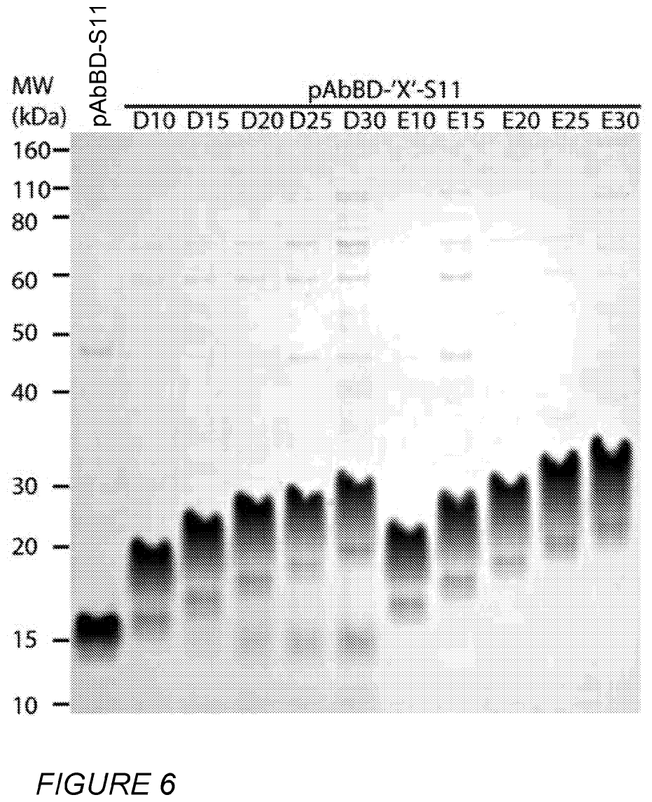

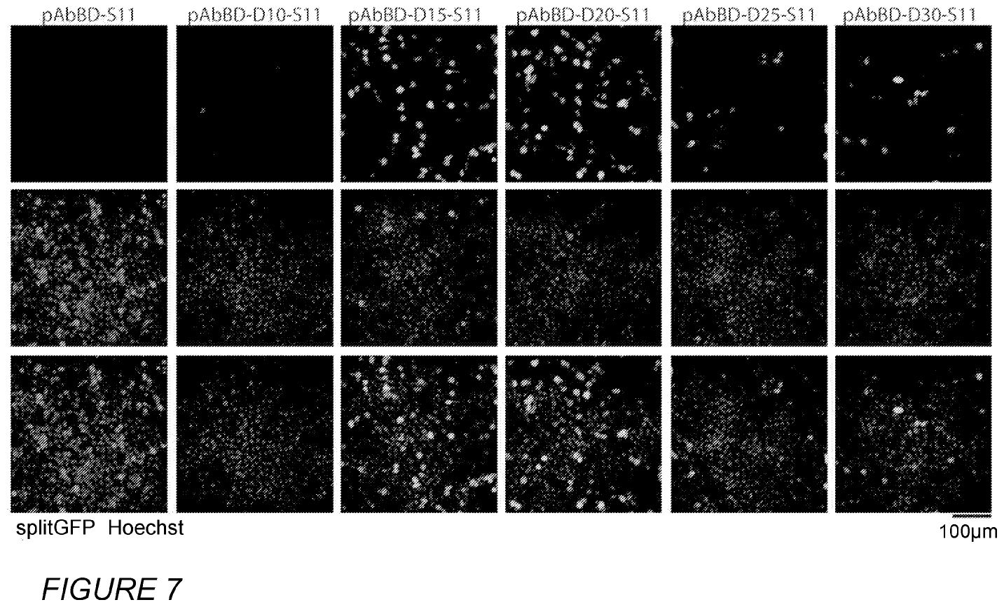

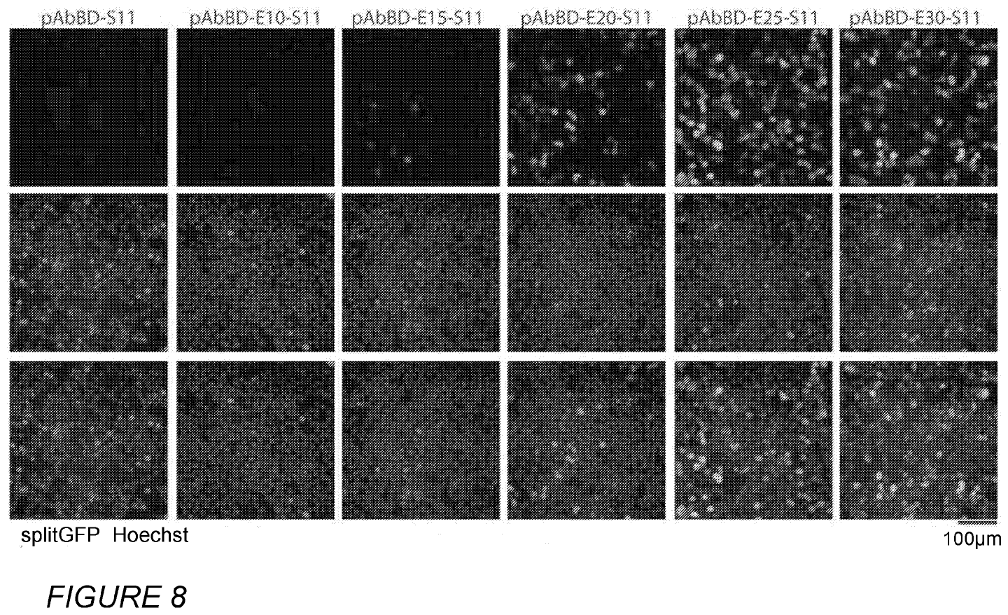

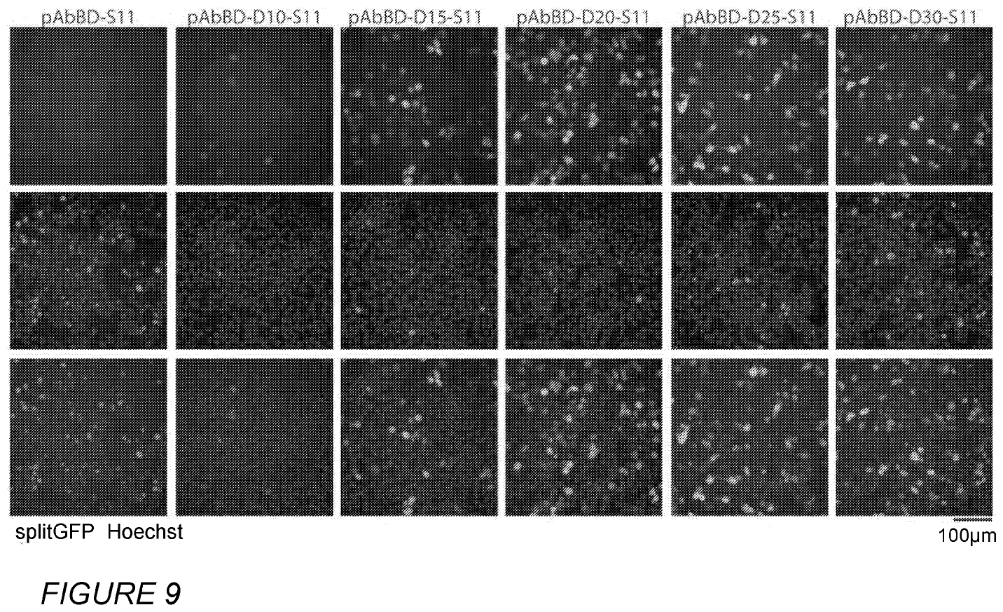

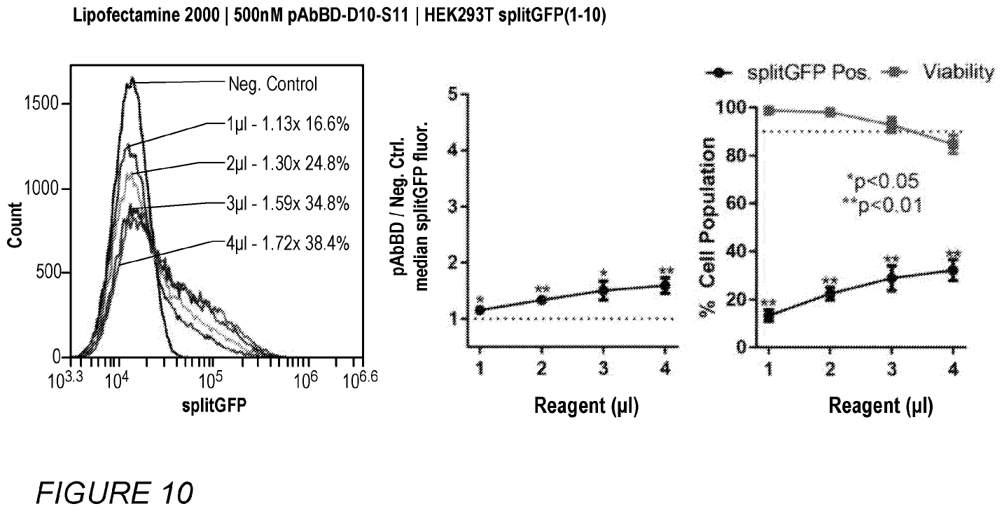

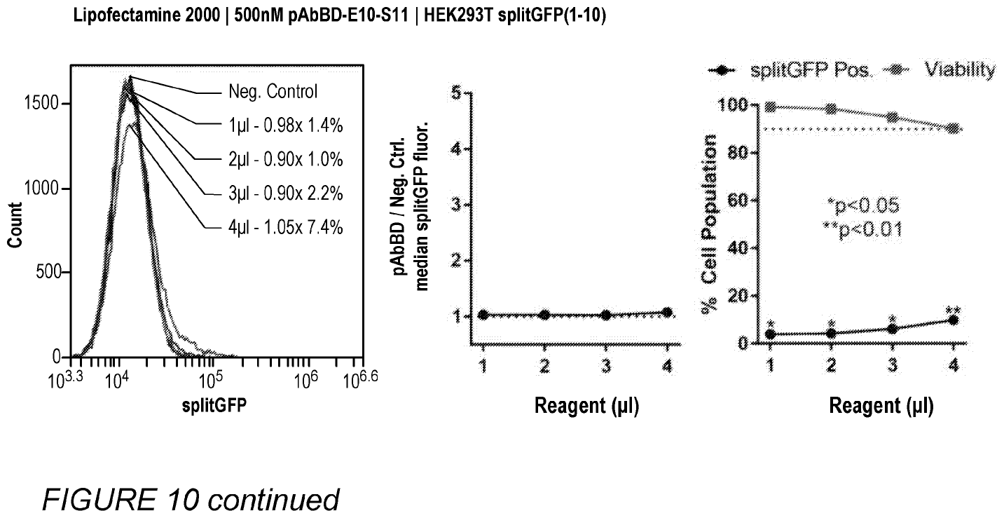

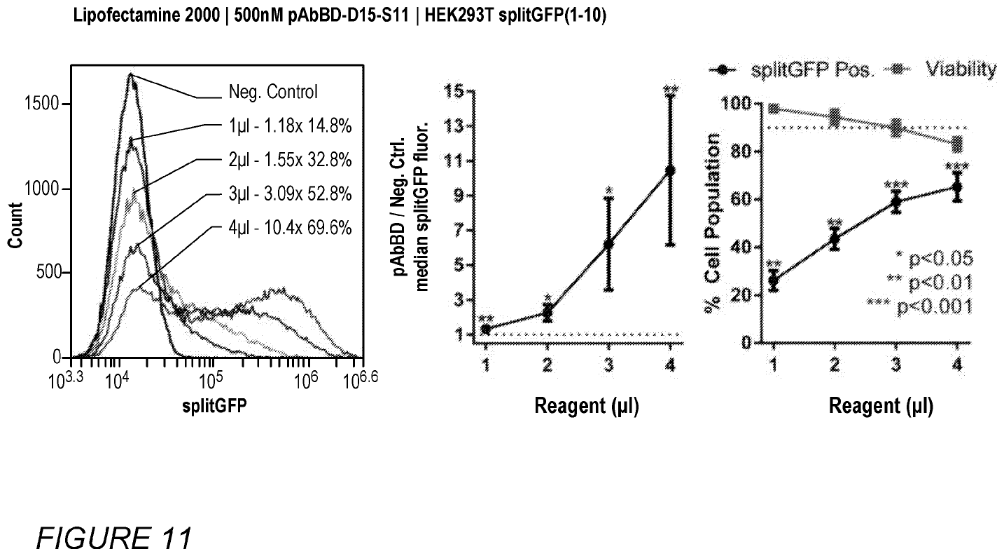

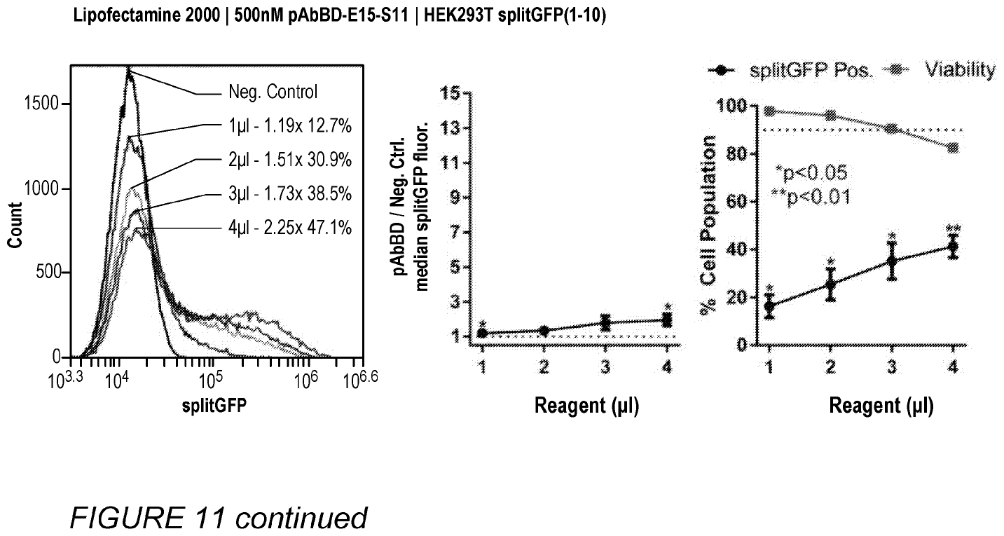

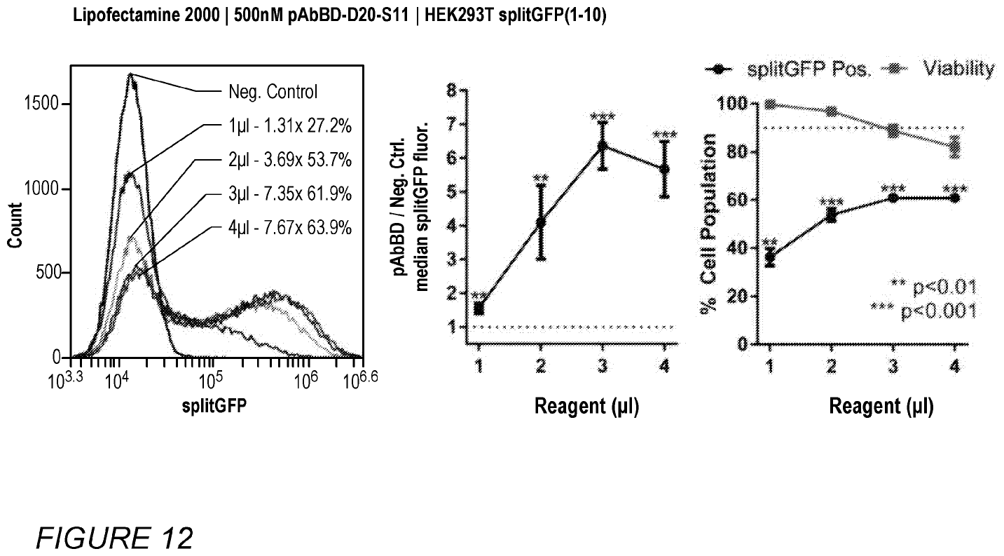

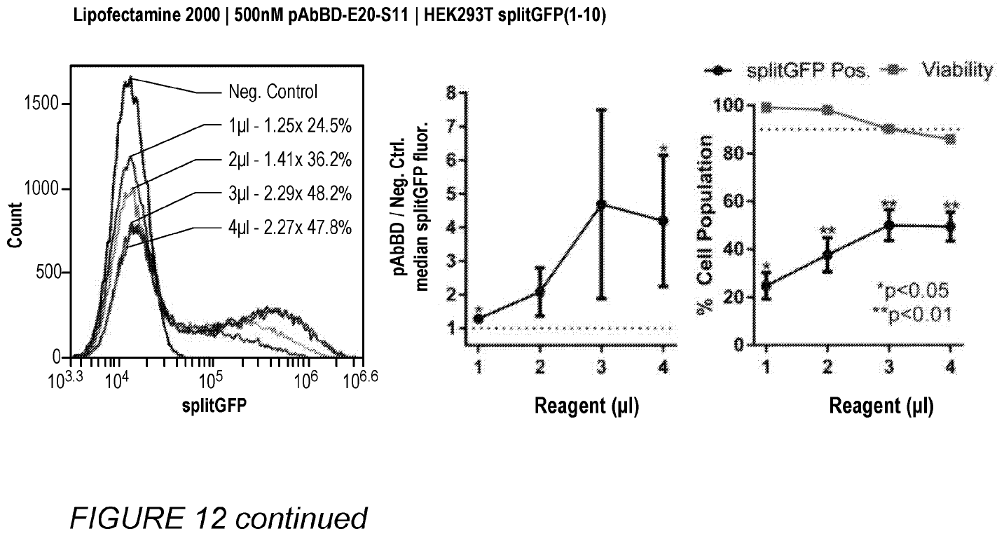

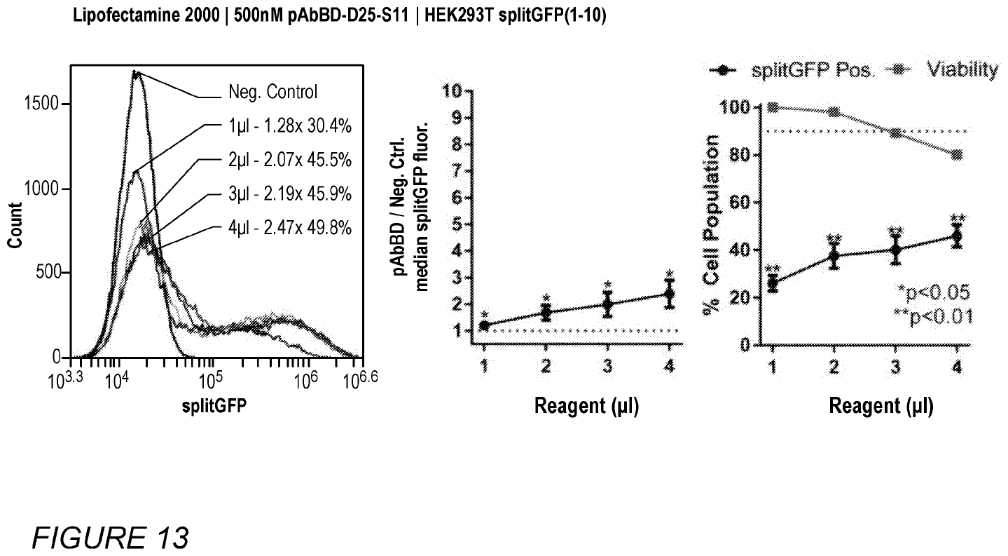

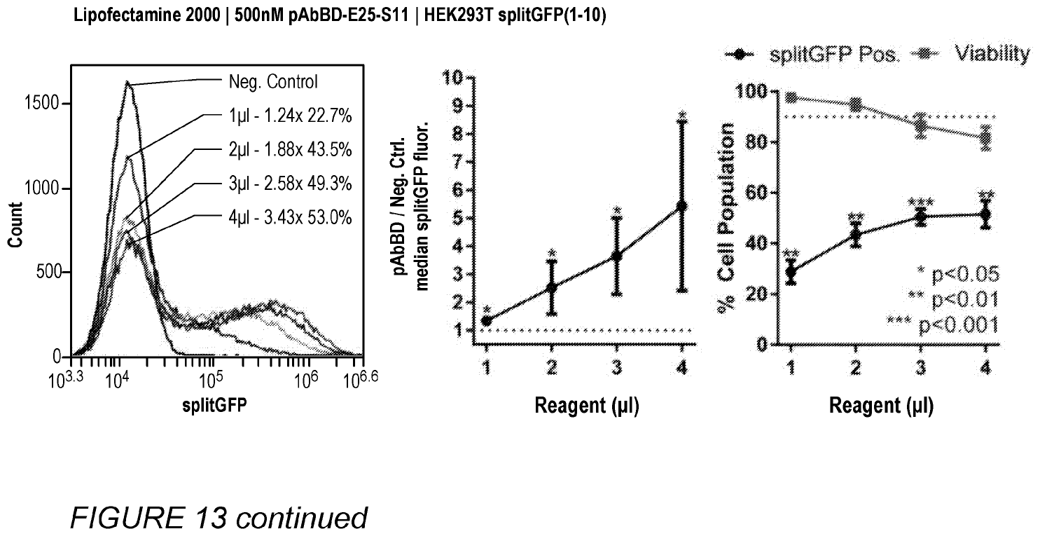

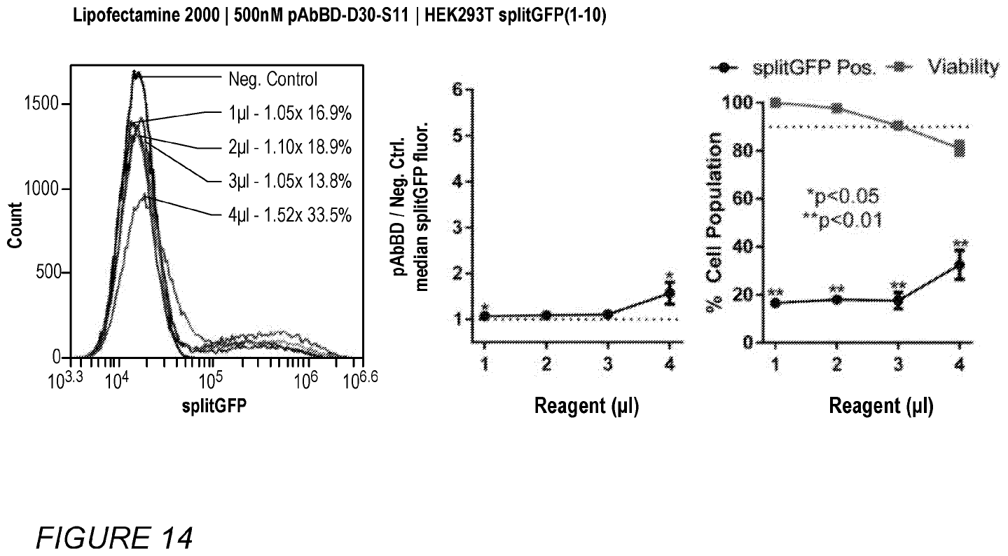

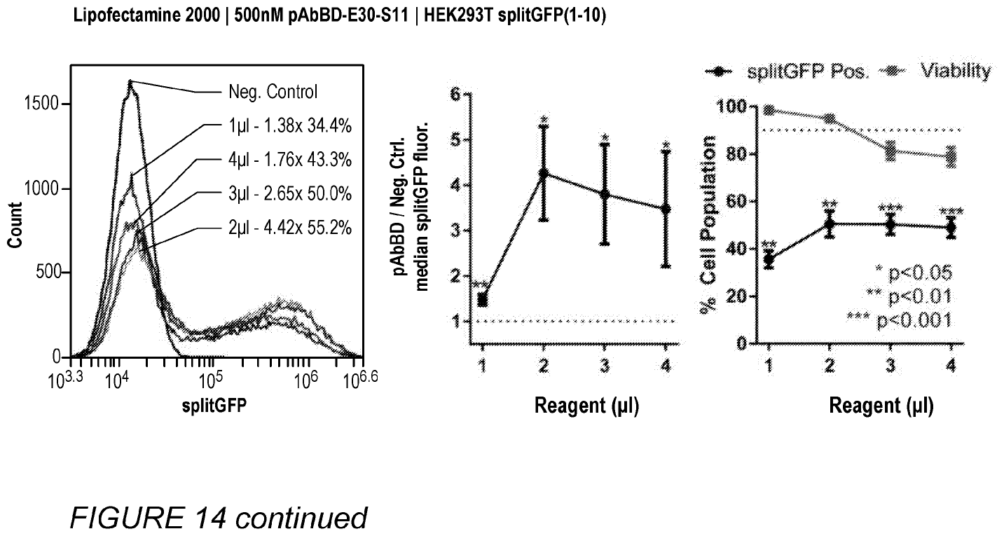

Preparation of pAbBD-ApP-S11 fusion proteins: Additional pAbBD-ApP-S11 fusion proteins can be prepared with 35 and 40 aspartic acid residues. Even longer polypeptides can be prepared if an upward trend in transfection efficiency continues to be seen. All fusion proteins can be expressed in a Proximity-Based Sortase-mediated Ligation (PBSL) system (FIG. 2). PBSL provides us with the flexibility to site-specifically label the C-terminus of the pAbBD-ApP-S11 fusion proteins with a fluorescent dye (i.e. Cγ5, which is optically distinct from GFP) so that cellular uptake and cytosolic delivery can be monitored independently. Specifically, complementation of the splitGFP S11 peptide with splitGFP(1-10) enables cytosolic delivery to be monitored, while the fluorescently labeled pAbBD-ApP-S11 fusion protein can help monitor total cell uptake. Even when no label is introduced, PBSL still allows proteins to be isolated with significantly higher purity than conventional purification systems due to the very mild, sortase-mediated elution conditions (i.e. calcium and triglycine). All of the pAbBD-ApP-S11 fusion proteins created to date are already produced using this PBSL system.

Antibody Conjugations: The pAbBD-ApP-S11 fusion proteins can be crosslinked to IgG, as previously described. The reaction products can be analyzed on a reducing and non-reducing PAGE to confirm specific labeling of the heavy chains. Unconjugated pAbBD-ApP-S11 fusion proteins can be removed using ultrafiltration spin columns (100 kDa MWCO, Millipore). Filtration can be conducted using Protein A/G elution buffer, to ensure only covalently bound pAbBD-ApP-S11 remains in the retentate. After washing, samples will be returned to PBS, pH 7.4. Purity of the resulting IgG-ApP-S11 conjugates can be evaluated by PAGE, FPLC, and mass spectrometry.

Optimization of cytoplasmic antibody delivery: HEK293T splitGFP(1-10), A549 splitGFP(1-10), and HT1080 split GFP(1-10) cells can be seeded onto a 48 well plate and incubated overnight. The IgG-ApP-S11 can be complexed with cationic lipid from various commercial vendors (e.g., Lipofectamine 2000, Lipofectamine 3000, RNAiMax, CRISPRMax, FuGENE, ViaFect, etc.) according to the manufacturer's instructions. A range of IgG:transfection reagent ratios can be tested. Rituximab can be used as a model IgG for all optimization experiments. The lipid-IgG complexes can be added to the cells at a final concentration between 5 nM and 1 μM IgG and incubated for 1 to 24 hours at 37° C. before determining the amount of splitGFP complementation by flow cytometry and fluorescence microscopy. Cγ5 fluorescence can also be analyzed to assess total cellular uptake and intracellular distribution. Co-localization of Cγ5 and GFP can be evaluated using ImageJ. Parallel studies can be performed to assess viability by LDH assay, for each of the experimental conditions tested. Negative control cells can undergo the same procedure, but with IgG conjugated to pAbBD-S11 protein, i.e. no ApP.

Characterization of antibody-mediated MRP1 inhibition via calcein assay: Anti-MRP1 antibodies (QCRL3; hybridoma acquired from ATCC) or isotype control antibodies (mIgG2a) can be delivered into HEK293T cells using optimal conditions, as determined from the above experiments (i.e. maximum delivery based on splitGFP fluorescence and >90% cell viability). The HEK293T cells can be loaded with calcein-AM at 37° C. for 30 minutes, after the IgG-lipid complex is washed from the cells. After the calcein-AM is removed and fresh media is added, the cells can be allowed to export calcein for 1 to 48 hours. Negative control cells can be treated with no IgG-lipid complex. Cells can be analyzed for fluorescence by flow cytometry and fluorescence microscopy, as a function of export time. If the median fluorescence of cells receiving QCRL3 is still elevated after 48 hrs, compared to cells treated with isotype control, one can test longer export times until no difference is observed to better understand the timeframe of inhibition. In addition to HEK293T cells, one can also test calcein export in A549, HT1080, and HEK293T cells that have been engineered to over express MRP1. Cell clones can be identified that express various levels of MRP1.

Antibody-mediated inhibition of drug export: Cell export assays can be performed with the chemotherapeutic drug doxorubicin (Dox) using the same procedure as described above with calcein; however, in addition to analysis of Dox fluorescence, cell viability can also be quantified via an MTT assay as a function of QCRL3 and mIgG2a concentration. Dose response curves and EC50 values can be determined using four-parameter curve fitting. Since Dox is a known substrate for MRP1, the EC50 will likely be lower for cells treated with QCRL3.

Characterization of antibody-mediated inhibition of Ras: The anti-Ras inhibitory antibody Y13-259, a non-inhibitory anti-Ras antibody Y13-238, and an isotype matched control IgG can be used to study the inhibition of Ras. Notably Y13-259 is a pan-Ras inhibitor that has previously been validated to inhibit Ras following microinjection. The hybridomas for both Y13-259 and Y13-238 are available from ATCC. Ras inhibition can be assessed by immunofluorescence staining for active phospho-Erk (ppErk) in A549 and HT1080 cells, which harbor activating mutations in K-Ras and N-Ras, respectively. A549 and HT1080 cells expressing splitGFP(1-10) can be seeded in 96-well plates and allowed to adhere overnight. IgG-ApP-S11 conjugates can be complexed with cationic lipids and delivered to cells under the optimized conditions determined above. After cytoplasmic delivery of IgG-ApP-S11, cells can be incubated in serum-free culture medium to remove external Ras-Erk pathway stimuli. After 24 h serum starvation, cells can be fixed, permeabilized, immunostained for active ppErk (pThr202/Tyr204), and probed with a secondary antibody conjugated to Alexa594. Cells can be imaged and analyzed for GFP and Alexa594. Efficacy of Ras inhibition can be assessed by examining single-cell distributions of ppErk intensity in cells that are GFP-positive. If GFP fluorescence is lost during processing, GFP-positive cells can be isolated by FACS and ppErk levels detected by Western Blot. Serum stimulation and Mek inhibition (30 min, 10 μM U0126) can be used as positive and negative controls, respectively. If cells do not survive 24 h of combined serum starvation and Ras inhibition, shorter starvation times can be tested. If Ras silencing is observed, results can be further validated by probing for Ras-dependent transcription targets (eg. Cyclin D1), proliferation (EdU incorporation, Ki67), and apoptosis (TUNEL, Annexin V).

Statistics: Statistical significance between groups/time points can be determined by analysis of variance (ANOVA) or a Student's t-test where appropriate. A p<0.05 can be considered statistically significant.

### Example 3

### IgG Conjugates that Target Intracellular Proteins for Degradation

While using antibodies to directly inhibit intracellular protein interactions or interfere with normal functionality can be an effective therapeutic strategy, the ability to target proteins for degradation could prove to be a more universal and modular approach, since inhibition would be independent of the binding epitope. Essentially any antibody with affinity for the target protein could be utilized. To target proteins for degradation, one can test two different destabilizing domains (DDs), FKBP12 and ecDHFR. These particular systems were selected because it has been shown that these DDs can be stabilized upon the administration of a small molecule. Degradation is only induced in the absence of this small molecule. This is advantageous because it will allow initiation of target protein degradation only after delivery has plateaued, which will allow the study of the degradation kinetics and the duration of degradation, independent of delivery. To monitor cytoplasmic delivery, IgG's will be labeled with pAbBDs that have been fused with the DDs as well as the ApP and S11 peptide. Constructs can be tested with the DD fused adjacent to the pAbBD at either the N- or C-terminus. To study the kinetics and duration of target protein degradation, one can remove the S11 peptide and study the degradation of GFP via flow cytometry and fluorescence microscopy, using an anti-GFP-DD-ApP conjugate. IgG-DD-ApP conjugates can also be evaluated in their ability to degrade MRP1 and Ras, using both inhibitory and non-inhibitory antibodies. These antibodies will allow evaluation to determine if there is any added benefit of using an inhibitory antibody in combination with a DD.

Preparation of pAbBD-DD-ApP fusion proteins and IgG-conjugates: Fusion proteins composed of a pAbBD, DD, ApP, and S11 peptide can be created. The DD can be fused adjacent to the pAbBD at either the N- or C-terminus. The ApP and S11 peptide can be fused at the C-terminus of the pAbBD-DD construct. Two different DDs can be tested, FKBP12 (˜12 kDa) and ecDHFR (˜18 kDa). Identical fusions proteins can be prepared without the S11 peptide. The pAbBD-DD fusion proteins can be crosslinked to IgG, as described above.

Optimization of antibody delivery: Optimization of cytoplasmic antibody delivery can be carried out as described herein, but with IgG conjugates that also include the DD domain (IgG-DD-ApP-S11). This will allow determination of whether the inclusion of the DD affects the efficiency of cytoplasmic delivery. A mouse IgG1 antibody can be used in these experiments, to match the anti-GFP antibody that can be used to study the kinetics of protein degradation. All cells can be continuously treated with either Shld1 or trimethoprim to stabilize FKBP12 and ecDHFR, respectively, and prevent the degradation of the IgG-DD-ApP-S11 conjugates. The time at which cytoplasmic antibody delivery peaks, after treatment with IgG-cationic lipid complexes, can be determined and used for subsequent degradation studies. Analogous studies can be performed to assess viability by an LDH assay, for each of the experimental conditions tested. Negative control cells can undergo the same procedure, but with IgG conjugated to pAbBD-S11 protein, i.e. no ApP.

Evaluation of GFP degradation kinetics and duration: IgG-DD-ApP conjugates (without the S11 peptide) can be prepared with an anti-GFP antibody (GFP-G1; hybridoma available from DSHB) and can be delivered into HEK293T-GFP, A549-GFP, and HT1080-GFP cells, based on conditions previously determined to be optimal. Notably, these cells can be engineered to stably express full-length GFP, not splitGFP(1-10). All cells can be continuously treated with either Shld1 or trimethoprim to stabilize FKBP12 and ecDHFR, respectively, during IgG delivery. At a time when cytoplasmic IgG levels are found to plateau, based on studies described above, the media can be replaced with fresh media that does not contain Shld1 or trimethoprim. GFP fluorescence can be analyzed by flow cytometry and fluorescence microscopy before and after removal of the Shld1 or trimethoprim, until GFP fluorescence returns to pre-treatment levels. This will allow study of the degradation kinetics and the duration of degradation, independent of delivery. Control studies may include cells treated with isotype control antibodies (mIgG1) and untreated cells. Analogous studies can be performed without Shld1 and trimethoprim.

Optimization and characterization of antibody-mediated degradation of MRP1-GFP: Non-inhibitory anti-GFP antibodies or isotype control antibodies (mIgG1) can be conjugated to pAbBD-DD-ApP fusion proteins and delivered into HEK293T cells that have been engineered to overexpress MRP1-GFP. Calcein export assays can be conducted as described above, except calcein blue-AM can be used, to avoid spectral overlap with GFP. Cells can be analyzed for fluorescence by flow cytometry and fluorescence microscopy, as a function of export time until no difference in calcein and GFP fluorescence is observed, between treated and untreated cells. Analogous studies can be performed with inhibitory anti-MRP1 antibodies (QCRL3) and isotype control antibodies (mIgG2a).

Antibody-mediated degradation of drug export: Cell export assays can be performed with the chemotherapeutic doxorubicin (Dox) using the same procedure as described above with Calcein blue-AM, except instead of fluorescence cell viability can be quantified via MTT assay as a function of antibody concentration. Dose response curves and EC50 values can be determined using four-parameter curve fitting.

Characterization of antibody-mediated degradation of Ras: Ras assays can be performed using the same procedure as described above, except with IgG-DD-ApP conjugates. Both an inhibitory anti-Ras antibody (Y13-259) and a non-inhibitory anti-Ras antibody (Y13-238) can be tested to see if there is any added benefit of using an inhibitory antibody in combination with a DD. Isotype control mIgG2a can be used as a negative control.

### Example 4

### Lipid-Like Nanoformulations for Targeted Delivery of Antibodies into Cells

Cytoplasmic delivery of antibodies into cells can be studied in living subjects. In particular, one can create a library of nanoparticle (NP) formulations comprised of a novel ionizable lipid-based material that was previously utilized for in vivo nucleic acid delivery, C12-200. This material consists of a chemically-modified polyamine (200) reacted with an epoxide-terminated carbon tail (C₁₂). The resulting branched, amine-rich ionizable material can facilitate efficient complexation with the IgG-ApP conjugates under acidic formulation conditions. Modification of polyamines with alkyl chains affords lipid-like properties, promoting NP formation through hydrophobic aggregation in aqueous conditions. Because of their lipid-like properties, polyethylene glycol (PEG)-lipids can be anchored into the surface of NPs, which acts to enhance NP serum stability and improve biodistribution in vivo. One can develop nanoparticles optimized for antibody delivery by creating a library of formulations that vary in terms of the amount and ratios of (i) phospholipid—in addition to the ionizable lipid material—which provides structure to the NP bilayer and can assist in endosomal escape, (ii) cholesterol, which enhances NP stability and promotes membrane fusion, and (iii) lipid-anchored PEG, which reduces NP aggregation and enhances biodistribution. The efficiency of cytoplasmic antibody delivery and the efficacy of Ras inhibition can be evaluated in both culture and in tumor bearing mice. The method of Ras inhibition, i.e. either direct target inhibition or target degradation, can be selected based on what was found to be the most potent approach.

Synthesis and characterization of lipid-like NPs: C12-200 can be synthesized by reacting C₁₂ epoxide-terminated lipids tails with a polyamine core (termed 200) at a 3:1 molar ratio at 90° C. in 100% ethanol for 48-72 hours. This material can be characterized by flash and thin layer chromatography, using matrix-assisted laser desorption ionization time of flight (MALDI-TOF) and ¹H-NMR spectroscopy. Upon confirmation of synthesizing the correct chemical structure, C12-200 can be combined with three excipients (phospholipid, cholesterol, and lipid-anchored PEG) and mixed with antibodies in a microfluidic device, used to induce chaotic mixing of the alcoholic lipid solution with aqueous antibody solution to produce NPs and promote entrapment of the IgG-ApP conjugates. To optimize NPs for antibody delivery, one can develop a library of NP formulations where one varies (i) the C12-200:antibody weight ratio, (ii) the phospholipid identity (including DSPC, DOPE, DOPC), and (iii) the molar composition of the four-component NP formulation (ionizable lipid, phospholipid, cholesterol, lipid-anchored PEG) using Design of Experiment optimization methodologies previously described. The pAbBD fusion protein to be utilized, for antibody conjugations, can be selected based on findings discussed above. In particular, one can select the approach, i.e. either direct target inhibition or target degradation, that leads to the most potent inhibition of normal Ras function. NP structure can be characterized by transmission electron microscopy, and NP size can be determined using dynamic light scattering (DLS). Antibody concentration can be measured using a Cγ5 label on the antibody. Lipid-like NPs have previously been shown to deliver a range of nucleic acids to tumors.

Evaluation of cytoplasmic antibody delivery: Optimization of cytoplasmic antibody delivery and cytotoxicity assays can be carried out as described above.

Characterization of antibody-mediated inhibition/degradation of Ras: Ras assays can be performed using the same procedure as described above.

Preparation of pAbBD-ApP-HiBit NanoLuc fusion proteins: To enable cytoplasmic delivery of antibodies to be monitored in living subjects, one can fuse HiBiT to the c-terminus of the pAbBD-ApP construct, in place of the S11 peptide. HiBiT is an 11-amino acid peptide that binds tightly to LgBiT (K_{D}=0.7 nM) to spontaneously form a bright, bioluminescent enzyme, NanoLuc (Promega). IgG conjugates can otherwise be prepared in an identical manner. HEK293T, A549, and HT1080 cells can be engineered to express the LgBiT enzyme to enable cytoplasmic delivery to be monitored by bioluminescence. This system can be validated in culture by comparing the kinetics of cytoplasmic delivery to analogous studies with the splitGFP system.

Bioluminescent analysis of cytoplasmic antibody delivery in tumor-bearing mice: A dose-ranging study can be performed using a 4-log range of IgG-NPs to determine the approximate dose needed to achieve cytoplasmic delivery, based on bioluminescent measurements. The IgG-NPs can be prepared using a non-targeted antibody (i.e. Rituximab) and pAbBD-ApP-HiBiT constructs without a DD domain. A549-LgBiT cells can be subcutaneously implanted into ˜6-week old nude mice (n=50). Once tumors reach ˜5 mm, mice can be randomly placed into 5 IgG dose groups (PBS-only, 0.05 mg/kg, 0.5 mg/kg, 5 mg/kg, and 50 mg/kg; n=10 mice/group). The IgG-NPs can be delivered i.v. over the course of 5 days (one injection per day). Mice can be evaluated daily for bioluminescence, weight, activity, well-being, and overall survival. Tumor growth can be measured with a caliper and tumor volume calculated using an ellipsoid formula. Bioluminescence can be monitored for at least 1-week after the first injection and until no bioluminescence in the tumor can be detected, compared with surrounding muscle. The mice can be sacrificed at this time and the blood, tumor, lungs, liver, spleen, bladder, heart, kidneys, and brain can be harvested. A standard curve with IgG-HiBiT conjugates in the presence of LgBit can be established in vitro and used to estimate the percent injected IgG dose that is cytoplasmically delivered in vivo. Additional animals (n=10), that received the highest dose, can be sacrificed 24 h after the final injection, so that the accumulation and distribution of IgG in the tumors can be assessed by immunostaining. Harvested tissues can be examined by a veterinary pathologist (Penn Comparative Pathology Core) blinded to the treatment groups, to assess for potential effects of the NPs on organ morphology and function. Whole blood analysis can also be performed including white blood cell count, hemoglobin, platelet count, neutrophils, lymphocytes, monocytes, blood urea nitrogen, alkaline phosphatase, aspartate aminotransferase, alanine aminotransferase and creatine phosphokinase. Tumors can be sectioned and immunostained for the presence of Rituximab using anti-human antibodies.

Targeting Ras in tumor-bearing mice: Approximately 6-week old nude mice (n=36) can have A549 cells implanted subcutaneously. Once tumors reach ˜5 mm, mice can be further sub-divided into 3 groups (PBS-only, Inhibitory IgG-NPs, non-inhibitory IgG-NPs; n=12 mice/group). The IgG-NPs can be delivered i.v. over the course of 5 days (one injection per day). The IgG conjugates utilized can be based on the formulation from Examples 2 or 3 that leads to the most potent inhibition of normal Ras function. Mice can be evaluated daily for bioluminescence, weight, activity, and well-being, and overall survival (out to 30 days post-treatment). Tumor growth can be measured with a caliper and tumor volume can be calculated using an ellipsoid formula. A log-rank analysis can be performed on data in Kaplan-Meier curves (generated from survival data) to identify statistical significance (p<0.05) between groups. At time of sacrifice, the blood, mammary glands, lungs, liver, spleen, bladder, heart, kidneys, and brain can be harvested and analyzed by the Penn Comparative Pathology Core as described above. Additional animals (n=36) that receive PBS-only, Inhibitory IgG-NPs, or non-inhibitory IgG-NPs (n=12 mice/group) can be sacrificed 24 h after the final injection, so that the Ras inhibition can be assessed by immunostaining. Specifically, all tumors can be sectioned and immunostained for the presence of ppErk.

### Example 5

### Cytosolic Anti-RelA IgG Delivery Inhibits NFκB Transcriptional Activity

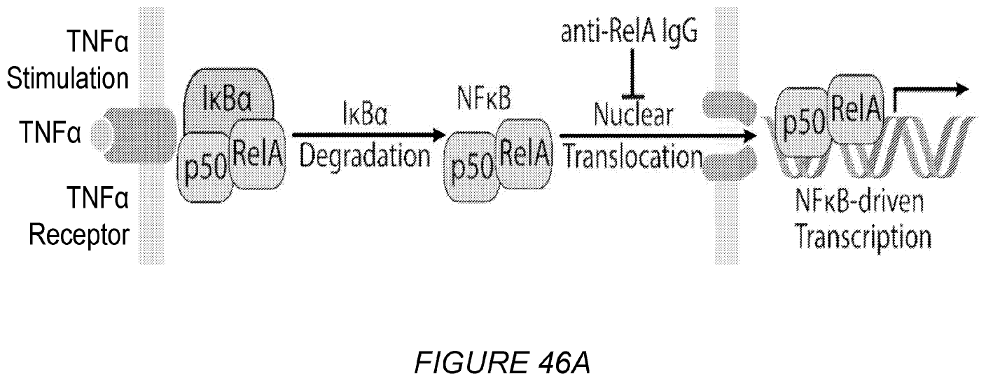

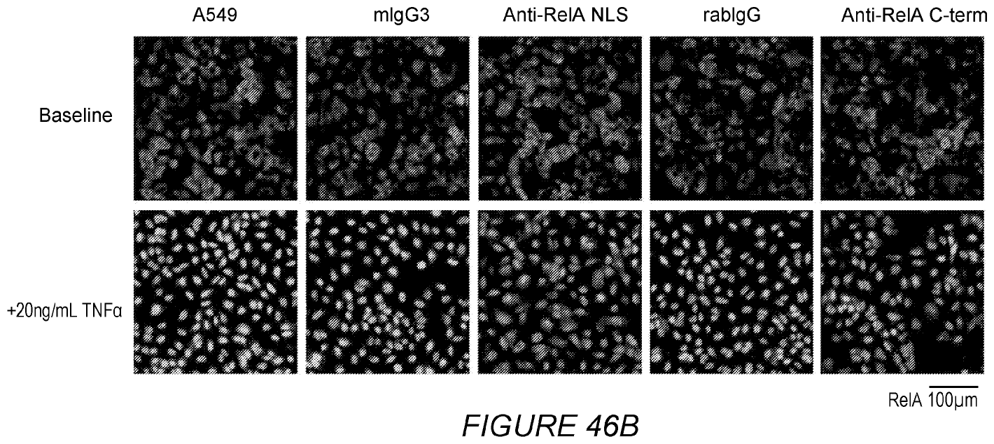

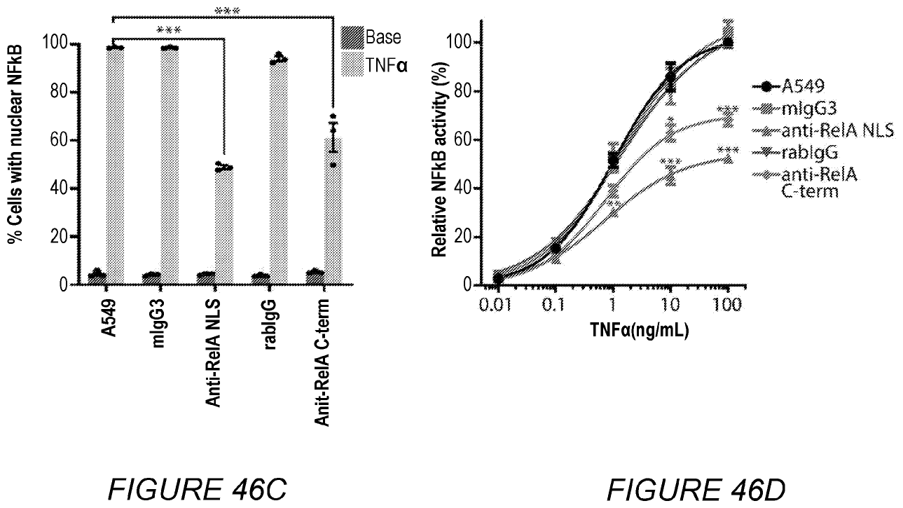

The RelA (also known as p65) gene encodes the human protein transcription factor p65 (also known as nuclear factor NF-kappa-B p65 subunit). RelA is a member of the NF-κB transcription factor complex. NF-kB-mediated signaling has roles in inflammatory and immune responses; abnormal NF-kB activity has also been associated with cancers, including solid tumors and hematologic cancers, such as acute and chronic leukemias, lymphomas, multiple myeloma and myelodysplastic syndromes, and promoting tumor growth. A549 is a cancer cell line, specifically an adenocarcinomic human alveolar basal epithelial cell line, which is used to study lung cancer and to testanti-cancer drugs in vitro and in vivo via xenografting. Anti-RelA antibodies, such as anti-RelA IgGs inhibit NF-kB transcriptional activity by preventing its nuclear translocation following TNFα stimulation, as shown in the schematic of FIG. 46A. 150 nM of the following antibody conjugates were delivered to the cytosol of A549 cells: 150 nM IgG-(pAbBD-D25-S11)₂ antibody, mIgG3-(pAbBD-D25-S11)₂ (anti-RelA NLS isotype control), anti-RelA NLS IgG-(pAbBD-D25-S11)₂, rabIgG-(pAbBD-D25-S11)₂ (anti-RelA C-term isotype control) or anti-RelA C-term IgG-(pAbBD-D25-S11)₂. FIGS. 46B-46C show representative immunofluorescence images (FIG. 46B) and quantification (FIG. 46C) of RelA nuclear translocation following delivery of the indicated 150 nM IgG-(pAbBD-D25-S11)₂ antibody and TNFα treatment. Only cytosolic delivery of the anti-RelA IgGs reduced RelA nuclear translocation. Data are mean±s.e.m, n=3, ***p<0.001 (one-way ANOVA).

FIG. 46D shows A549 cells that were transiently transfected with a NFκB-driven firefly luciferase reporter plasmid. NFκB transcriptional activity was detected by luminescence following delivery of the indicated 150 nM IgG-(pAbBD-D25-S11)₂ antibody and TNFα treatment. Only delivery of anti-RelA IgGs inhibited NFκB transcriptional activity. Data are mean±s.e.m, n=3, *p<0.05 **p<0.01 ***p<0.001 (one-way ANOVA).

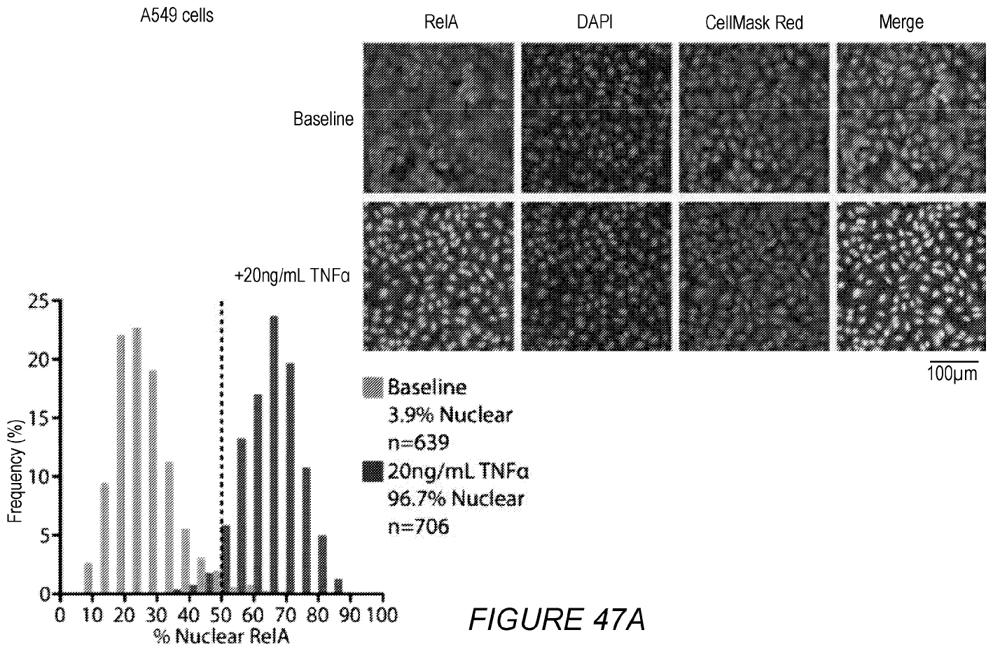

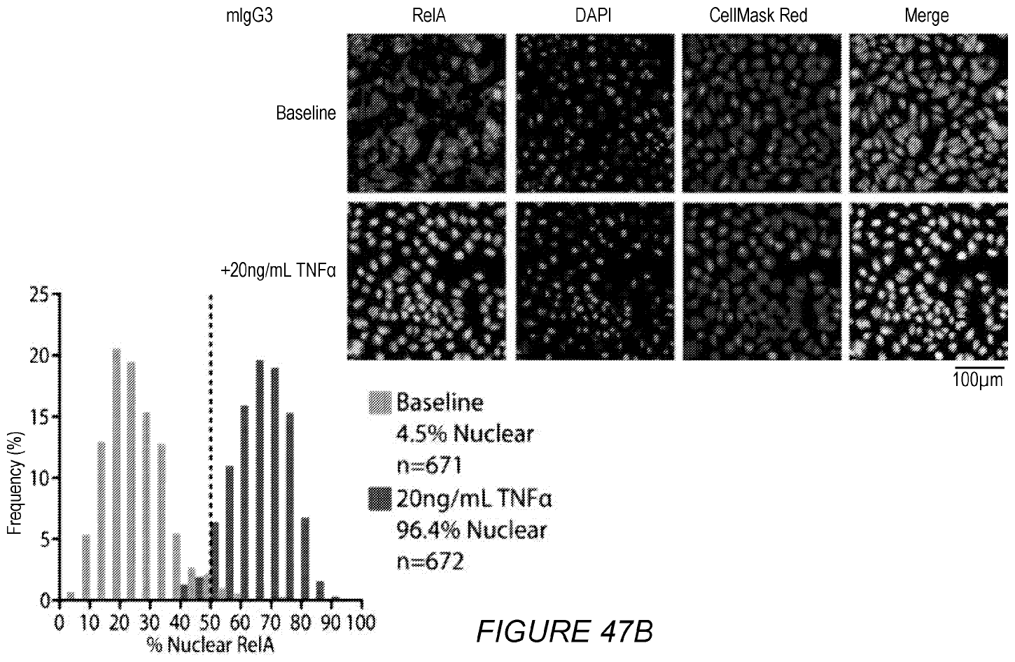

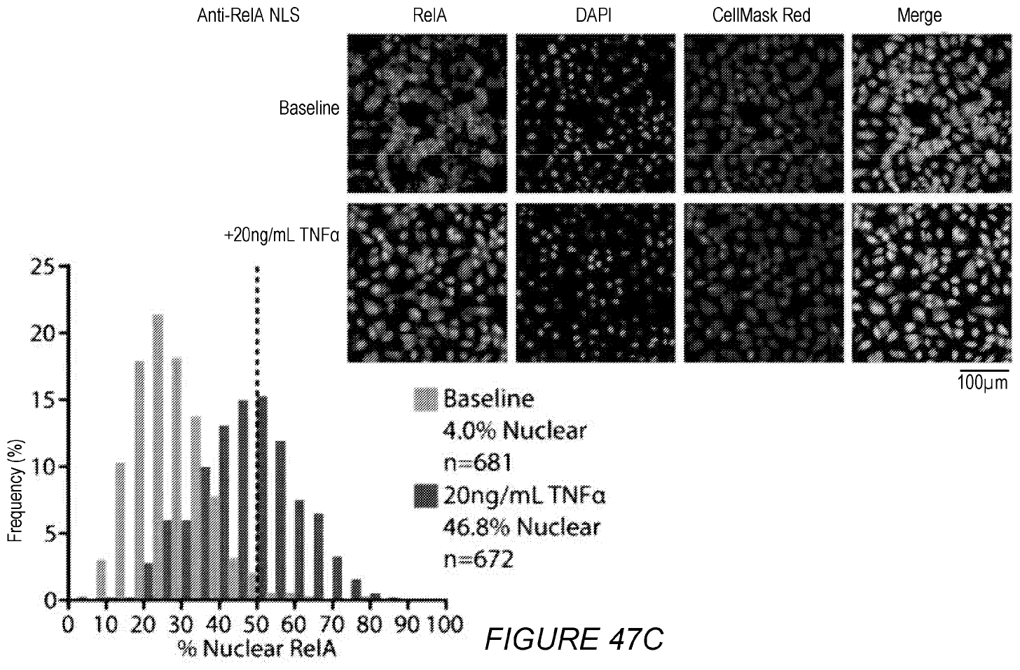

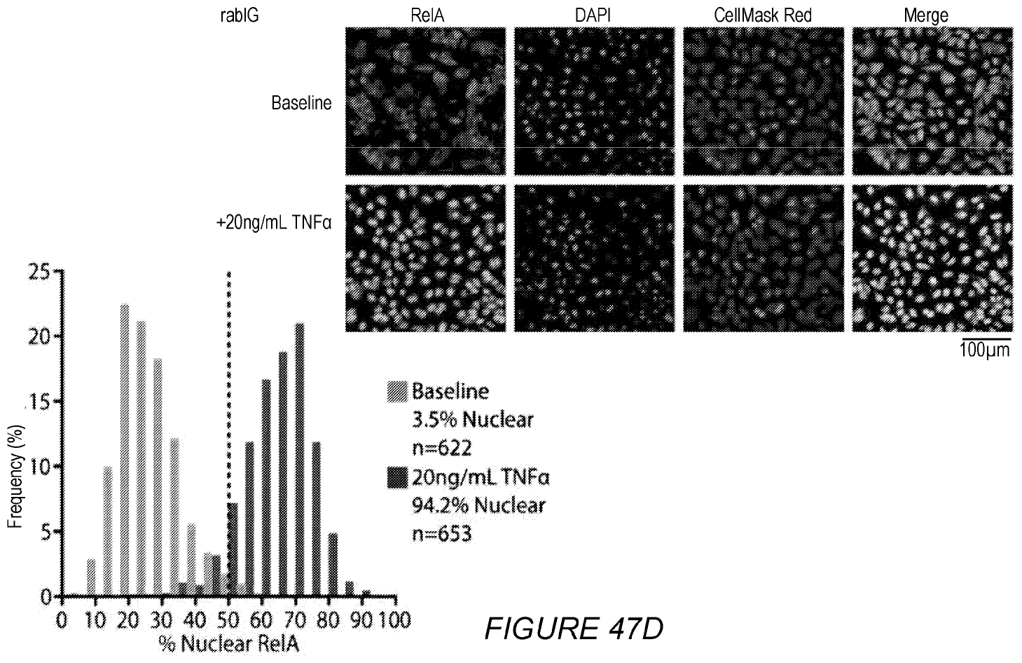

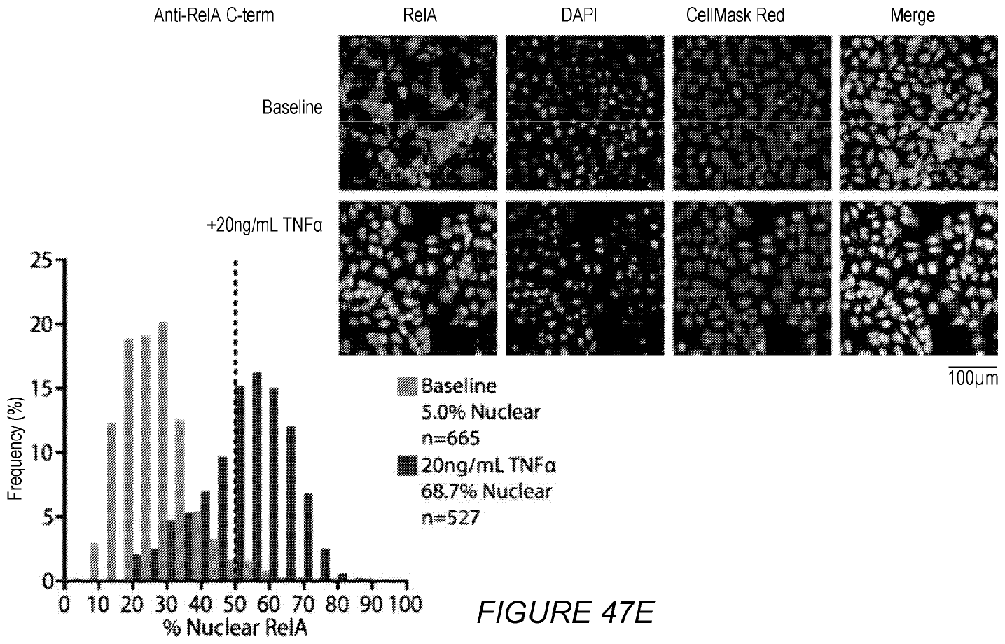

FIGS. 47A-47E relate to FIG. 46 and show RelA immunofluorescence quantification and are related to FIGS. 46A-46D. FIGS. 47A-47E show representative immunofluorescence images of A549 cells with or without TNFα stimulation are shown without protein delivery (FIG. 47A) or with 150 nM mIgG3-(pAbBD-D25-S11)₂ (anti-RelA NLS isotype control) (FIG. 47B), anti-RelA NLS IgG-(pAbBD-D25-S11)₂ (FIG. 47C), rabIgG-(pAbBD-D25-S11)₂ (anti-RelA C-term isotype control) (FIG. 47D), or anti-RelA C-term IgG-(pAbBD-D25-S11)₂ (FIG. 47E) delivered with 2 μl Lipo RNAiMax. CellProfiler was used for automated image analysis. The DAPI channel was used for nuclear segmentation whereas the CellMask Red channel was used for cellular segmentation. For each cell, the nuclear RelA fluorescence intensity was normalized to the cellular RelA fluorescence intensity. At least 5 image sets were taken for each biological replicate. Histograms of normalized nuclear RelA fluorescence are shown for one biological replicate for each delivery condition. 50% nuclear RelA was used as a cutoff for denoting a cell as having nuclear RelA.

### Example 6

### Cytoplasmic Delivery of Proteins Other than pAbBD and IgGs: Single Chain Targeting Ligands

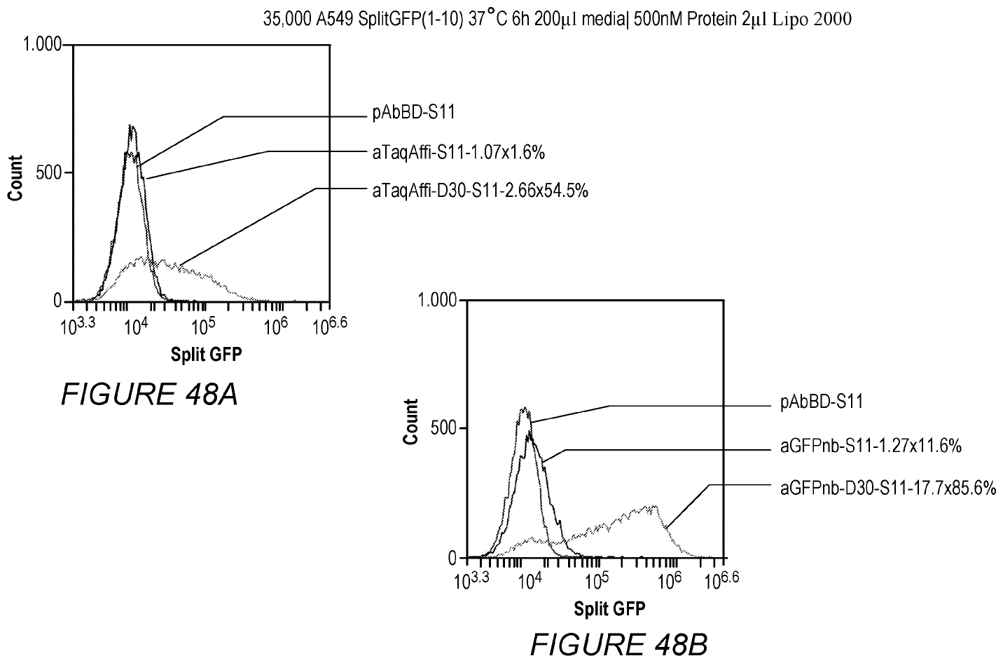

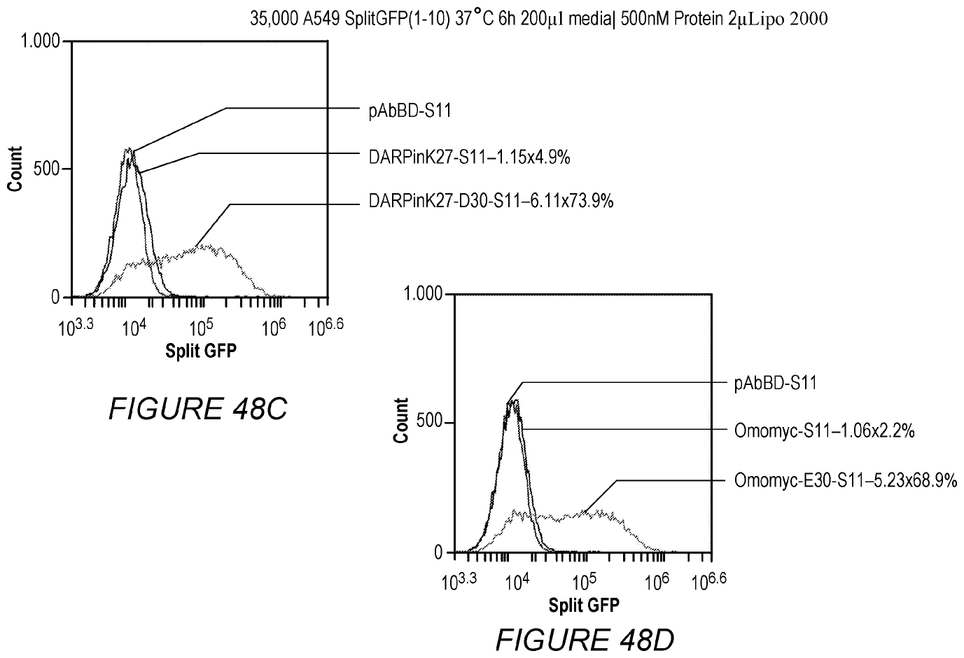

35,000 A549 splitGFP(1-10) cells were seeded onto each well of a 48 well plate in 180 μL media at 37° C. for 12-16 hours. Lipid nanoparticles were formed by incubating 2 μl Lipofectamine 2000 with 500 nM anti-Taq affibody, anti-GFP nanobody, DARPinK27, or Omomyc with either no ApP or the indicated ApP in OptiMEM (20 μL final volume, pH 7.4) at 25° C. for 10 min., as indicated in FIG. 48. The lipid nanoparticles were then added to the cells and incubated for 6 hours at 37° C. before determining the amount of splitGFP complementation by flow cytometry. Negative controls undergo the same procedure, but with 500 nM pAbBD-S11 protein. Representative flow cytometry histograms of splitGFP fluorescence are shown in FIG. 48. For each protein, the percent of cells splitGFP-positive and the fold-increase in median splitGFP fluorescence over negative control (pAbBD-S11) are indicated in FIG. 48.

### Example 7

### Cytoplasmic Delivery of DARPinK27 can Inhibit KRas-G12C Signaling in A549 Cells

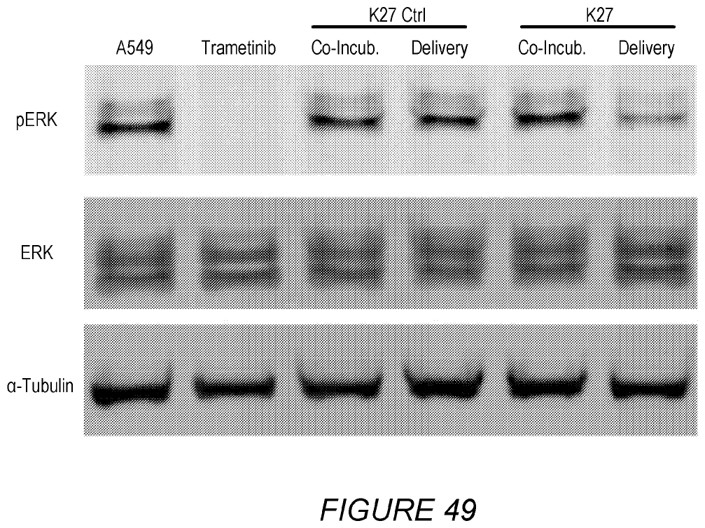

DARPinK27 is a synthetically designed protein capable of binding to and inhibiting KRas activity. 500 nM DARPinK27-D₃₀-S11 and a negative control DARPinK27n3-D₃₀-S11 were either co-incubated or cytosolically delivered into A549 cells with Lipo 2000. 4 hours following delivery, KRAs signaling activity was determined by stimulating A549 cells with 100 ng/mL hEGF for 30 min. and then western blotting for phosphorylated ERK. 100 nM Trametinib treatment was used as a positive control. α-Tubulin was used as a loading control, as shown in FIG. 49. As expected, treating cells with Trametinib, a MEK inhibitor, abolished ERK phosphorylation, but cytoplasmically delivering DARPinK27n3, which is incapable of binding to KRas, or incubating cells with either the control or active DARPinK27 did not affect pERK levels. Cytoplasmic delivery of 500 nM DARPinK27-D₃₀-S11 reduced pERK levels by ˜65%, indicating successful inhibition of KRas-G12S signaling.

### Example 8

### pAbBD-S11-Oligonucleotide Conjugate Delivery into A549 SplitGFP(1-10) Cells

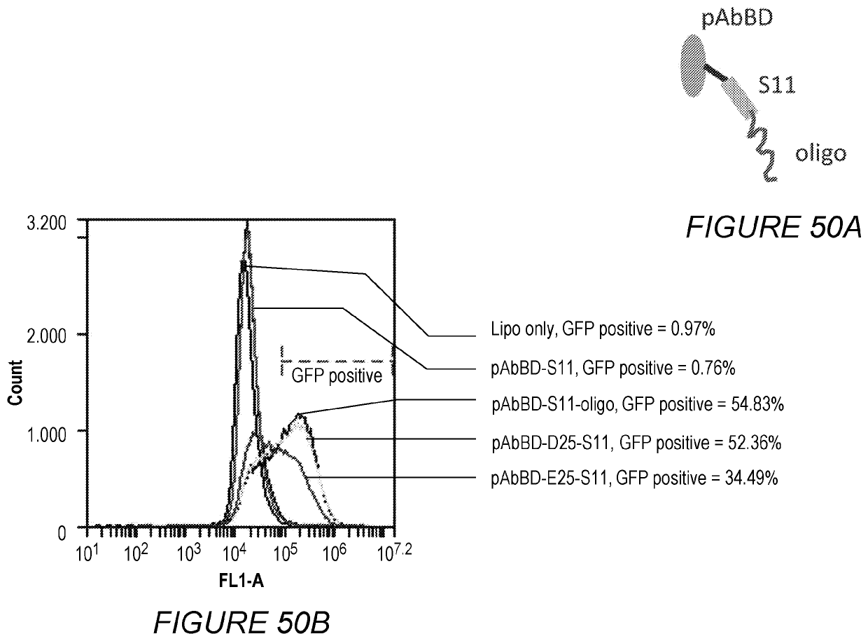

A pAbBD-S11 fusion protein was conjugated to an anionic nucleic acid (an oligonucleotide), as shown in the schematic of FIG. 50A. The pAbBD-S11-oligo conjugate was complexed with lipofectamine 2000 and delivered into A549 splitGFP(1-10) cells. Lipid nanoparticles were formed by incubating 2 μl Lipofectamine 2000 with 500 nM pAbBD-S11-oligo, pAbBD-D25-S11, or pAbBD-E25-S11 in OptiMEM (20 μL final volume, pH 7.4) at 25° C. for 10 min. The lipid nanoparticles were then added to the cells and incubated for 6 hours at 37° C. before determining the amount of splitGFP complementation by flow cytometry. Negative controls undergo the same procedure, but with 500 nM pAbBD-S11 protein or lipid only. Representative flow cytometry histograms of splitGFP fluorescence are shown in FIG. 50B. For each protein, the percent of cells splitGFP-positive are indicated in FIG. 50B.

### Example 9

### pAbBD-S11-Oligonucleotide Cell Delivery with Lipofectamine 2000

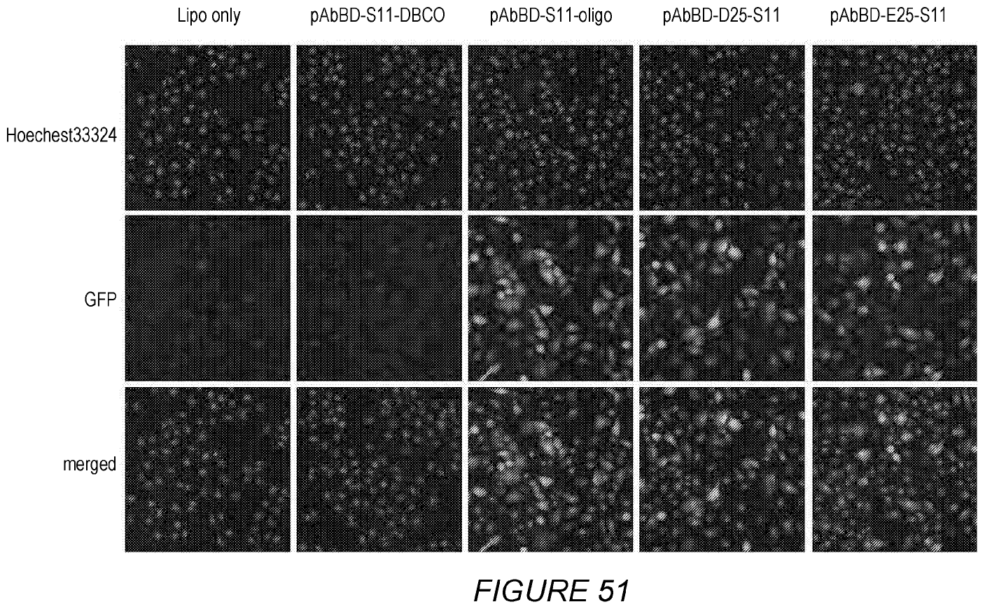

Lipid nanoparticles were formed by incubating 2 μl Lipofectamine 2000 (“lipo”) with 500 nM pAbBD-S11-oligo, pAbBD-D25-S11, or pAbBD-E25-S11 in OptiMEM (20 μL final volume, pH 7.4) at 25° C. for 10 min. Lipo only and pAbBD-S11 labeled at the c-terminus with a DBCO (“pAbBD-S11-DBCO”), complexed with Lipo, were used as negative controls. The lipid nanoparticles were added to the cells and incubated for 6 hours at 37° C. before live cell fluorescence microscopy. 50 μg/mL Hoechst 33342 was added 30 minutes prior to microscopy. In FIG. 51, the top channel is the Hoechst channel, the middle panel is the splitGFP channel, and the bottom panel is the split GFP and Hoechst channel merged.

### Example 10

### Delivery of Light Activated Site-Specific Conjugate of IgG with a pAbBD-S11 Fusion Protein Conjugated to an Oligonucleotide

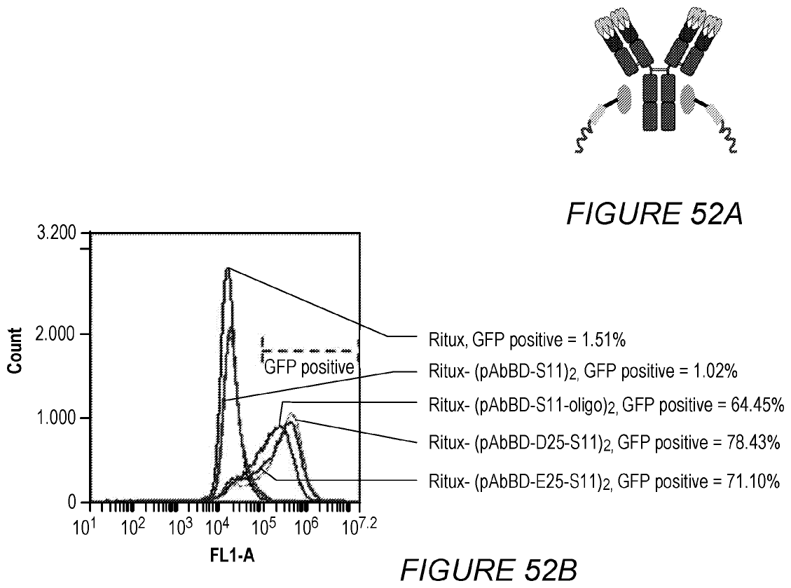

A light activated site-specific conjugate of IgG with a pAbBD-S11 fusion protein conjugated to an anionic nucleic acid (an oligonucleotide) is shown in the schematic of FIG. 52A. Rituximab (Ritux) was used as a model IgG to conjugate to the fusion protein pAbBD-S11-oligo)₂ to make the conjugate Ritux-(pAbBD-S11-oligo)₂. Ritux-(pAbBD-S11-oligo)₂ was complexed with lipofectamine 2000 and delivered into A549 splitGFP(1-10) cells. Lipid nanoparticles were formed by incubating 2 μl Lipofectamine 2000 with 500 nM Ritux-(pAbBD-S11-oligo)₂, Ritux-(pAbBD-D25-S11)₂, or Ritux-(pAbBD-E25-S11)₂ in OptiMEM (20 μL final volume, pH 7.4) at 25° C. for 10 min. The lipid nanoparticles were then added to the cells and incubated for 6 hours at 37° C. before determining the amount of splitGFP complementation by flow cytometry. Negative controls undergo the same procedure, but with 500 nM Ritux-(pAbBD-S11)₂ protein or Ritux only. Representative flow cytometry histograms of splitGFP fluorescence are shown in FIG. 52B. For each protein, the percent of cells splitGFP-positive are indicated.

### Example 11

### Cell Delivery of Ritux—pAbBD-S11-Oligonucleotide Conjugates with Lipofectamine 2000

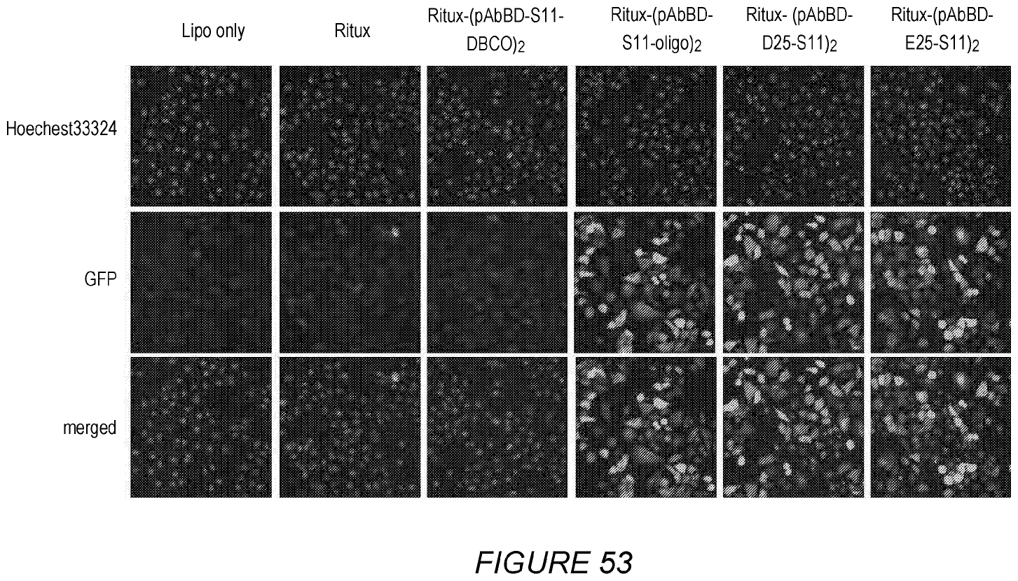

A schematic depicting the light activated site-specific conjugation of IgG with a pAbBD-S11 fusion protein conjugated to an anionic nucleic acid (an oligonucleotide) is shown in FIG. 52A. Ritux-(pAbBD-S11-oligo)₂ was complexed with lipofectamine 2000 and delivered into A549 splitGFP(1-10) cells. Lipid nanoparticles were formed by incubating 2 μl Lipofectamine 2000 with 500 nM Ritux-(pAbBD-S11-oligo)₂, Ritux-(pAbBD-D25-S11)₂, or Ritux-(pAbBD-E25-S11)₂ in OptiMEM (20 μL final volume, pH 7.4) at 25° C. for 10 min. The lipid nanoparticles were then added to the cells and incubated for 6 hours at 37° C. before live cell fluorescence microscopy, shown in FIG. 53. 50 μg/mL Hoechst 33342 was added 30 minutes prior to microscopy. FIG. 53 shows live fluorescence microscopy photos of A549 splitGFP(1-10) cells after incubation with lipid nanoparticles formed by incubating the following conjugates complexed with lipofectamine 2000: Ritux-(pAbBD-S11-DBCO)2, Ritux-(pAbBD-S11-oligo)₂, Ritux-(pAbBD-D25-S11)₂, or Ritux-(pAbBD-E25-S11)₂. Lipo only and Ritux only were used as negative controls. In FIG. 53, the top channel is the Hoechst channel, the middle panel is the splitGFP channel and the bottom panel is the split GFP and Hoechst channel merged.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A composition comprising: an antibody or other protein; an anionic polypeptide; and a cationic transfection agent, wherein said anionic polypeptide comprises a plurality of negatively charged amino acid residues, and wherein the presence of said anionic polypeptide and said cationic transfection agent in said composition facilitate cytoplasmic delivery of said antibody or other protein.

2. The composition of claim 1, wherein said protein is a therapeutic protein.

3. The composition of claim 1, wherein said protein is a protein-based drug or toxin.

4. The composition of claim 1, wherein said protein is an artificial affinity protein.

5. The composition of claim 1, wherein said antibody is a bispecific antibody.

6. The composition of claim 1, wherein said antibody is an immunoglobulin G (IgG) or a fragment thereof.

7. The composition of claim 1, wherein said anionic polypeptide is operably linked to said antibody or other protein.

8. The composition of claim 1, wherein said anionic polypeptide is fused to said antibody or other protein.

9. The composition of claim 1, wherein said antibody is operably linked to an antibody binding domain (AbBD), wherein said AbBD is operably linked or fused to the anionic polypeptide.

10. The composition of claim 9, wherein said antibody binding domain is operably linked to or comprises a photoreactive amino acid group.

11. The composition of claim 10, wherein said photoreactive amino acid is benzoylphenylalanine (BPA).

12. The composition of claim 1, wherein at least 20% of residues in said anionic polypeptide are negatively charged amino acid or unnatural amino acid residues.

13. The composition of claim 1, wherein said negatively charged amino acid residues are aspartic acid residues, glutamic acid residues, or a combination of aspartic acid residues and glutamic acid residues.

14. The composition of claim 1, wherein the number of said plurality of amino acid residues ranges from about 2 to about 50, from about 10 to about 40, from about 20 to about 30, or from about 25 to about 30.

15. The composition of claim 1, wherein said cationic transfection agent is a nano-carrier.

16. The composition of claim 1, wherein said cationic transfection agent is an ionizable carrier.

17. The composition of claim 15, wherein said ionizable carrier includes an ionizable-lipid, polymer, or combination thereof.

18. The composition of claim 15, wherein the ionizable carrier is an ionizable lipid-like nanoparticle.

19. The composition of claim 1, further comprising an agent that induces protein degradation of a target antigen of the antibody.

20. The composition of claim 19, wherein the agent comprises a domain for targeted degradation.

21. The composition of claim 1, further comprising an agent that modifies the function of a target protein.

22. The composition of claim 1, further comprising an agent that induces nuclear, cytoplasmic, membrane or membrane-associated proteins to be sorted into compartments where they are inactive or degraded.

23. A composition comprising: an antibody or other protein; an anionic polypeptide; and an agent that induces a protein degradation, wherein said anionic polypeptide comprises a plurality of negatively charged amino acid residues.

24. The composition of claim 23, further comprising a cationic transfection agent.

25. The composition of claim 23, further comprising an agent that modifies the function of a target protein of the antibody.

26. The composition of claim 23, further comprising an agent that induces nuclear, cytoplasmic, membrane or membrane-associated proteins to be sorted into compartments where they are inactive or degraded.

27. A composition comprising: an antibody or other protein; an anionic nucleic acid; and a cationic transfection agent, wherein the nucleic acid comprises a plurality of negatively charged residues and is an anionic nucleic acid, and wherein presence of the anionic nucleic acid and the cationic transfection agent in the composition facilitate cytoplasmic delivery of the antibody or other protein.

28. The composition of claim 27, wherein the antibody or other protein is operably linked to an antibody binding domain (AbBD), and wherein (i) the antibody or other protein is operably linked to the anionic nucleic acid or (ii) the AbBD is operably linked to the anionic nucleic acid.

29. The composition of claim 27, wherein the protein is a single chain protein.

30. The composition of claim 28, wherein the AbBD is operably linked to or comprises a photoreactive amino acid group.

31. The composition of claim 30, wherein the photoreactive amino acid is benzoylphenylalanine (BPA).

32. The composition of claim 31, wherein the cationic transfection agent is a nano-carrier.

33. The composition of claim 31, wherein the cationic transfection agent is an ionizable carrier.

34. The composition of claim 33, wherein the ionizable carrier includes an ionizable-lipid, polymer, or combination thereof.

35. The composition of claim 33, wherein the ionizable carrier is an ionizable lipid-like nanoparticle.

36. The composition of claim 27, further comprising an agent that induces protein degradation of a target antigen of the antibody.

37. The composition of claim 36, wherein the agent comprises a domain for targeted degradation.

38. The composition of claim 27, further comprising an agent that modifies the function of a target protein.

39. The composition of claim 27, further comprising an agent that induces nuclear, cytoplasmic, membrane or membrane-associated proteins to be sorted into compartments where they are inactive or degraded.

40. The composition of claim 29, wherein the single chain protein is a single chain antibody, a single chain antigen-binding fragment (scFab) or a single chain Fv (scFv).

41. The composition of claim 29, wherein the single chain protein is a single chain targeting ligand.

42. The composition of claim 41, wherein the single chain targeting ligand is an affibody, a nanobody, an antibody mimetic or a peptide.

43. The composition of claim 42, wherein the affibody is an anti-Taq affibody.

44. The composition of claim 42, wherein the nanobody is an anti-GFP nanobody.

45. The composition of claim 42, wherein the antibody mimetic is a genetically engineered designed ankyrin repeat protein (DARPin).

46. The composition of claim 42, wherein the peptide comprises Omomycin.

47. A method of delivering an antibody or other protein to cytoplasm of a cell in a subject, comprising:
providing the composition according to any one of claims 1-46; and
administering said composition to said subject.

48. A method of treating a disease or disorder in a subject, comprising: delivering a therapeutic composition in a cytoplasm of a cell of said subject, wherein said therapeutic composition comprises the composition according to any one of claims 1-46.

49. A method of manufacturing a composition for cytoplasmic delivery, comprising:
(i) covalently linking, ligating, or fusing an antibody or a protein molecule to an anionic polypeptide in order to prepare a conjugate or
(ii) covalently linking, ligating, or fusing a protein to an anionic nucleic acid; and
(iii) mixing or complexing a cationic transfection agent with the conjugate of (i) or (ii).

50. A cell recombinantly expressing the composition according to any one of claims 1-46.

51. A method of manufacturing a composition for cytoplasmic delivery, comprising:
providing the cell of claim 50; and
expressing the composition.

52. A conjugate comprising:
(i) an antibody binding domain (AbBD) operably linked, ligated or fused to an anionic polypeptide comprising a plurality of negatively charged amino acid residues; or
(ii) a protein operably linked, ligated or fused to an anionic nucleic acid.

53. The conjugate of claim 52, wherein the AbBD is operably linked to the anionic nucleic acid.

54. The conjugate of claim 52, wherein the AbBD is operably linked to or comprises a photoreactive amino acid group.

55. The conjugate of claim 52, wherein the protein operably linked to the anionic nucleic acid is operably linked to or comprises a photoreactive amino acid group.

56. The conjugate according to any one of claims 54-55, wherein the photoreactive amino acid is benzoylphenylalanine.

57. The conjugate of claim 52, wherein at least 20% of residues in the anionic polypeptide are negatively charged amino acid or unnatural amino acid residues.

58. The conjugate of claim 52, wherein the protein operably linked to the anionic nucleic acid is a single chain protein.

59. The conjugate of claim 52, wherein the protein operably linked to the anionic nucleic acid is an antibody.

60. The conjugate of claim 52, wherein the negatively charged amino acid residues are aspartic acid residues, glutamic acid residues, or a combination of aspartic acid residues and glutamic acid residues.

61. The conjugate of claim 52, wherein the number of the plurality of amino acid residues ranges from about 2 to about 50, from about 10 to about 40, from about 20 to about 30, or from about 25 to about 30.

62. The conjugate of claim 52, further comprising a cationic transfection agent or further mixed/complexed with the cationic transfection agent.

63. The conjugate of claim 52, further comprising an agent that modifies the function of a target protein.

64. The conjugate of claim 52, further comprising an agent that induces nuclear, cytoplasmic, membrane or membrane-associated proteins to be sorted into compartments where they are inactive or degraded.

65. The conjugate of claim 52, further comprising an agent that induces a protein degradation of a target antigen of the antibody.

66. The conjugate of claim 65, wherein the agent comprises a domain for targeted degradation.

67. A cell recombinantly expressing the conjugate according to any one of claims 52-66.

68. A method for sensitizing a tumor cell to a chemotherapeutic agent, the method comprising administering to cytoplasm of the tumor cell:
(i) a conjugate according to any one of claims 52-66; or
(ii) a cell recombinantly expressing the conjugate of (i).

69. The method of claim 68, wherein the AbBD comprises an AbBD of anti-human multidrug resistance-associated protein 1 (MRP1) monoclonal antibody QCRL3, wherein the AbBD of anti-human MRP1 binds to conformation-dependent internal epitope of human MRP1, the epitope comprising amino acids 617-932 of human MRP1.

70. The method of claim 68, wherein the chemotherapeutic agent is doxorubicin or vincristine.

71. The method of claim 68, wherein the tumor cell is a multidrug resistant (MDR) tumor cell.

72. The method of claim 71, wherein the MDR tumor cell is a human non-P-glycoprotein MDR tumor cell.

73. A method for decreasing or inhibiting growth of a tumor cell, the method comprising administering to cytoplasm of the tumor cell in a subject in need thereof the composition according to any one of claims 1-46.

74. The method of claim 73, wherein the other protein comprises a genetically engineered designed ankyrin repeat protein (DARPin).

75. The method of claim 73, wherein the other protein comprises Omomycin.

76. The method of claim 73, wherein the AbBD comprises an AbBD of anti-human multidrug resistance-associated protein 1 (MRP1) monoclonal antibody QCRL3.

77. The method of claim 73, wherein the tumor cell is a multidrug resistant (MDR) tumor cell.

78. The method of claim 77, wherein the MDR tumor cell is a human non-P-glycoprotein MDR tumor cell.

79. A method for inhibiting NF-kB transcription and/or reducing RelA nuclear translocation a cancer cell, the method comprising administering to cytoplasm of the cancer cell in a subject in need thereof the composition according to any one of claims 1-46.

80. The method of claim 79, wherein the AbBD comprises an AbBD of anti-RelA antibody, wherein the antibody is an IgG.
